(19) United States
(12) Patent Application Publication
(10) Pub. No.: US 2007/0093529 A1
(43) Pub. Date: Apr. 26, 2007

(54) Diacylhydrazine derivatives

(76) Inventors: Dirk Finsinger, Darmstadt (DE); Hans-Peter Buchstaller, Weiterstadt (DE); Lars Burgdorf, Frankfurt/Main (DE); Christiane Amendt, Darmstadt (DE); Matthias Grell, Darmstadt (DE); Christian Sirrenberg, Darmstadt (DE); Frank Zenke, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/582,496

(22) PCT Filed: Nov. 11, 2004
(86) PCT No.: PCT/EP04/12764

§ 371 Date: Jun. 9, 2006

(30) Foreign Application Priority Data
Dec. 10, 2003 (EP) 03028268.5

(51) Int. Cl. C07D 403/02; A61K 31/4439; A61K 31/4412

(52) U.S. Cl. 514/337; 514/357; 546/332; 514/346; 546/277.1; 546/294

(57) ABSTRACT

The present invention relates to diacylhydrazine derivatives of formula I, the use of the compounds of formula I as inhibitors of raf-kinase, the use of the compounds of formula I for the manufacture of a pharmaceutical composition and a method of treatment, comprising administering said pharmaceutical composition to a patient.

The present invention relates to diacylhydrazine derivatives, diacylhydrazine derivatives as medicaments, diacylhydrazine derivatives as inhibitors of raf-kinase, the use of diacylhydrazine derivatives for the manufacture of a pharmaceutical, a method for producing a pharmaceutical composition containing said diacylhydrazine derivatives, the pharmaceutical composition obtainable by said method and a method of treatment, comprising administering said pharmaceutical composition.

Protein phosphorylation is a fundamental process for the regulation of cellular functions. The coordinated action of both protein kinases and phosphatases controls the levels of phosphorylation and, hence, the activity of specific target proteins. One of the predominant roles of protein phosphorylation is in signal transduction, where extracellular signals are amplified and propagated by a cascade of protein phosphorylation and dephosphorylation events, e.g. in the p21^{ras}/raf pathway.

The p21^{ras} gene was discovered as an oncogene of the Harvey (rasH) and Kirsten (rasK) rat sarcoma viruses. In humans, characteristic mutations in the cellular ras gene (c-ras) have been associated with many different types of cancers. These mutant alleles, which render Ras constitutively active, have been shown to transform cells, such as the murine cell line NIH 3T3, in culture.

The p21^{ras} oncogene is a major contributor to the development and progression of human solid cancers and is mutated in 30% of all human cancers (Bolton et al. (1994) Ann. Rep. Med. Chem., 29, 165-74; Bos. (1989) Cancer Res., 49, 4682-9). Oncogenic Ras mutations have been identified for example in lung cancer, colorectal cancer, pancreas, thyroid cancer, melanoma, bladder tumors, liver tumor, kidney tumor, dermatological tumors and haematological tumors (Ddjei et al. (2001), J. Natl. Cancer Inst. 93(14), 1062-74; Midgley, R. S. and Kerr, D. J. (2002) Critical Rev. Onc/hematol 44, 109-120; Downward, J. (2003), Nature reviews 3, 11-22). In its normal, unmutated form, the ras protein is a key element of the signal transduction cascade directed by growth factor receptors in almost all tissues (Avruch et al. (1994) Trends Biochem. Sci., 19, 279-83).

Biochemically, ras is a guanine nucleotide binding protein, and cycling between a GTP-bound activated and a GDP-bound resting form is strictly controlled by ras endogenous GTPase activity and other regulatory proteins. The ras gene product binds to guanine triphosphate (GTP) and guanine diphosphate (GDP) and hydrolyzes GTP to GDP. It is the GTP-bound state of Ras that is active. In the ras mutants in cancer cells, the endogenous GTPase activity is alleviated and, therefore, the protein delivers constitutive growth signals to downstream effectors such as the enzyme raf kinase. This leads to the cancerous growth of the cells which carry these mutants (Magnuson et al. (1994) Semin. Cancer Biol., 5, 247-53). The ras proto-oncogene requires a functionally intact c-raf1 proto-oncogene in order to transduce growth and differentiation signals initiated by receptor and non-receptor tyrosine kinases in higher eukaryotes.

Activated Ras is necessary for the activation of the c-raf-1 proto-oncogene, but the biochemical steps through which Ras activates the Raf-1 protein (Ser/Thr) kinase are now well characterized. It has been shown that inhibiting the effect of active ras by inhibiting the raf kinase signaling pathway by administration of deactivating antibodies to raf kinase or by co-expression of dominant negative raf kinase or dominant negative MEK also called ERK, the substrate of raf kinase, leads to the reversion of transformed cells to the normal growth phenotype see: Daum et al. (1994) Trends Biochem. Sci., 19, 474-80; Fridman et al. (1994) J. Biol. Chem., 269, 30105-8. Kolch et al. (1991) Nature, 349, 426-28) and for review Weinstein-Oppenheimer et al. Pharm. & Therap. (2000), 88, 229-279. Similarly, inhibition of raf kinase (by antisense oligodeoxynucleotides) has been correlated in vitro and in vivo with inhibition of the growth of a variety of human tumor types (Monia et al., Nat. Med. 1996, 2, 668-75; Geiger et al. (1997), Clin. Cancer Res. 3(7): 1179-85; Lau et al. (2002), Antisense Nucl. Acid. Drug Dev. 12(1): 11-20; McPhillips et al. (2001), Br. J. Cancer 85(11): 1753-8).

Raf serine- and threonine-specific protein kinases are cytosolic enzymes that stimulate cell growth in a variety of cell systems (Rapp, U. R., et al. (1988) in The oncogene handbook; T. Curran, E. P. Reddy, and A. Skalka (ed.) Elsevier Science Publishers; The Netherlands, pp. 213-253; Rapp, U. R., et al. (1988) Cold Spring Harbor Sym. Quant. Biol. 53:173-184; Rapp, U. R., et al. (1990) Inv Curr. Top. Microbiol. Amunol. Potter and Melchers (eds), Berlin, Springer-Verlag 166:129-139).

Three isozymes have been characterized:

c-Raf (also named Raf-1, c-raf-1 or c-raf1) (Bonner, T. I., et al. (1986) Nucleic Acids Res. 14:1009-1015). A-Raf (Beck, T. W., et al. (1987) Nucleic Acids Res. 15:595-609), and B-Raf (Qkawa, S., et al. (1998) Mol. Cell. Biol. 8:2651-2654; Sithanandam, G. et al. (1990) Oncogene:1775). These enzymes differ in their expression in various tissues. Raf-1 is expressed in all organs and in all cell lines that have been examined, and A- and B-Raf are expressed in urogenital and brain tissues, respectively (Storm, S. M. (1990) Oncogene 5:345-351).

Raf genes are proto-oncogenes: they can initiate malignant transformation of cells when expressed in specifically altered forms. Genetic changes that lead to oncogenic activation generate a constitutively active protein kinase by removal or interference with an N-terminal negative regulatory domain of the protein (Heidecker, G., et al. (1990) Mol. Cell. Biol. 10:2503-2512; Rapp, U. R., et al. (1987) in Oncogenes and cancer S. A. Aaronson, J. Bishop, T. Sugimura, M. Terada, K. Toyoshima, and P. K. Vogt (ed). Japan Scientific Press, Tokyo). Microinjection into NIH 3T3 cells of oncogenically activated but not wild-type versions of the Raf-protein prepared with Escherichia coli expression vectors results in morphological transformation and stimulates DNA synthesis (Rapp, U. R., et al. (1987) in Oncogenes and cancer; S. A. Aaronson, J. Bishop, T. Sugimura, M. Terada, K. Toyoshima, and P. K. Vogt (ed.) Japan Scientific Press, Tokyo; Smith, M. R., et al (1990) Mol. Cell. Biol. 10:3828-3833). Activating mutants of B-Raf have been identified in a wide range of human cancers e.g. colon, ovarian, melanomas and sarcomas (Davies, H., et al. (2002), Nature 417 949-945. Published online Jun. 9, 2002, 10.1038/nature00766). The preponderant mutation is a single phosphomimetic substitution in the kinase activation domain (V599E), leading to constitutive kinase activity and transformation of NIH3T3 cells.

Thus, activated Raf-1 is an intracellular activator of cell growth. Raf-1 protein serine kinase in a candidate downstream effector of mitogen signal transduction, since Raf oncogenes overcome growth arrest resulting from a block of cellular ras activity due either to a cellular mutation (ras revertant cells) or microinjection of anti-ras antibodies (Rapp, U. R., et al. (1988) in The Oncogene Handbook, T. Curran, E. P. Reddy, and A. Skalka (ed.), Elsevier Science Publishers; The Netherlands, pp. 213-253; Smith, M. R., et al. (1986) Nature (London) 320:540-543).

c-Raf function is required for transformation by a variety of membrane-bound oncogenes and for growth stimulation by mitogens contained in serums (Smith, M. R., et al. (1986) Nature (London) 320:540-543). Raf-1 protein serine kinase activity is regulated by mitogens via phosphorylation (Morrison, D. K., et al. (1989) Cell 58:648-657), which also effects sub cellular distribution (Olah, Z., et al. (1991) Exp. Brain Res. 84:403; Rapp, U. R., et al. (1988) Cold Spring Harbor Sym. Quant. Biol. 53:173-184. Raf-1 activating growth factors include platelet-derived growth factor (PDGF) (Morrison, D. K., et al. (1988) Proc. Natl. Acad. Sci. USA 85:8855-8859), colony-stimulating factor (Baccarini, M., et al. (1990) EMBO J. 9:3649-3657), insulin (Blackshear, P. J., et al. (1990) J. Biol. Chem. 265:12115-12118), epidermal growth factor (EGF) (Morrison, R. K., et al. (1988) Proc. Natl. Acad. Sci. USA 85:8855-8859), interleukin 2 (Turner, B. C., et al (1991) Proc. Natl. Acad. Sci. USA 88:1227), and interleukin 3 and granulocytemacrophage colony-stimulating factor (Carroll, M. P., et al (1990) J. Biol. Chem. 265:19812-19817).

Upon mitogen treatment of cells, the transiently activated Raf-1 protein serine kinase translocates to the perinuclear area and the nucleus (Olah, Z., et al. (1991) Exp. Brain Res. 84:403; Rapp, U. R., et al. (1988) Cold Spring Habor Sym. Quant. Biol. 53:173-184). Cells containing activated Raf are altered in their pattern of gene expression (Heidecker, G., et al. (1989) in Genes and signal transduction in multistage carcinogenesis, N. Colburn (ed.), Marcel Dekker, Inc., New York, pp. 339-374), and Raf oncogenes activate transcription from Ap-I/PEA3-dependent promoters in transient transfection assays (Jamal, S., et al (1990) Science 344:463-466; Kaibuchi, K., et al (1989) J. Biol. Chem. 264:20855-20858; Wasylyk, C., et al. (1989) Mol. Cell. Biol. 9:2247-2250).

There are at least two independent pathways for Raf-1 activation by extracellular mitogens: one involving protein kinase C (KC) and a second initiated by protein tyrosine kinases (Blackshear, P. J., et al. (1990) J. Biol. Chem. 265:12131-12134; Kovacina, K. S., et al (1990) J. Biol. Chem. 265:12115-12118; Morrison, D. K., et al. (1988) Proc. Natl. Acad. Sci. USA 85:8855-8859; Siegel, J. N., et al (1990) J. Biol. Chem. 265:18472-18480; Turner, B. C., et al (1991) Proc. Natl. Acad. Sci. USA 88:1227). In either case, activation involves Raf-1 protein phosphorylation. Raf-1 phosphorylation may be a consequence of a kinase cascade amplified by autophosphorylation or may be caused entirely by autophosphorylation initiated by binding of a putative activating ligand to the Raf-1 regulatory domain, analogous to PKC activation by diacylglycerol (Nishizuka, Y. (1986) Science 233:305-312).

The process of angiogenesis is the development of new blood vessels, generally capillaries, from pre-existing vasculature. Angiogenesis is defined as involving (i) activation of endothelial cells; (ii) increased vascular permeability; (iii) subsequent dissolution of the basement membrane and extravisation of plasma components leading to formation of a provisional fibrin gel extracellular matrix; (iv) proliferation and mobilization of endothelial cells; (v) reorganization of mobilized endothelial cells to form functional capillaries; (vi) capillary loop formation; and (vii) deposition of basement membrane and recruitment of perivascular cells to newly formed vessels.

Normal angiogenesis is activated during tissue growth, from embryonic development through maturity, and then enters a period of relative quiescence during adulthood.

Normal angiogenesis is also activated during wound healing, and at certain stages of the female reproductive cycle. Inappropriate or pathological angiogenesis has been associated with several disease states including various retinopathies; ischemic disease; atherosclerosis; chronic inflammatory disorders; rheumatoid arthritis, and cancer. The role of angiogenesis in disease states is discussed, for instance, in Fan et al, Trends in Pharmacol Sci. 16:54 66; Shawver et al, DOT Vol. 2, No. 2 Feb. 1997; Folkmann, 1995, Nature Medicine 1:27-31.

In cancer the growth of solid tumors has been shown to be angiogenesis dependent. (See Folkmann, J., J. Nat'l. Cancer Inst., 1990, 82, 4-6.) Consequently, the targeting of pro-angiogenic pathways is a strategy being widely pursued in order to provide new therapeutics in these areas of great, unmet medical need.

Raf is involved in angiogenic processes. Endothelial growth factors (e.g. vascular endothelial growth factor VEGF or basic fibroblast growth factor bFGF) activates receptor tyrosine kinases (e.g. VEGFR-2) and signal through the Ras/Raf/Mek/Erk kinase cascade and protects endothelial cells from apoptosis (Alavi et al. (2003), Science 301, 94-96; Hood, J. D. et al. (2002), Science 296; 2404; Mikula, M. et al. (2001), EMBO J. 20, 1952; Hauser, M. et al. (2001), EMBO J. 20, 1940; Wojnowski et al. (1997), Nature Genet. 16, 293). Activation of VEGFR-2 by VEGF is a critical step in the signal transduction pathway that initiates tumor angiogenesis. VEGF expression may be constitutive to tumor cells and can also be upregulated in response to certain stimuli. One such stimuli is hypoxia, where VEGF expression is upregulated in both tumor and associated host tissues. The VEGF ligand activates VEGFR-2 by binding with its extracellular VEGF binding site. This leads to receptor dimerization of VEGFRs and autophosphorylation of tyrosine residues at the intracellular kinase domain of VEGFR-2. The kinase domain operates to transfer a phosphate from ATP to the tyrosine residues, thus providing binding sites for signaling proteins downstream of VEGFR-2 leading ultimately to initiation of angiogenesis (McMahon, G., The Oncologist, Vol. 5, No. 90001, 3-10, Apr. 2000).

Mice with a targeted disruption in the Braf gene die of vascular defects during development (Wojnowski, L. et al. 1997, Nature genetics 16, page 293-296). These mice show defects in the formation of the vascular system and in angiogenesis e.g. enlarged blood vessels and increased apoptotic death of differentiated endothelial cells.

For the identification of a signal transduction pathway and the detection of cross talks with other signaling pathways suitable models or model systems have been generated by various scientists, for example cell culture models (e.g. Khwaja et al., EMBO, 1997, 16, 2783-93) and transgenic animal models (e.g. White et al., Oncogene, 2001, 20, 7064-7072). For the examination of particular steps in the signal transduction cascade, interfering compounds can be used for signal modulation (e.g. Stephens et al., Biochemical J., 2000, 351, 95-105). The compounds according to the invention may also be useful as reagents for the examination of kinase dependent signal transduction pathways in animal and/or cell culture models or any of the clinical disorders listed throughout this application.

The measurement of kinase activity is a well known technique feasible for each person skilled in the art. Generic test systems for kinase activity detection with substrates, for example histone (e.g. Alessi et al., FEBS Lett. 1996, 399, 3, page 333-8) or myelin basic protein are well described in the literature (e.g. Campos-Gonzalez, R. and Glenney, Jr., J. R. 1992 J. Biol. Chem. 267, Page 14535).

For the identification of kinase inhibitors various assay systems are available (see for example Walters et al., Nature Drug Discovery 2003, 2; page 259-266). For example, in scintillation proximity assays (e.g. Sorg et al., J. of. Biomolecular Screening, 2002, 7, 11-19) or flashplate assays the radioactive phosphorylation of a protein or peptide as substrate with □ATP can be measured. In the presence of an inhibitory compound no signal or a decreased radioactive signal is detectable. Furthermore homogeneous time-resolved fluorescence resonance energy transfer (HTR-FRET), and fluorescence polarization (FP) technologies are useful for assay methods (for example Sills et al., J. of Biomolecular Screening, 2002, 191-214).

Other non-radioactive ELISA based assay methods use specific phospho-antibodies (AB). The phospho-AB binds only the phosphorylated substrate.

This binding is detectable with a secondary peroxidase conjugated antibody, measured for example by chemiluminescence (for example Ross et al., Biochem. J., 2002, 366, 977-981).

The present invention provides compounds generally described as diacylhydrazine derivatives, including both aryl and/or heteroaryl derivatives which are preferably kinase inhibitors and more preferably inhibitors of the enzyme raf kinase. Since the enzyme is a downstream effector of p21^{ras}, the inhibitors are useful in pharmaceutical compositions for human or veterinary use where inhibition of the raf kinase pathway is indicated, e.g., in the treatment of tumors and/or cancerous cell growth mediated by raf kinase. In particular, the compounds are useful in the treatment of human or animal solid cancers, e.g. murine cancer, since the progression of these cancers is dependent upon the ras protein signal transduction cascade and therefore susceptible to treatment by interruption of the cascade, i.e., by inhibiting raf kinase. Accordingly, the compound of Formula I or a pharmaceutically acceptable salt thereof is administered for the treatment of diseases mediated by the raf kinase pathway especially cancers, including solid cancers, such as, for example, carcinomas (e.g., of the lungs, pancreas, thyroid, bladder or colon), myeloid disorders (e.g., myeloid leukemia) or adenomas (e.g., villous colon adenoma), pathological angiogenesis and metastatic cell migration. Furthermore the compounds are useful in the treatment of complement activation dependent chronic inflammation (Niculescu et al. (2002) Immunol. Res., 24:191-199) and HIV-1 (human immunodeficiency virus type1) induced immunodeficiency (Popik et al. (1998) J Virol, 72: 6406-6413) and infection disease, Influenza A virus (Pleschka, S. et al. (2001), Nat. Cell. Biol, 3(3):301-5) and Helicobacter pylori infection (Wessler, S. et al. (2002), FASEB J., 16(3): 417-9).

Therefore, subject of the present invention are diacylhydrazine derivatives of formula I
A-D-B (I)
wherein
D is a bivalent diacylhydrazine moiety which is directly bonded to A and B, wherein the carbonyl group of said diacylhydrazine moiety can be derivatized, preferably to a C═S, C═NR⁵, C═C(R⁵)—NO₂, C═C(R⁵)—CN or C═C(CN)₂ group
A is a unsubstituted or preferably substituted moiety of up to 40 carbon atoms of the formula: -L-(M-L′)_{α}, where L is a 5, 6 or 7 membered cyclic structure, preferably selected from the group consisting of aryl, heteroaryl, arylene and heteroarylene, bound directly to D, L′ comprises an optionally substituted cyclic moiety having at least 5 members, preferably selected from the group consisting of aryl, heteroaryl, aralkyl, cycloalkyl and heterocyclyl, M is a bond or a bridging group having at least to one atom, α is an integer of from 1-4; and each cyclic structure of L and L′ contains 04 members of the group consisting of nitrogen, oxygen and sulfur, wherein L′ is preferably substituted by at least one substituent selected from the group consisting of —SO_{β}Rₓ, —C(O)Rₓ and —C(NR_{y})R_{z},
B is a substituted or unsubstituted, up to tricyclic aryl or heteroaryl moiety of up to 30 carbon atoms, preferably of up to 20 carbon atoms, comprising at least one 5-, 6-, or 7-membered cyclic structure, preferably a 5 or 6-membered cyclic structure, bound directly to D containing 0-4 members of the group consisting of nitrogen, oxygen and sulfur, wherein said cyclic structure directly bound to D is preferably selected from the group consisting of aryl, heteroaryl and heterocyclyl,
R_{y} is hydrogen or a carbon based moiety of up to 24 carbon atoms optionally containing heteroatoms selected from N, S and O and optionally halosubstituted, up to per halo,
R_{z} is hydrogen or a carbon based moiety of up to 30 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen;
Rₓ is R_{z} or NRₐR_{b}, where Rₐ and R_{b} are
   a) independently hydrogen, a carbon based moiety of up to 30 carbon atoms optionally containing heteroatoms selected from N, S and O and optionally substituted by halogen, hydroxy and carbon based substituents of up to 24 carbon atoms, which optionally contain heteroatoms, selected from N, S and O, and are optionally substituted by halogen, or
      —OSi(R_{f})₃ where R_{f} is hydrogen or a carbon based moiety of up to 24 carbon atoms optionally containing heteroatoms selected from N, S and O and optionally substituted by halogen, hydroxy and carbon based substituents of up to 24 carbon atoms, which optionally contain heteroatoms selected from N, S and O, and are optionally substituted by halogen; or
   b) Rₐ and R_{b} together from a 5-7 member heterocyclic structure of 1-3 heteroatoms selected from N, S and O, or a substituted 5-7 member heterocyclic structure of 1-3 heteroatoms selected from N, S and O substituted by halogen, hydroxy or carbon based substituents of up to 24 carbon atoms, which optionally contain heteroatoms selected from N, S and O and are optionally substituted by halogen; or
   c) one of Rₐ or R_{b} is —C(O)—, a C₁-C₅ divalent alkylene group or a substituted C₁-C₅ divalent alkylene group bound to the moiety L to form a cyclic structure with at least 5 members, wherein the substituents of the substituted C₁-C₅ divalent alkylene group are selected from the group consisting of halogen, hydroxy, and carbon based substituents of up to 24 carbon atoms, which optionally contain heteroatoms selected from N, S and O and are optionally substituted by halogen; where B is substituted, L is substituted or L′ is additionally substituted, the substituents are selected from the group consisting of halogen, up to per-halo and Wγ, where γ is 0-3;
      wherein each W is independently selected from the group consisting of —CN, —CO₂R, —C(O)NR⁵R⁵, —C(O)—R⁵, —NO₂, —OR⁵, —SR⁵, —NR⁵R⁵, —NR⁵C(O)OR⁵, —NR⁵C(O)R⁵, -Q-Ar, and carbon based moieties of up to 24 carbon atoms, optionally containing heteroatoms selected from N, S and O and optionally substituted by one or more substituents independently selected from the group consisting of —CN, —CO₂R, —C(O)NR⁵R⁵, —C(O)—R⁵, —NO₂, —OR⁵, —SR⁵, —NR⁵R⁵, —NR⁵C(O)OR⁵, —NR⁵C(O)R⁵ and halogen up to per-halo; with each R⁵ independently selected from H or a carbon based moiety of up to 24 carbon atoms, optionally containing heteroatoms selected from N, S
   d) and O and optionally substituted by halogen;
      wherein Q is —O—, —S—, —N(R⁵)—, —(CH₂)_{β}, —C(O)—, —CH(OH)—, —(CH₂)_{β}—, —(CH₂)_{β}S—, —(CH₂)_{β}N(R⁵)—, —O(CH₂)_{β}—CHHal-, —CHal₂-, —S—(CH₂)— and —N(R⁵)(CH₂)_{β}— where β=1-3, and Hal is halogen; and
      Ar is a 5- or 6-member aromatic structure containing 0-2 members selected from the group consisting of nitrogen, oxygen and sulfur, which is optionally substituted by halogen, up to per-halo, and optionally substituted by 41 wherein 61 is 0 to 3 and each Z is independently selected from the group consisting —CN, —CO₂R⁵, —C(O)NR⁵R⁵, —C(O)—R⁵, —NO₂, —OR⁵, —SR⁵—NR⁵R⁵, —NR⁵C(O)OR⁵, —NR⁵C(O)R⁵, and a carbon based moiety of up to 24 carbon atoms, optionally containing heteroatoms selected from N, A and O and optionally substituted by one or more substituents selected from the group consisting of —CN, —CO₂R⁵, —C(O)NR⁵R⁵, —C(O)—R⁵, —NO₂, —OR⁵, —SR⁵, —NR⁵R⁵, —NR⁵C(O)OR⁵, —NR⁵C(O)R⁵, and with R⁵ as defined above,
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

More preferred, in the compound of formula I
R_{y} is hydrogen, C₁₋₁₀ alkyl, C₁₋₁₀ alkoxy, C₃₋₁₀ cycloalkyl having 0-3 heteroatoms, C₂₋₁₀ alkenyl, C₁₋₁₀ alkenoyl, C₆₋₁₂ aryl, C₃₋₁₂ hetaryl having 1-3 heteroatoms selected from N, S and O, C₇₋₂₄ aralkyl, C₇₋₂₄ alkaryl, substituted C₁₋₁₀ alkyl, substituted C₁₋₁₀ alkoxy, substituted C₃₋₁₀ cycloalkyl having 0-3 heteroatoms selected from N, S and O, substituted C₆-C₁₄ aryl, substituted C₃₋₁₂ hetaryl having 1-3 heteroatoms selected from N, S and O, substituted C₇₋₂₄ alkaryl or substituted C₇₋₂₄ aralkyl, where R_{y} is a substituted group, it is substituted by halogen up to per halo,
R_{z} is hydrogen, C₁₋₁₀ alkyl, C₁₋₁₀ alkoxy, C₃₋₁₀ cycloalkyl having 0-3 heteroatoms, C₂₋₁₀ alkenyl, C₁₋₁₀ alkenoyl, C₆₋₁₂ aryl, C₃-C₁₂ hetaryl having 1-3 heteroatoms selected form S, N and O, C₇₋₂₄ alkaryl, C₇₋₂₄ aralkyl, substituted C₃-C₁₀ cycloalkyl having 0-3 heteroatoms selected from S, N and O, substituted C₃₋₁₂ hetaryl having 1-3 heteroatoms selected from S, N and O, substituted C₇₋₂₄ alkaryl or substituted C₇-C₂₄ aralkyl, where R_{z} is a substituted group, it is substituted by halogen up to per halo, hydroxy, C₁₋₁₀ alkyl, C₃₋₁₂ cycloalkyl having 0-3 heteroatoms selected from N, S and O, substituted C₃-C₁₂ hetaryl having 1-3 heteroatoms selected from N, S and O, C₁₋₁₀ alkoxy, C₇₋₁₂ aryl, C₁₋₆ halo substituted alkyl up to per halo alkyl, C₆-C₁₂ halo substituted aryl up to per halo aryl, C₃-C₁₂ halo substituted cycloalkyl up to per halo cycloalkyl having 0-3 heteroatoms selected from N, S and O, halo substituted C₃-C₁₂ hetaryl up to per halo, hetaryl having 1-3 heteroatoms selected from O, N and S, halo substituted C₇-C₂₄ aralkyl up to per halo aralkyl, halo substituted C₇-C₂₄ alkaryl up to per halo alkaryl, and —C(O)R_{g},
Rₐ and R_{b} are,
   a) independently hydrogen, a carbon based moiety selected from the group consisting of C₁-C₁₀ alkyl, C₁-C₁₀ alkoxy, C₃₋₁₀ cycloalkyl, C₂₋₁₀ alkenyl, C₁₋₁₀ alkenoyl, C₆₋₁₂ aryl, C₃₋₁₂ hetaryl having 1-3 heteroatoms selected from O, N and S, C₃₋₁₂ cycloalkyl having 0-3 heteroatoms selected from N, S and O, C₇₋₂₄ aralkyl, C₇-C₂₄ alkaryl, substituted C₁₋₁₀ alkyl, substituted C₁₋₁₀ alkoxy, substituted C₃₋₁₀ cycloalkyl, having 0-3 heteroatoms selected from N, S and O, substituted C₆₋₁₂ aryl, substituted C₃₋₁₂ hetaryl having 1-3 heteroatoms selected from N, S and O, substituted C₇₋₂₄ aralkyl, substituted C₇₋₂₄ alkaryl, where Rₐ and R_{b} are a substituted group, they are substituted by halogen up to per halo, hydroxy, C₁₋₁₀ alkyl, C₃₋₁₂ cycloalkyl having 0-3 heteroatoms selected from O, S and N, C₃₋₁₂ hetaryl having 1-3 heteroatoms selected from N, S and O, C₁₋₁₀ alkoxy, C₆₋₁₂ aryl, C₁₋₆ halo substituted alkyl up to per halo alkyl, C₆-C₁₂ halo substituted aryl up to per halo aryl, C₃-C₁₂ halo substituted cycloalkyl having 0-3 heteroatoms selected from N, S and O, up to per halo cycloalkyl, halo substituted C₃-C₁₂ hetaryl up to per halo heteraryl, halo substituted C₇-C₂₄ aralkyl up to per halo aralkyl, halo substituted C₇-C₂₄ alkaryl up to per halo alkaryl, and —C(O)R_{g}; or
      —OSi(R_{f})₃ where R_{f} is hydrogen, C₁-C₁₀ alkyl, C₁-C₁₀ alkoxy, C₃₋₁₀ cycloalkyl, C₂₋₁₀ alkenyl, C₁₋₁₀ alkenoyl, C₆₋₁₂ aryl, C₃₋₁₂ hetaryl having 1-3 heteroatoms selected from O, N and S, C₃₋₁₂ cycloalkyl having 0-3 heteroatoms selected from N, S and O, C₇₋₂₄ aralkyl, C₇-C₂₄ alkaryl, substituted C₁₋₁₀ alkyl, substituted C₁₋₁₀ alkoxy, substituted C₃₋₁₀ cycloalkyl, having 0-3 heteroatoms selected from N, S and O, substituted C₆₋₁₂ aryl, substituted C₃₋₁₂ hetaryl having 1-3 heteroatoms selected from N, S and O, substituted C₇₋₂₄ aralkyl, substituted C₇₋₂₄ alkaryl, where Rₐ and R_{b} are a substituted group, they are substituted by halogen up to per halo, hydroxy, C₁₋₁₀ alkyl, C₃₋₁₂ cycloalkyl having 0-3 heteroatoms selected from O, S and N, C₃₋₁₂ hetaryl having 1-3 heteroatoms selected from N, S and O, C₁₋₁₀ alkoxy, C₆₋₁₂ aryl, C₁₋₄ halo substituted alkyl up to per halo alkyl, C₆-C₁₂ halo substituted aryl up to per halo aryl, C₃-C₁₂ halo substituted cycloalkyl having 0-3 heteroatoms selected from N, S and O, up to per halo cycloalkyl, halo substituted C₃-C₁₂ hetaryl up to per halo heteraryl, halo substituted C₇-C₂₄ aralkyl up to per halo aralkyl, halo substituted C₇-C₂₄ alkaryl up to per halo alkaryl, and —C(O)R_{g},
      or
   b) Rₐ and R_{b} together form a 5-7 member heterocyclic structure of 1-3 heteroatoms selected from N, S and O, or a substituted 5-7 member heterocyclic structure of 1-3 heteroatoms selected from N, S and O with substituents selected from the group consisting of halogen up to per halo, hydroxy, C₁-C₁₀ alkyl, C₁-C₁₀ alkoxy, C₃₋₁₀ cycloalkyl, C₂₋₁₀ alkenyl, C₁₋₁₀ alkenoyl, C₆₋₁₂ aryl, C₃₋₁₂ hetaryl having 1-3 heteroatoms selected from O, N and S, C₃₋₁₂ cycloalkyl having 0-3 heteroatoms selected from N, S and O, C₇₋₂₄ aralkyl, C₇-C₂₄ alkaryl, substituted C₁₋₁₀ alkyl, substituted C₁₋₁₀ alkoxy, substituted C₃₋₁₀ cycloalkyl, having 0-3 heteroatoms selected from N, S and O, substituted C₆₋₁₂ aryl, substituted C₃₋₁₂ hetaryl having 1-3 heteroatoms selected from N, S and O, substituted C₇₋₂₄ aralkyl, substituted C₇₋₂₄ alkaryl, where Rₐ and R_{b} are a substituted group, they are substituted by halogen up
   c) to per halo, hydroxy, C₁₋₁₀ alkyl, C₃₋₁₂ cycloalkyl having 0-3 heteroatoms selected from O, S and N, C₃₋₁₂ hetaryl having 1-3 heteroatoms selected from N, S and O, C₁₋₁₀ alkoxy, C₆₋₁₂ aryl, C₁₋₆ halo substituted alkyl up to per halo alkyl, C₆-C₁₂ halo substituted aryl up to per halo aryl, C₃-C₁₂ halo substituted cycloalkyl having 0-3 heteroatoms selected from N, S and O, up to per halo cycloalkyl, halo substituted C₃-C₁₂ hetaryl up to per halo heteraryl, halo substituted C₇-C₂₄ aralkyl up to per halo aralkyl, halo substituted C₇-C₂₄ alkaryl up to per halo alkaryl, and —C(O)R_{g},
      or
   d) one of Rₐ or R_{b} is —C(O)—, a C₁-C₅ divalent alkylene group or a substituted C₁-C₅ divalent alkylene group bound to the moiety L to form a cyclic structure with at least 5 members, wherein the substituents of the substituted C₁-C₅ divalent alkylene group are selected from the group consisting of halogen, hydroxy, C₁₋₁₀ alkyl, C₃₋₁₂ cycloalkyl having 0-3 heteroatoms selected from, S and N, C₃₋₁₂ hetaryl having 1-3 heteroatoms selected from N, S and O, C₁₋₁₀ alkoxy, C₆₋₁₂ aryl, C₇-C₂₄ alkaryl, C₇-C₂₄ aralkyl, C₁₋₄ halo substituted alkyl up to per halo alkyl, C₆-C₁₂ halo substituted aryl up to per halo aryl, C₃-C₁₂ halo substituted cycloalkyl having 0-3 heteroatoms selected from N, S and O, up to per halo cycloalkyl, halo substituted C₃-C₁₂ hetaryl up to per halo heteraryl, halo substituted C₇-C₂₄ aralkyl up to per halo aralkyl, halo substituted C₇-C₂₄ alkaryl up to per halo alkaryl, and —C(O)R_{g},
      where R_{g} is C₁₋₁₀ alkyl; —CN, —CO₂R_{d}, —OR_{d}, —SR_{d}, —NO₂, C(O)Rₑ, —NR_{d}Rₑ, —NR_{d}C(O)ORₑ and —NR_{d}(CO)Rₑ and R_{d} and Rₑ are independently selected from the group consisting of hydrogen, C₁₋₁₀ alkyl, C₁₋₁₀ alkoxy, C₃₋₁₀ cycloalkyl having 0-3 heteroatoms selected from O, N and S, C₆₋₁₂ aryl, C₃-C₁₂ hetaryl with 1-3 heteroatoms selected from O, N and S and C₇-C₂₄ aralkyl, C₇-C₂₄ alkaryl, up to per halo substituted C₁-C₁₀ alkyl, up to per halo substituted C₃-C₁₀ cycloalkyl having 0-3 heteroatoms selected from O, N and S, up to per halo substituted C₆-C₁₄ aryl, up to per halo substituted C₃-C₁₂ hetaryl having 1-3 heteroatoms selected from O, N and S, halo substituted C₇-C₂₄ alkaryl up to per halo alkaryl, and up to per halo substituted C₇-C₂₄ aralkyl,
W is independently selected from the group consisting CN, —CO₂R⁵, —C(O)NR⁵R⁵, —C(O)—R⁵, —NO₂, —OR⁵, —SR⁵, —NR⁵R⁵, —NR⁵C(O)OR⁵, —NR⁵C(O)R⁵, C₁-C₁₀ alkyl, C₁-C₁₀ alkoxy, C₂-C₁₀ alkenyl, C₁-C₁₀ alkenoyl, C₃-C₁₀ cycloalkyl having 0-3 heteroatoms selected from O, S and N, C₆-C₁₄ aryl, C₇-C₂₄ alkaryl, C₇-C₂₄ aralkyl, C₃-C₁₂ heteroaryl having 1-3 heteroatoms selected form O, N and S, C₄-C₂₃ alkheteroaryl having 1-3 heteroatoms selected from O, N and S, substituted C₁-C₁₀ alkyl, substituted C₁-C₁₀ alkoxy, substituted C₂-C₁₀ alkenyl, substituted C₁-C₁₀ alkenoyl, substituted C₃-C₁₀ cycloalkyl having 0-3 heteroatoms selected from O, N and S, substituted C₆-C₁₂ aryl, substituted C₃-C₁₂ hetaryl having 1-3 heteroatoms selected from O, N and S, substituted C₇-C₂₄ aralkyl, substituted C₇-C₂₄ alkaryl, substituted C₄-C₂₃ alkheteroaryl having 1-3 heteroatoms selected from O, N and S, and -Q-Ar;
R⁵ is independently selected from H, C₁-C₁₀ alkyl, C₁-C₁₀ alkoxy, C₂-C₁₀ alkenyl, C₁-C₁₀ alkenoyl, C₃-C₁₀ cycloalkyl having 0-3 heteroatoms selected from O, C and N, C₆-C₁₄ aryl, C₃-C₁₃ hetaryl having 1-3 heteroatoms selected from O, N and S, C₇-C₁₄ alkaryl, C₇-C₂₄ aralkyl; C₄-C₂₃ alkheteroaryl having 1-3 heteroatoms selected from O, N and S, up to per-halosubstituted C₁-C₁₀ alkyl, up to per-halosubstituted C₃-C₁₀ cycloalkyl having 0-3 heteroatoms selected from O, N and S, up to per-halosubstituted C₆-C₁₄ aryl, up to per-halosubstituted C₃-C₁₃ hetaryl having 1-3 heteroatoms selected from O, N and S, up to per-halosubstituted C₇-C₂₄ aralkyl, up to per-halosubstituted C₇-C₂₄ alkaryl, and up to per-halosubstituted C₄-C₂₃ alkheteroaryl; and each Z is independently selected from the group consisting —CN, —CO₂R⁵, —C(O)NR⁵R⁵, —C(O)—R⁵, —NO₂, —OR⁵, —SR⁵, —NR⁵R⁵, —NR⁵C(O)OR⁵, —NR⁵C(O)R⁵, C₁-C₁₀ alkyl, C₁-C₁₀ alkoxy, C₂-C₁₀ alkenyl, C₁-C₁₀ alkenoyl, C₃-C₁₀ cycloalkyl having 0-3 heteroatoms selected from O, S and N, C₆-C₁₄ aryl, C₇-C₂₄ alkaryl, C₇-C₂₄ aralkyl, C₃-C₁₂ heteroaryl having 1-3 heteroatoms selected from O, N and S, C₄-C₂₃ alkheteroaryl having 1-3 heteroatoms selected from O, N and S, substituted C₁-C₁₀ alkyl, substituted C₁-C₁₀ alkoxy, substituted C₂-C₁₀ alkenyl, substituted C₁-C₁₀ alkenoyl, substituted C₃-C₁₀ cycloalkyl having 0-3 heteroatoms selected from O, N and S, substituted C₆-C₁₂ aryl, substituted C₃-C₁₂ hetaryl having 1-3 heteroatoms selected from O, N and S; wherein if Z is a substituted group, the one or more substituents are selected from the group consisting of —CN, —CO₂R⁵, —C(O)NR⁵R⁵, —C(O)—R⁵, —NO₂, —OR⁵, —SR⁵, —NR⁵R⁵, —NR⁵C(O)OR⁵, —NR⁵C(O)R⁵.

According to the invention, each M independently from one another represents a bond or is a bridging group, selected from the group consisting of (CR⁵R⁵)ₕ, or (CHR⁵)ₕ-Q-(CHR⁵)ᵢ, wherein
Q is selected from a group consisting of O, S, N—R⁵, (CHal₂)ⱼ, (O—CHR⁵)ⱼ, (CHR⁵—O)ⱼ, CR⁵═CR⁵, (O—CHR⁵CHR⁵)ⱼ, (CHR⁵CHR⁵—O)ⱼ, C═O, C═S, C═NR⁵, CH(OR⁵), C(OR⁵)(OR⁵), C(═O)O, OC(═O), OC(═O)O, C(═O)N(R⁵), N(R⁵)C(═O), OC(═O)N(R⁵), N(R⁵)C(═O)O, CH═N—O, CH═N—NR⁵, OC(O)NR⁵, NR⁵C(O)O, S═O, SO₂, SO₂NR⁵ and NR⁵SO₂, wherein
R⁵ is in each case independently selected from the meanings given above, preferably from hydrogen, halogen, alkyl, aryl, aralkyl,
h, i are independently from each other 0, 1, 2, 3, 4, 5 or 6, preferably 0, 1, 2, or 3, and
j is 1, 2, 3, 4, 5 or 6, preferably 0, 1, 2 or 3.

More preferred, each M independently from one another represents a bond or is a bridging group, selected from the group consisting of —O—, —S—, —N(R⁵)—, —(CH₂)_{β}—, —C(O)—, —CH(OH)—, —(CH₂)_{β}O—, —(CH₂)_{β}S—, —(CH₂)_{β}N(R⁵)—, —O(CH₂)_{β}, —CHHal-, —CHal₂-, —S—(CH₂)_{β}— and —N(R⁵)(CH₂)_{β}, where, β is 1 to 6 and especially preferred 1 to 3, Hal is halogen and R⁵ is as defined above. More preferred, the group B of Formula I is a substituted or unsubstituted six member aryl moiety or six member hetaryl moiety, said hetaryl moiety having 1 to 4 members selected from the group of hetaryl atoms consisting of nitrogen, oxygen and sulfur with the balance of the hetaryl moiety being carbon.

Even more preferred, the group B of Formula I is
a) an unsubstituted phenyl group, an unsubstituted pyridyl group, an unsubstituted pyrimidinyl, an unsubstituted indole group, a phenyl group substituted by a substituent selected from the group consisting of halogen and Wγ wherein W and γ are as defined in claim 1, a pyrimidinyl group substituted by a substituent selected from the group constituting of halogen and Wγ, whereas W and γ are as defined above, or a substituted pyridyl group, substituted by a substituent selected from the group consisting of halogen and Wγ wherein W and γ are as defined above; or a substituted phenyl group, a substituted pyrimidinyl group, or substituted pyridyl group substituted 1 to 3 times by 1 or more substituents selected from the group consisting of —CN, halogen, C₁-C₁₀ alkyl, C₁-C₁₀ alkyl alkoxy, —OH, up to per halo substituted C₁-C₁₀ alkyl, up to per halo substituted C₁-C₁₀ alkoxy or phenyl substituted by halogen up to per halo; or
b) a substituted phenyl group, a substituted pyrimidinyl group, or substituted pyridyl group substituted 1 to 3 times b 1 or more substituents selected from the group consisting of —CN, halogen, alkyl, especially C₁-C₄ alkyl, alkoxy, especially C₁-C₄ alkoxy, —OH, acyl, especially acetyl, up to per halo substituted alkyl, especially up to per halo substituted C₁-C₄ alkyl, up to per halo substituted alkoxy, especially up to per halo substituted C₁-C₄ alkoxy or phenyl substituted by halogen up to per halo or a substituted indole group.

In the formula I, the group L which is directly bound to D is preferably a substituted or unsubstituted 6 member aryl moiety or a substituted or unsubstituted 6 member hetaryl moiety, wherein said hetaryl moiety has 1 to 4 members selected from the group of heteroatoms consisting of nitrogen, oxygen and sulfur with the balance of said hetaryl moiety being carbon, wherein the one or more substituents are selected from the group consisting of halogen and Wγ wherein W and γ are as defined above.

More preferred, the group L is a substituted phenyl, unsubstituted phenyl, substituted pyrimidinyl, unsubstituted pyrimidinyl, substituted pyridyl or unsubstituted pyridyl group.

In the formula I, the group L′ preferably comprises a 5 to 6 membered aryl moiety or hetaryl moiety, wherein said heteraryl moiety comprises 1 to 4 members selected from the group of heteroatoms consisting of nitrogen, oxygen and sulfur.

More preferred, the group L′ is phenyl, pyridinyl, pyrimidinyl, or pyrrolyl.

Hence, preferred compounds of formula I are of formula Ia
A-CO—NH—NH—CO—B Ia
wherein A and B are as defined above/below, and wherein the carbonyl moieties in formula Ia can be derivatized as described above/below, and the salts or solvates thereof. Especially preferred are compounds of formula Ia, wherein the carbonyl moiety is not derivatized.

Preferably, A or B is substituted by one or more substituents as described above/below. More preferably, A and B each are substituted by one or more substituents as described above/below. Even more preferably, A is substituted by two or more substituents as described above/below.

Preferably, subject of the present invention are the optically active forms or stereo isomers of the compounds according to the invention, such as the enantiomers, the diastereomers and/or mixtures thereof in all ratios, such as, for example, stereochemically uniform compounds or racemates. Preferably, further subject of the present invention are the solvates and hydrates of the compounds according to the invention. Preferably, further subject of the present invention are the pharmaceutically acceptable derivatives or physiologically functional derivatives of the compounds according to the invention. More preferably, further subject of the present invention are the salts of the compounds according to the invention, especially the pharmaceutically and/or physiologically acceptable salts of compounds according to the invention.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “alkyl” preferably refers to a straight or branched chain hydrocarbon having from one to twelve carbon atoms, optionally substituted with substituents selected from the group consisting of C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or C₁-C₆ perfluoroalkyl, multiple degrees of substitution being allowed. Examples of “alkyl” as used herein include, but are not limited to, methyl, ethyl, n-propyl, isopropyl, n-butyl, isobutyl, t-butyl, n-pentyl, isopentyl, and the like.

As used herein, the term “C₁-C₆ alkyl” preferably refers to an alkyl group as defined above containing at least 1, and at most 6, carbon atoms. Examples of branched or straight chained “C₁-C₆ alkyl” groups useful in the present invention include, but are not limited to, methyl, ethyl, n-propyl, isopropyl, isobutyl, n-butyl, t-butyl, n-pentyl and isopentyl.

As used herein, the term “alkylene” preferably refers to a straight or branched chain divalent hydrocarbon radical having from one to ten carbon atoms, optionally substituted with substituents selected from the group which includes lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl, optionally substituted by alkyl, nitro, cyano, halogen and lower perfluoroalkyl, multiple degrees of substitution being allowed. Examples of “alkylene” as used herein include, but are not limited to, methylene, ethylene, n-propylene, n-butylene and the like.

As used herein, the term “C₁-C₆ alkylene” preferably refers to an alkylene group, as defined above, which contains at least 1, and at most 6, carbon atoms respectively. Examples of “C₁-C₆ alkylene” groups useful in the present invention include, but are not limited to, methylene, ethylene and n-Propylene.

As used herein, the term “halogen” or “hal” preferably refers to fluorine (F), chlorine (Cl), bromine (Br) or iodine (I).

As used herein, the term “C₁-C₆ haloalkyl” preferably refers to an alkyl group as defined above containing at least 1, and at most 6, carbon atoms substituted with at least one halogen, halogen being as defined herein. Examples of branched or straight chained “C₁-C₆ haloalkyl” groups useful in the present invention include, but are not limited to, methyl, ethyl, propyl, isopropyl, isobutyl and n-butyl substituted independently with one or more halogens, e.g., fluoro, chloro, bromo and iodo.

As used herein, the term “C₃-C₇ cycloalkyl” preferably refers to a non-aromatic cyclic hydrocarbon ring having from three to seven carbon atoms and which optionally includes a C₁-C₆ alkyl linker through which it may be attached. The C₁-C₆ alkyl group is as defined above. Exemplary “C₃-C₇ cycloalkyl” groups include, but are not limited to, cyclopropyl, cyclobutyl, cyclopentyl, cyclohexyl and cycloheptyl. The term “cycloalkyl”, as used herein preferably also includes saturated heterocyclic groups, which are preferably selected from the cycloalkyl- groups as defined above, wherein one or two carbon atoms are replaced by hetero atoms, selected from the group consisting of O, N and S.

As used herein, the term “C₃-C₇ cycloalkylene” preferably refers to a non-aromatic alicyclic divalent hydrocarbon radical having from three to seven carbon atoms, optionally substituted with substituents selected from the group which includes lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, lower perfluoroalkyl, multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, or cyclooctyl-1,5-diyl, and the like.

As used herein, the term “heterocyclic” or the term “heterocyclyl” preferably refers to a three to twelve-membered heterocyclic ring having one or more degrees of unsaturation containing one or more heteroatomic substitutions selected from S, SO, SO₂, O or N, optionally substituted with substituents selected from the group consisting of C₁-C₆ alkyl, C₁-C₆ haloalkyl, C₁-C₆ alkoxy, C₁-C₆ alkylsulfanyl, C₁-C₆ haloalkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or C₁-C₆ perfluoroalkyl, multiple degrees of substitution being allowed. Such a ring may be optionally fused to one or more other “heterocyclic” ring(s) or cycloalkyl ring(s). Examples of “heterocyclic” moieties include, but are not limited to, tetrahydrofuran, pyran, 1,4-dioxane, 1,3-dioxane, pyrrolidine, piperidine, morpholine, tetrahydrothiopyran, tetrahydrothiophene, and the like.

As used herein, the term “heterocyclylene” preferably refers to a three to twelve-membered heterocyclic ring diradical having one or more degrees of unsaturation containing one or more heteroatoms selected from S, SO, SO₂, O or N, optionally substituted with substituents selected from the group which includes lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, lower perfluoroalkyl, multiple degrees of substitution being allowed. Such a ring may be optionally fused to one or more benzene rings or to one or more of another “heterocyclic” rings or cycloalkyl rings. Examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl, and the like.

As used herein, the term “aryl” preferably refers to an optionally substituted benzene ring or to an optionally substituted benzene ring system fused to one or more optionally substituted benzene rings to form, for example, anthracene, phenanthrene, or napthalene ring systems. Exemplary optional substituents include C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, C₁-C₆ perfluoroalkyl, heteroaryl, or aryl, multiple degrees of substitution being allowed. Examples of “aryl” groups include, but are not limited to Phenyl, 2-naphthyl, 1-naphthyl, biphenyl, as well as substituted derivatives thereof.

As used herein, the term “arylene” preferably refers to a benzene ring diradical or to a benzene ring system diradical fused to one or more optionally substituted benzene rings, optionally substituted with substituents selected from the group which includes lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, lower perfluoroalkyl, heteroaryl and aryl, multiple degrees of substitution being allowed. Examples of “arylene” include, but are not limited to benzene-1,4-diyl, naphthalene-1,8-diyl, anthracene-1,4-diyl, and the like.

As used herein, the term “aralkyl” preferably refers to an aryl or heteroaryl group, as defined herein, attached through a C₁-C₆ alkyl linker, wherein C₁-C₆ alkyl is as defined herein. Examples of “aralkyl” include, but are not limited to, benzyl, phenylpropyl, 2-pyridylmethyl, 3-isoxazolylmethyl, 5-methyl-3-isoxazolylmethyl and 2-imidazolylethyl.

As used herein, the term “heteroaryl” preferably refers to a monocyclic five to seven-membered aromatic ring, or to a fused bicyclic aromatic ring system comprising at least one of such monocyclic five to seven-membered aromatic rings and at least one aromatic or non-aromatic five to seven-membered ring. These heteroaryl rings contain one or more nitrogen, sulfur and/or oxygen heteroatoms, where N-Oxides and sulfur Oxides and dioxides are permissible heteroatom substitutions and may be optionally substituted with up to three members selected from a group consisting of C₁-C₆ alkyl, C₁-C₆ haloalkyl, C₁-C₆ alkoxy, C₁-C₆ alkylsulfanyl, C₁-C₆ haloalkylsulfanyl, C₁-C₆ alkylsulfenyl, C₁-C₆ alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, C₁-C₆ perfluoroalkyl, heteroaryl or aryl, multiple degrees of substitution being allowed. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, benzofuranyl, benzothiophenyl, indolyl, indazolyl, benzimidazolyl, benzopyrazolyl, benzoxazolyl, benzisoxazolyl, benzothiazolyl, benzisothiazolyl, benzoxadiazolyl, benzooxazinyl, benzodioxolyl, benzodioxanyl, benzoxadiazolyl, benzotriazolyl, benzooxazinyl, dihydrobenzofuranyl, dihydrobenzoisofuranyl, chromanyl, isochromanyl, benzodioxinyl, benzodioxolyl, benzothiadiazolyl and substituted versions thereof.

As used herein, the term “heteroarylene” preferably refers to a five- to seven-membered aromatic ring diradical, or to a polycyclic heterocyclic aromatic ring diradical, containing one or more nitrogen, oxygen, or sulfur heteroatoms, where N-Oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, lower perfluoroalkyl, heteroaryl, or aryl, multiple degrees of substitution being allowed. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4-thiadiazole-2,5-diyl, 1,3-thiazole-2,5-diyl, pyridine-2,4-diyl, pyridine-2,3-diyl, pyridine-2,5-diyl, pyrimidine-2,4-diyl, quinoline-2,3-diyl, and the like.

As used herein, the term “alkoxy” preferably refers to the group RₐO—, where Rₐ is alkyl as defined above and the term “C₁-C₆ alkoxy” preferably refers to an alkoxy group as defined herein wherein the alkyl moiety contains at least 1 and at most 6 carbon atoms. Exemplary C₁-C₆ alkoxy groups useful in the present invention include, but are not limited to methoxy, ethoxy, n-propoxy, isopropoxy, n-butoxy and t-butoxy.

As used herein, the term “haloalkoxy” preferably refers to the group RₐO—, where Rₐ is haloalkyl as defined above and the term “C₁-C₆ haloalkoxy” preferably refers to an haloalkoxy group as defined herein wherein the haloalkyl moiety contains at least 1 and at most 6 carbon atoms. Exemplary C₁-C₆ haloalkoxy groups useful in the present invention include, but are not limited to, methoxy, ethoxy, n-propoxy, isopropoxy, n-butoxy and t-butoxy substituted with one or more halo groups, for instance trifluoromethoxy.

As used herein the term “aralkoxy” preferably refers to the group R_{C}R_{B}O—, where R_{B} is alkyl and R_{C} is aryl as defined above.

As used herein the term “aryloxy” preferably refers to the group R_{C}O—, where R_{C} is aryl as defined above.

As used herein, the term “alkylsulfanyl” preferably refers to the group R_{A}S—, where R_{A} is alkyl as defined above and the term “C₁-C₆ alkylsulfanyl” preferably refers to an alkylsulfanyl group as defined herein wherein the alkyl moiety contains at least 1 and at most 6 carbon atoms.

As used herein, the term “haloalkylsulfanyl” preferably refers to the group R_{D}S—, where R_{D} is haloalkyl as defined above and the term “C₁-C₆ haloalkylsulfanyl” preferably refers to a haloalkylsulfanyl group as defined herein wherein the alkyl moiety contains at least 1 and at most 6 carbon atoms.

As used herein, the term “alkylsulfenyl” preferably refers to the group R_{A}S(O)—, where R_{A} is alkyl as defined above and the term “C₁-C₆ alkylsulfenyl” preferably refers to an alkylsulfenyl group as defined herein wherein the alkyl moiety contains at least 1 and at most 6 carbon atoms.

As used herein, the term “alkylsulfonyl” preferably refers to the group R_{A}SO₂—, where R_{A} is alkyl as defined above and the term “C₁-C₆ alkylsulfonyl” preferably refers to an alkylsulfonyl group as defined herein wherein the alkyl moiety contains at least 1 and at most 6 carbon atoms.

As used herein, the term “oxo” preferably refers to the group ═O.

As used herein, the term “mercapto” preferably refers to the group —H.

As used herein, the term “carboxy” preferably refers to the group —COH.

As used herein, the term “cyano” preferably refers to the group —CN.

As used herein, the term “cyanoalkyl” preferably refers to the group —R_{B}CN, wherein R_{B} is alkylene as defined above. Exemplary “cyanoalkyl” groups useful in the present invention include, but are not limited to, cyanomethyl, cyanoethyl and cyanoisopropyl.

As used herein, the term “aminosulfonyl” preferably refers to the group —SO₂NH₂.

As used herein, the term “carbamoyl” preferably refers to the group —C(O)NH₂.

As used herein, the term “sulfanyl” shall refer to the group —S—.

As used herein, the term “sulfenyl” shall refer to the group —S(O)—.

As used herein, the term “sulfonyl” shall refer to the group —S(O)₂— or —SO₂.

As used herein, the term “acyl” preferably refers to the group R_{F}C(O)—, where R_{F} is alkyl, cycloalkyl or heterocyclyl as defined herein.

As used herein, the term “aroyl” preferably refers to the group R_{C}C(O)—, where R_{c} is aryl as defined herein.

As used herein, the term “heteroaroyl” preferably refers to the group R_{E}C(O), where R_{E} is heteroaryl as defined herein.

As used herein, the term “alkoxycarbonyl” preferably refers to the group R_{A}OC(O)—, where R_{A} is alkyl as defined herein.

As used herein, the term “acyloxy” preferably refers to the group R_{F}C(O)O—, where R_{F} is alkyl, cycloalkyl, or heterocyclyl as defined herein.

As used herein, the term “aroyloxy” preferably refers to the group R_{C}C(O)O—, where R_{c} is aryl as defined herein.

As used herein, the term “heteroaroyloxy” preferably refers to the group R_{E}C(O)O—, where R_{E} is heteroaryl as defined herein.

As used herein, the term “carbonyl” or “carbonyl moiety” preferably refers to the group C═O.

As used herein, the term “thiocarbonyl” or “thiocarbonyl moiety” preferably refers to the group C═S.

As used herein, the term “amino”, “amino group” or “imino moiety” preferably refers to the group NR_{G}R_{G′}, wherein R_{G} and R_{G′}, are preferably selected, independently from one another, from the group consisting of hydrogen, halogen, alkyl, haloalkyl, alkenyl, cycloalkyl, alkylenecycloalkyl, cyanoalkyl, aryl, aralkyl, heteroaryl, acyl and aroyl. If both R_{G} and R_{G′} are hydrogen, NR_{G}R_{G′} is also referred to as “unsubstituted amino moiety” or “unsubstituted amino group”. If R_{G} and/or R_{G′} are other than hydrogen, NR_{G}R_{G′} is also referred to as “substituted amino moiety” or “substituted amino group”.

As used herein, the term “imino” or “imino moiety” preferably refers to the group C═NR_{G}, wherein R_{G} is preferably selected from the group consisting of hydrogen, halogen, alkyl, haloalkyl, alkenyl, cycloalkyl, alkylenecycloalkyl, cyanoalkyl, aryl, aralkyl, heteroaryl, acyl and aroyl. If R_{G} is hydrogen, C═NR_{G} is also referred to as “unsubstituted imino moiety”. If R_{G} is a residue other than hydrogen, C═NR_{G} is also referred to as “substituted imino moiety”.

As used herein, the term “ethene-1,1-diyl moiety” preferably refers to the group C═CR_{K}R_{L}, wherein R_{K} and R_{L} are preferably selected, independently from one another, from the group consisting of hydrogen, halogen, alkyl, haloalkyl, alkenyl, cycloalkyl, nitro, alkylenecycloalkyl, cyanoalkyl, aryl, aralkyl, heteroaryl, acyl and aroyl. If both hydrogen R_{K} and R_{L} are hydrogen, C═CR_{K}R_{L} is also referred to as “unsubstituted ethene-1,1-diyl moiety”. If one of R_{K} and R_{L} or both are a residue other than hydrogen, C═CR_{K}R_{L} is also referred to as “substituted ethene-1,1-diyl moiety”.

As used herein, the terms “group”, “residue” and “radical” or “groups”, “residues” and “radicals” are usually used as synonyms, respectively, as it is common practice in the art.

As used herein, the term “optionally” means that the subsequently described event(s) may or may not occur, and includes both event(s), which occur, and events that do not occur.

As used herein, the term “pharmaceutically acceptable derivative” preferably refers to any physiologically functional derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives. Such derivatives preferably include so-called prodrug-compounds, for example compounds according to the invention that are derivatized with alkyl groups, acyl groups, sugars or peptides, such as oligopeptides, and that are easily degraded or metabolized to the active compounds according to the invention. Such derivatives preferably include biodegradable polymer derivatives of the compounds according to the invention. Suitable polymers and methods for producing biodegradable polymeric derivatives are known in the art, for example from Int. J. Pharm. 115, 61-67 (1995).

As used herein, the term “solvate” preferably refers to a complex of variable stoichiometry formed by a solute (in this invention, a compound of formula I or formula II or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water. Examples for suitable solvates are the mono- or dihydrates or alcoholates of the compounds according to the invention.

As used herein, the term “substituted” preferably refers to substitution with the named substituent or substituents, multiple degrees of substitution being allowed unless otherwise stated.

Certain of the compounds described herein may contain one or more chiral atoms, or may otherwise be capable of existing as two or more stereoisomers, which are usually enantiomers and/or diastereomers. Accordingly, the compounds of this invention include mixtures of stereoisomers, especially mixtures of enantiomers, as well as purified stereoisomers, especially purified enantiomers, or stereoisomerically enriched mixtures, especially enantiomerically enriched mixtures. Also included within the scope of the invention are the individual isomers of the compounds represented by formulae I and II above as well as any wholly or partially equilibrated mixtures thereof. The present invention also covers the individual isomers of the compounds represented by the formulas above as mixtures with isomers thereof in which one or more chiral Centers are inverted. Also, it is understood that all tautomers and mixtures of tautomers of the compounds of formulae (I) or (II) are included within the scope of the compounds of formulae (I) and (II) and preferably the formulae and subformulae corresponding thereto.

Racemates obtained can be resolved into the isomers mechanically or chemically by methods known per se. Diastereomers are preferably formed from the racemic mixture by reaction with an optically active resolving agent. Examples of suitable resolving agents are optically active acids, such as the D and L forms of tartaric acid, diacetyltartaric acid, dibenzoyltartaric acid, mandelic acid, malic acid, lactic acid or the various optically active camphorsulfonic acids, such as p-camphorsulfonic acid. Also advantageous is enantiomer resolution with the aid of a column filled with an optically active resolving agent (for example dinitrobenzoylphenylglycine); an example of a suitable eluent is a hexane/isopropanol/acetonitrile mixture.

The diastereomer resolution can also be carried out by standard purification processes, such as, for example, chromatography or fractional crystallization.

It is of course also possible to obtain optically active compounds of the formula I or II by the methods described above by using starting materials which are already optically active.

Unless indicated otherwise, it is to be understood that reference to compounds of formula I preferably includes the reference to the compounds of formula II. Unless indicated otherwise, it is to be understood that reference to the compounds of formula II preferably includes the reference to the sub formulae corresponding thereto, for example the sub formulae II.1 to II.11 and preferably formulae IIa to IIx. It is also understood that the following embodiments, including uses and compositions, although recited with respect to formula I are preferably also applicable to formulae II, sub formulae II1 to II.11 and preferably formulae IIa to IIx.

Especially preferred compounds according to the invention are compounds of formula II 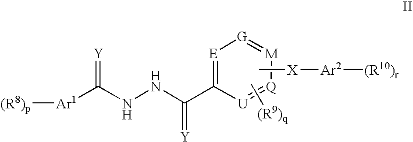
wherein
Ar¹, Ar² are selected independently from one another from aromatic hydrocarbons containing 6 to 14 carbon atoms and ethylenical unsaturated or aromatic heterocyclic residues containing 3 to 10 carbon atoms and one, two or three heteroatoms, independently selected from N, O and S,
E, G, M, Q
and U are selected, independently from one another, from carbon atoms and nitrogen atoms, with the proviso that one or more of E, G, M. Q and U are carbon atoms and that X is bonded to a carbon atom,
R⁸, R⁹ and R¹⁰ are independently selected from a group consisting of H, A, A, OA, cycloalkyl comprising 3 to 7 carbon atoms, Hal, CH₂Hal, CH(Hal)₂, C(Hal)₃, NO₂, (CH₂)ₙCN, (CH₂)ₙNR¹¹“R², (CH₂)ₙO(CH₂)ₖNR¹¹R¹², (CH₂)ₙNR¹¹(CH₂)ₖNR¹¹R¹², (CH₂)ₙO(CH₂)ₖOR¹¹, (CH₂)ₙNR¹¹(CH₂)ₖOR², (CH₂)ₙCOOR¹³ (CH₂)ₙCOR¹³, (CH₂)ₙCONR¹¹R¹², (CH₂)ₙNR¹¹COR¹³ (CH₂)ₙNR¹¹CONR¹¹R¹², (CH₂)ₙNR¹¹SO₂A, (CH₂)ₙSO₂NR¹¹R¹², (CH₂)ₙS(O)ᵤR¹³, (CH₂)ₙOC(O)R¹³ (CH₂)ₙCOR¹³, (CH₂)ₙSR¹¹, CH═N-OA, CH₂CH═N-OA, (CH₂)ₙNHOA, (CH₂)ₙCH═N—R¹¹, (CH₂)ₙOC(O)NR¹¹R¹², (CH₂)ₙNR¹¹COOR¹³, (CH₂)ₙN(R¹¹)CH₂CH₂OR¹³ (CH₂)ₙN(R¹¹)CH₂CH₂OCF₃, (CH₂)ₙN(R¹¹)C(R¹³)HCOOR¹², (CH₂)ₙN(R¹¹)C(R¹³)HCOR¹¹, (CH₂)ₙN(R¹¹)CH₂CH₂N(R¹²)CH₂COOR¹¹, (CH₂)ₙN(R¹¹)CH₂CH₂NR¹¹R¹², CH═CHCOOR¹³, CH═CHCH₂NR¹¹R¹², CH═CHCH₂NR¹¹R¹², CH═CHCH₂OR³, (CH₂)ₙN(COOR¹³)COOR¹⁴, (CH₂)ₙN(CON H₂)COOR¹³, (CH₂)ₙN(CONH₂)CONH₂, (CH₂)ₙN(CH₂COOR¹³)COOR¹⁴, (CH₂)ₙN(CH₂CONH₂)COOR¹³, (CH₂)ₙN(CH₂CONH₂)CONH₂, (CH₂)ₙCHR¹³COR¹⁴, (CH₂)ₙCHR³COOR⁴, (CH₂)ₙCHR¹³CH₂OR¹⁴, (CH₂)ₙOCN and (CH₂)ₙNCO, wherein
R¹¹, R¹² are independently selected from a group consisting of H, A, (CH₂)ₘAr³ and (CH₂)ₘHet, or in NR¹¹R¹²,
R¹¹ and R¹² form, together with the N-atom they are bound to, a 5-, 6- or 7-membered heterocycles which optionally contains 1 or 2 additional hetero atoms, selected from N, O and S,
R¹³, R¹⁴ are independently selected from a group consisting of H, Hal, A, (CH₂)^{m}Ar⁴ and (CH₂)ₘHet,
A is selected from the group consisting of alkyl, alkenyl, cycloalkyl, alkylenecycloalkyl, alkoxy, alkoxyalkyl and saturated heterocyclyl, preferably from the group consisting of alkyl, alkenyl, cycloalkyl, alkylenecycloalkyl, alkoxy and alkoxyalkyl,
Ar³, Ar⁴ are independently from one another aromatic hydrocarbon residues comprising 5 to 12 and preferably 5 to 10 carbon atoms which are optionally substituted by one or more substituents, selected from a group consisting of A, Hal, NO₂, CN, OR¹⁵, NR¹⁵R¹⁶, COOR¹⁵, CONR¹⁵R¹⁶, NR¹⁵COR¹⁶, NR¹⁵CONR¹⁵R¹⁶, NR¹⁶SO₂A, COR¹⁵, SO₂R¹⁵R¹⁶, S(O)ᵤA and OOCR¹⁵,
Het is a saturated, unsaturated or aromatic heterocyclic residue which is optionally substituted by one or more substituents, selected from a group consisting of A, Hal, NO₂, CN, OR¹⁵, NR¹⁵R¹⁶, COOR¹⁵, CONR¹⁵R¹⁶, NR¹⁵COR¹⁶, NR¹⁵CONR¹⁵R¹⁶, NR¹⁶SO₂A, COR¹⁵, SO₂R¹⁵R¹⁶, S(O)ₙA and OOCR¹⁵,
R¹⁵, R¹⁶ are independently selected from a group consisting of H, A, and (CH₂)ₘAr⁶, wherein
Ar⁶ is a 5- or 6-membered aromatic hydrocarbon which is optionally substituted by one or more substituents selected from a group consisting of methyl, ethyl, propyl, 2-propyl, tert.-butyl, Hal, —CN, OH, —NH₂ and CF₃,
k, n and m are independently of one another 0, 1, 2, 3, 4, or 5,
X represents a bond or is (CR¹¹R¹²)ₕ, or (CHR¹¹)ₕ-Q-(CHR¹²)ᵢ, wherein
Q is selected from a group consisting of T, CH¹⁵H¹⁶, N—R¹⁵, (CHal₂)ⱼ, (O—CHR¹⁸)ⱼ, (CHR¹⁸—O)ⱼ, CR¹⁸═CR¹⁹, (O—CHR⁸CHR¹⁹)ⱼ, (CHR¹⁸CHR¹⁹—O)ⱼ, C═O, C═S, C═NR¹⁵, CH(OR¹⁵), C(OR¹⁵)(OR²⁰), C(═O)O, OC(═O), OC(═O)O, C(═O)N(R¹⁵), N(R¹⁵)C(═O), OC(═O)N(R¹⁵), N(R¹⁵)C(═O)O, CH═N—O, CH═N—NR¹⁵, OC(O)NR¹⁵, NR¹⁵C(O)O, S═O, SO₂, SO₂NR¹⁵ and NR¹⁵SO₂, wherein
T is selected from O, S, N—R¹⁵,
h, i are independently from each other 0, 1, 2, 3, 4, 5, or 6, and
j is 1, 2, 3, 4, 5, or 6,
Y is selected from O/S, NR²¹, C(R²²)—NO₂, C(R²²)—CN and C(CN)₂, wherein
O/S is selected from O, S,
R²¹ is independently selected from the meanings given for R¹³, R¹⁴ and
R²² is independently selected from the meanings given for R¹¹, R¹²,
p, r are independently from one another 0, 1, 2, 3, 4 or 5,
q is 0, 1, 2, 3 or 4, preferably 0, 1 or 2,
u is 0, 1, 2 or 3, preferably 0, 1 or 2,
and
Hal is independently selected from a group consisting of F, Cl, Br and I, preferably F, Cl and Br;
   and the pharmaceutically acceptable derivatives, solvates, salts and stereoisomers thereof, including mixtures thereof in all ratios, and more preferred the salts and/or solvates thereof, and especially preferred the physiologically acceptable salts and/or solvates thereof.

Subject of the present invention are especially compounds of formula I and II, in which one or more substituents or groups, preferably the major part of the substituents or groups has a meaning which is indicated as preferred, more preferred or especially preferred.

In compounds of formula II, E, G, M, Q and U constitute, together with the carbon atom that E and U are bound to, a bivalent 6-membered aromatic or nitrogen containing heteroaromatic ring. Preferably, one or more of E, G, M, Q and U, more preferably two or more of E, G, M, Q and U and especially three or more of E, G, M, Q and U are carbon atoms. Especially preferred, none or one of E, G, M, Q and U is a nitrogen atom. Especially preferred, E, G, M, Q and U constitute, together with the carbon atom that E and U are bound to, a 6-membered aromatic or nitrogen containing heteroaromatic ring, selected from the group consisting of phenylen, pyridinylen and pyrimydylen, wherein X is preferably bonded to a carbon atom. The substituents R⁹ are preferably bound to a carbon atom.

Especially preferred as compounds of formula II are compounds of formula II′, 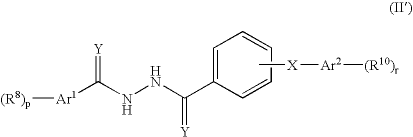
wherein E, G, M, Q and U of formula II are carbon atoms and R⁹ of formula II is H.

In compounds of formula II, the term alkyl preferably refers to an unbranched or branched alkyl residue, preferably an unbranched alkyl residue comprising 1, 2, 3, 4, 5, 6, 7, 8, 9 or 10, preferably 1, 2, 3, 4, 5 or 6, more preferred 1, 2, 3 or 4 and especially 1 or 2 carbon atoms, or a branched alkyl residue comprising 3, 4, 5, 6, 7, 8, 9 or 10, preferably 3, 4, 5 or 6 more preferred 3 or 4 carbon atoms. The alkyl residues can be optionally substituted, especially by one or more halogen atoms, for example up to perhaloalkyl, by one or more hydroxy groups or by one or more amino groups, all of which can optionally be substituted by alkyl. If an alkyl residue is substituted by halogen, it usually comprises 1, 2, 3, 4 or 5 halogen atoms, depending on the number of carbon atoms of the alkyl residue. For example, a methyl group can comprise, 1, 2 or 3 halogen atoms, an ethyl group (an alkyl residue comprising 2 carbon atoms) can comprise 1, 2, 3, 4 or 5 halogen atoms. If an alkyl residue is substituted by hydroxy groups, it usually comprises one or two, preferably one hydroxy groups. If the hydroxy group is substituted by alkyl, the alkyl substituent comprises preferably 1 to 4 carbon atoms and is preferably unsubstituted or substituted by halogen and more preferred unsubstituted. If an alkyl residue is substituted by amino groups, it usually comprises one or two, preferably one amino groups. If the amino group is substituted by alkyl, the alkyl substituent comprises preferably 1 to 4 carbon atoms and is preferably unsubstituted or substituted by halogen and more preferred unsubstituted. According to compounds of formula II, alkyl is preferably selected from the group consisting of methyl, ethyl, trifluoro methyl, pentafluoro ethyl, isopropyl, n-butyl, tert.-butyl, 2-amino ethyl, N-methyl-2-amino ethyl, N,N-dimethyl-2-amino ethyl, N-ethyl-2-amino ethyl, N,N-diethyl-2-amino ethyl, 2-hydroxy ethyl, 2-methoxy ethyl and 2-ethoxy ethyl, further preferred of the group consisting of 2-butyl, n-pentyl, neo-nentyl, isopentyl, hexyl and n-decyl, more preferred of methyl, ethyl, trifluoro methyl, isopropyl and tert.-butyl.

In compounds of formula II, alkenyl is preferably selected from the group consisting of allyl, 2- or 3-butenyl, isobutenyl, sec-butenyl, furthermore preferably 4-pentenyl, isopentenyl and 5-hexenyl.

In compounds of formula II, alkylene is preferably unbranched and is more preferably methylene or ethylene, furthermore preferably propylene or butylene.

In compounds of formula II, alkylenecycloalkyl preferably has 5 to 10 carbon atoms and is preferably methylenecyclopropyl, methylenencyclobutyl, furthermore preferably methylenecyclopentyl, methylenecyclohexyl or methylenecycloheptyl, furthermore alternatively ethylenecyclopropyl, ethylenecyclobutyl, ethylenecyclopentyl, ethylenecyclohexyl or ethylenencycloheptyl, propylenecyclopentyl, propylenecyclohexyl, butylenecyclopentyl or butylenecyclohexyl.

In compounds of formula II, the term “alkoxy” preferably comprises groups of formula O-alkyl, where alkyl is an alkyl group as defined above. More preferred, alkoxy is selected from group consisting of methoxy, ethoxy, n-propoxy, isopropoxy, n-butoxy, tert.-butoxy and halogenated, especially perhalogenated, derivatives thereof. Preferred perhalogenated derivatives are selected from the group consisting of O—CCl₃, O—CF₃, O—C₂Cl₅, O—C₂F₅, O—C(CCl₃)₃ and O—C(CF₃)₃.

In compounds of formula II, the term “alkoxyalkyl” preferably comprises branched and unbranched residues, more preferred unbranched residues, of formula CᵤH₂ᵤ₊₁—O—(CH₂)ᵥ, wherein u and v are independently from each other 1 to 6. Especially preferred is u=1 and v 1 to 4.

In compounds of formula II the term “alkoxyalkyl” includes alkoxyalkyl groups as defined above, wherein one or more of the hydrogen atoms are substituted by halogen, for example up to perhalo alkoxyalkyl.

In compounds of formula II, cycloalkyl preferably has 3-7 carbon atoms and is preferably cyclopropyl or cyclobutyl, furthermore preferably cyclopentyl or cyclohexyl, furthermore also cycloheptyl, particularly preferably cyclopentyl. The term “cycloalkyl”, as used herein preferably also includes saturated heterocyclic groups, wherein one or two carbon atoms are substituted by hetero atoms, selected from the group consisting of O, NH, NA and S, wherein A is as defined as above/below.

In compounds of formula II, Ar³ to Ar⁶ are preferably selected independently from one another from phenyl, naphthyl and biphenyl which is optionally substituted by one or more substituents, selected from the group consisting of A, Hal, NO₂, CN, OR¹⁵, NR¹⁵R¹⁶, COOR⁵, CONR¹⁵R¹⁶, NR¹⁵COR¹⁶, NR¹⁵CONR¹⁵R¹⁶, NR¹⁶SO₂A, COR¹⁵, SO₂R¹⁵R¹⁶, S(O)ₙA and OOCR¹⁵.

In compounds of formula II, Het is preferably an optionally substituted aromatic heterocyclic residue and even more preferred and optionally substituted saturated heterocyclic residue, wherein the substituents are preferably selected from A, CN and Hal. Even more preferred, Het is selected from the group consisting of 1-piperidyl, 1-piperazyl, 1-(4-methyl)-piperazyl, 4-methylpiperazin-1-yl amine, 4-morpholinyl, 1-pyrrolidinyl, 2-pyrrolidinyl, 3-pyrrolidinyl, 1-pyrazolidinyl 1-(2-methyl)pyrazolidinyl, 1-imidazolidinyl or 1-(3-methyl)-imidazolidinyl, thiophen-2-yl, thiophen-3-yl, 2-pyridyl, 3-pyridyl, 4-pyridyl, 2-oxazolyl, 4-oxazolyl, 5-oxazolyl, 2-thiazolyl, 4-thiazolyl, 5-thiazolyl, chinolinyl, isochinolinyl, 2-pyridazyl, 4-pyridazyl, 2-pyrimidyl, 4-pyrimidyl, 5-pyrimidyl, 2-pyrazinyl and 3-pyrazinyl. Especially the thiophenyl and the pyridyl residues can optionally be substituted by one or more cyano groups.

In compounds of formula II, saturated heterocyclyl is preferably a substituted or unsubstituted saturated heterocyclic residue, more preferred an unsubstituted saturated heterocyclic residue, preferably selected from the saturated groups given above in the definition of Het.

In compounds of formula II, aromatic hydrocarbons containing 6 to 14 carbon atoms and ethylenical unsaturated or aromatic heterocyclic residues containing 3 to 10 carbon atoms and one or two heteroatoms, independently selected from N, O and S, are preferably selected from the definitions given herein for aryl, heteroaryl and/or Het. Heteroaryl is more preferably furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, benzofuranyl, benzothiophenyl, indolyl, indazolyl and even more preferably pyridinyl, pyrimidyl, chinolinyl, isochinolinyl, thiophenyl, thiadiazolyl, benzothiadiazolyl, benzofuranyl, benzothiophenyl, indolyl, indazolyl, benzimidazolyl, benzopyrazolyl, benzoxazolyl, benzisoxazolyl, benzothiazolyl, benzisothiazolyl, benzoxadiazolyl, benzooxazinyl, benzodioxolyl, benzodioxanyl, benzoxadiazolyl, benzotriazolyl, benzooxazinyl, dihydrobenzofuranyl, dihydrobenzoisofuranyl, chromanyl, isochromanyl, benzodioxinyl, benzodioxolyl, benzothiadiazolyl, oxazolyl, isoxazolyl, pyrazolyl and/or imidazolyl. Aryl more preferably refers to an optionally substituted benzene ring or to an optionally substituted benzene ring system fused to one or more optionally substituted benzene rings to form, for example, anthracene, phenanthrene, or napthalene ring systems. Even more preferably, aryl is selected from the group consisting of phenyl, 2-naphthyl, 1-naphthyl, biphenyl.

In compounds of formula II, Ar¹ is preferably selected from the group consisting of phenyl, pyridinyl, pyrimidyl, chinolinyl, isochinolinyl, thiophenyl, thiadiazolyl, indolyl, benzothiadiazolyl, benzotriazolyl, benzodioxolyl, oxazolyl, isoxazolyl, pyrazolyl and imidazolyl, and especially from phenyl, indolyl, benzotriazolyl and benzodioxolyl.

In compounds of formula II, Ar² is preferably selected from the group consisting of phenyl, pyridinyl, pyrrazolyl, pyrimidyl, chinolinyl, isochinolinyl, thiophenyl, thiadiazolyl, benzothiadiazolyl, oxazolyl, isoxazolyl, pyrazolyl and imidazolyl, and especially preferred from phenyl, pyridinyl and pyrrazolyl.

Preferably, the sum of h and l exceeds 0.

A preferred aspect of the instant invention relates to compounds of formula II, wherein n is 0 or 1 and especially 0.

Another preferred aspect of the instant invention relates to compounds of formula II, wherein n is 0 in the residues R⁸, R⁹ and/or R¹⁰ and especially in R¹⁰.

Another preferred aspect of the instant invention relates to compounds of formula II, wherein X represents a bridging group, selected from (CR¹¹R¹²)ₕ or (CHR¹¹)ₕ-Q-(CHR¹²)ᵢ.

Another preferred embodiment of the instant invention relates to compounds of formula II wherein q is 1, i.e. the phenyl group bound to the methylene group of the diacylhydrazine moiety is substituted by one substituent, preferably a substituent as defined above and more preferably a substituent selected from alkyl and hal, and especially selected from CH₃, CH₂CH₃ and Hal.

The invention relates in particular to compounds of the formula II in which at least one of said radicals has one of the preferred meanings given above.

Some more preferred groups of compounds may be expressed by the following sub-formulae II.1) to II.11), which correspond to the formula II and in which radicals not denoted in greater detail are as defined in the formula II, but in which
II.1) Ar¹ is phenyl,
   R⁸ is H, methyl, ethyl, n-propyl, isopropyl, n-butyl, 2-butyl, tert.-butyl, methoxy, ethoxy, n-propoxy, isopropoxy, n-butoxy, 2-butoxy, tert.-butoxy, Hal, CHal₃ or OCHal₃ and
   p is 1 or 2;
II.2) E, G, M,
   Q, U are carbon atoms and
   R⁹ is H;
II.3) A is phenyl, pyridinyl or pyrrolyl,
   R¹⁰ is H or CONCH₃,
   r is 1 and
   X is O or a bond;
II.4) Ar¹ is phenyl,
   R⁸ is H, methyl, ethyl, n-propyl, isopropyl, n-butyl, 2-butyl, tert.-butyl, methoxy, ethoxy, n-propoxy, isopropoxy, n-butoxy, 2-butoxy, tert.-butoxy, Hal, CHal₃ or OCHal₃,
   p is 1 or 2,
   E, G, M,
   Q, U are carbon atoms and
   R⁹ is H,
II.5) Ar¹ is phenyl,
   R⁸ is H, methyl, ethyl, n-propyl, isopropyl, n-butyl, 2-butyl, tert.-butyl, methoxy, ethoxy, n-propoxy, isopropoxy, n-butoxy, 2-butoxy, tert.-butoxy, Hal, CHal₃ or OCHal₃,
   p is 1 or 2,
   Ar² is phenyl, pyridinyl or pyrrolyl,
   R¹⁰ is H or CONCH₃,
   r is 1 and
   X is O or a bond;
II.6) E, G, M,
   Q, U are carbon atoms,
   R⁹ is H,
   Ar² is phenyl, pyridinyl or pyrrolyl,
   R¹⁰ is H or CONCH₃,
   r is 1 and
   X is O or a bond;
II.7) Ar¹ is phenyl,
   R⁸ is H, methyl, ethyl, n-propyl, isopropyl, n-butyl, 2-butyl, tert.-butyl, methoxy, ethoxy, n-propoxy, isopropoxy, n-butoxy, 2-butoxy, tert.-butoxy, Hal, CHal₃ or OCHal₃,
   p is 1 or 2,
   E, G, M,
   Q, U are carbon atoms,
   R⁹ is H,
   Ar² is phenyl, pyridinyl or pyrrolyl,
   R¹⁰ is H or CONCH₃,
   r is 1 and
   X is O or a bond;
II.8) Ar¹ is phenyl,
   R⁸ is H, methyl, ethyl, n-propyl, isopropyl, n-butyl, 2-butyl, tert.-butyl, methoxy, ethoxy, n-propoxy, isopropoxy, n-butoxy, 2-butoxy, tert.-butoxy, Hal, CHal₃ or OCHal₃,
   p is 1 or 2,
   E, G, M, Q, U are carbon atoms,
   R⁹ is H.
   Ar² is phenyl, pyridinyl or pyrrolyl,
   R¹⁰ is H or CONCH₃,
   r is 1 and
   X is O;
II.9) Ar¹ is phenyl,
   R⁸ is H, methyl, ethyl, n-propyl, isopropyl, n-butyl, 2-butyl, tert.-butyl, methoxy, ethoxy, n-propoxy, isopropoxy, n-butoxy, 2-butoxy, tert.-butoxy, Hal, CHal₃ or OCHal₃,
   p is 1 or 2,
   E, G, M,
   Q, U are carbon atoms,
   R⁹ is H,
   Ar² is phenyl, pyridinyl or pyrrolyl,
   R¹⁰ is H or CONCH₃,
   r is 1 and
   X is a bond;
II.10) Ar¹ is phenyl,
   R⁸ is H, methyl, ethyl, n-propyl, isopropyl, n-butyl, 2-butyl, tert.-butyl, methoxy, ethoxy, n-propoxy, isopropoxy, n-butoxy, 2-butoxy, tert.-butoxy, Hal, CHal₃ or OCHal₃,
   p is 1 or 2,
   E, G, M, Q, U are carbon atoms,
   R⁹ is H,
   Ar² is phenyl or pyridinyl,
   R¹⁰ is H or CONCH₃, where Ar² is pyridinyl, R¹⁰ is preferably bonded in a vicinal position to the nitrogen atom of the pyrindiyl residue,
   r is 1 and
   X is O;
II.11) Ar¹ is phenyl,
   R⁸ is H, methyl, ethyl, n-propyl, isopropyl, n-butyl, 2-butyl, tert.-butyl, methoxy, ethoxy, n-propoxy, isopropoxy, n-butoxy, 2-butoxy, tert.-butoxy, Hal, CHal₃ or OCHal₃,
   p is 1 or 2,
   E, G, M, Q, U are carbon atoms,
   R⁹ is H,
   Ar² is phenyl, pyridinyl or pyrrolyl,
   R¹⁰ is H and
   X is a bond;

Another more preferred embodiment of the instant invention relates to compounds of formula II and preferably one or more of sub formulae II.1) to II.11), wherein Y is selected from the group consisting of O, S and NR²¹.

Another even more preferred embodiment of the instant invention relates to compounds of formula II and preferably one or more of sub formulae II.1) to II.11), wherein Y is selected from the group consisting of O and S.

Another even more preferred embodiment of the instant invention relates to compounds of formula II and preferably one or more of sub formulae II.1) to II.20), wherein Y is O.

Another preferred embodiment of the instant invention relates to compounds of formula II and preferably one or more of sub formulae II.1) to II.20), wherein Ar² is pyridinyl.

Where Ar² is pyrrolyl, said residue is preferably bonded to X via the nitrogen atom.

Another preferred embodiment of the instant invention relates to compounds of formula II and preferably one or more of formulae II.1) to II.11), wherein (R⁸)ₚ—Ar¹ is selected from the group consisting of 3-acetyl-phenyl, 4-acetyl-phenyl, 2-bromo-phenyl, 3-bromo-phenyl, 4-bromo-phenyl, 4-bromo-2-chloro-phenyl, 4-bromo-3-methyl-phenyl, 4-bromo-3-trifluoromethyl-phenyl, 2-chloro-phenyl, 2-chloro-4-trifluoromethyl-phenyl, 2-chloro-5-trifluoromethyl-phenyl, 3-chloro-phenyl, 3-chloro-4-methyl-phenyl, 3-chloro-4-methoxy-phenyl, 3-chloro-4-methoxy-phenyl, 4-chloro-phenyl, 4-chloro-2-trifluoromethyl-phenyl, 4-chloro-3-trifluoromethyl-phenyl, 4-chloro-2-methyl-phenyl, 5-chloro-2-methyl-phenyl, 5-chloro-2-methoxy-phenyl, 2,3-dichloro-phenyl, 2,4-dichloro-phenyl, 2,5-dichloro-phenyl, 3,4-dichloro-phenyl, 3,5-dichloro-phenyl, 2,4,5-trichloro-phenyl, 4-fluoro-phenyl, 4-fluoro-3-trifluoromethyl-phenyl, 4-ethoxy-phenyl, 2-methoxy-phenyl, 2-methoxy-5-trifluoromethyl-phenyl, 4-methoxy-phenyl, 2,5-dimethoxy-phenyl, 2-trifluoromethyl-phenyl, 3-trifluoromethyl-phenyl, 3-trifluoromethoxy-phenyl, 4-trifluoromethyl-phenyl, 4-trifluoromethoxy-phenyl, 3,5-bis-trifluoromethyl-phenyl, 3-methoxy-phenyl, 3-methylsulfanyl-phenyl, 4-methylsulfanyl-phenyl, O-tolyl (2-methyl-phenyl), m-tolyl (3-methyl-phenyl), p-tolyl (4-methyl-phenyl), 2,3-dimethyl-phenyl, 2,3-dimethyl-phenyl, 2,5-dimethyl-phenyl, 3,4-dimethyl-phenyl, 3,5-dimethyl-phenyl, 2-ethyl-phenyl, 3-ethyl-phenyl, 4-ethyl-phenyl, 4-isopropyl-phenyl, 4-n-butyl-phenyl, 4-tert-butyl-phenyl, 4-n-butoxy-phenyl and 4-tert.-butoxy-phenyl.

Another preferred embodiment of the instant invention relates to compounds of formula II and preferably one or more of sub formulae II.1) to II.11), wherein X is bonded in the para- (p-) or meta- (m-)position to the phenyl residue that is bonded directly to the diacylhydrazine moiety.

Another preferred embodiment of the instant invention relates to compounds of formula II and preferably one or more of sub formulae II.1) to II.11), wherein Ar² is a pyridinyl residue and wherein said pyridinyl residue is bonded to X in the 3- or 4-position, preferably the 4-position, relative to the nitrogen atom of the pyridinyl residue.

Another preferred embodiment of the instant invention relates to compounds of formula II and preferably one or more of sub formulae II.1) to II.11), wherein Ar² comprises one or more substituents R¹⁰ and wherein one or two, preferably one substituent R¹⁰ is selected from unsubstituted or substituted carbamoyl moieties. Substituted carbamoyl moieties are preferably selected from CONHR²³ or CONR²³R²⁴, preferably CONHR²³, wherein R²³ and R²⁴ are independently selected from the definitions given for R⁸, more preferably selected from alkyl, preferably methyl, ethyl, propyl and butyl, (CH₂)ₙNR¹¹R¹² and (CH₂)ₙOR¹², wherein R¹¹, R¹² and n are as defined above. In this embodiment, n is preferably not 0 and more preferred 1 to 3 and especially 1 or 2. Preferred examples for R²³ are selected from the group consisting of methyl, ethyl, CH₂CH₂NH₂, CH₂CH₂N(CH₃)₂, CH₂CH₂N(CH₂CH₃)₂, CH₂CH₂OH, CH₂CH₂OCH₃ and CH₂CH₂OCH₂CH₃.

Another preferred embodiment of the instant invention relates to compounds of formula II and preferably one or more of sub formulae II.1) to II.11), wherein Ar² comprises one or more substituents R¹⁰ and wherein one or two, preferably one substituent R¹⁰ is selected from substituted carbamoyl moieties. Substituted carbamoyl moieties are preferably selected from CONHR²³, wherein R²³ is preferably unsubstituted C₁-C₄-alkyl and especially methyl.

Another especially preferred embodiment of the instant invention relates to compounds of formula II and preferably one or more of sub formulae II.1) to II.11), wherein one or more features of the above and below mentioned embodiments are combined in one compound.

Subject of the present invention are therefore preferably compounds of formula II according to any one of the formulae formula IIa, IIb, IIc, IId, IIe, IIf, IIg, IIh, IIi, IIj, IIk, IIl, IIm, IIn, IIo, IIp, IIq, IIr, IIu, IIv, IIw and IIx 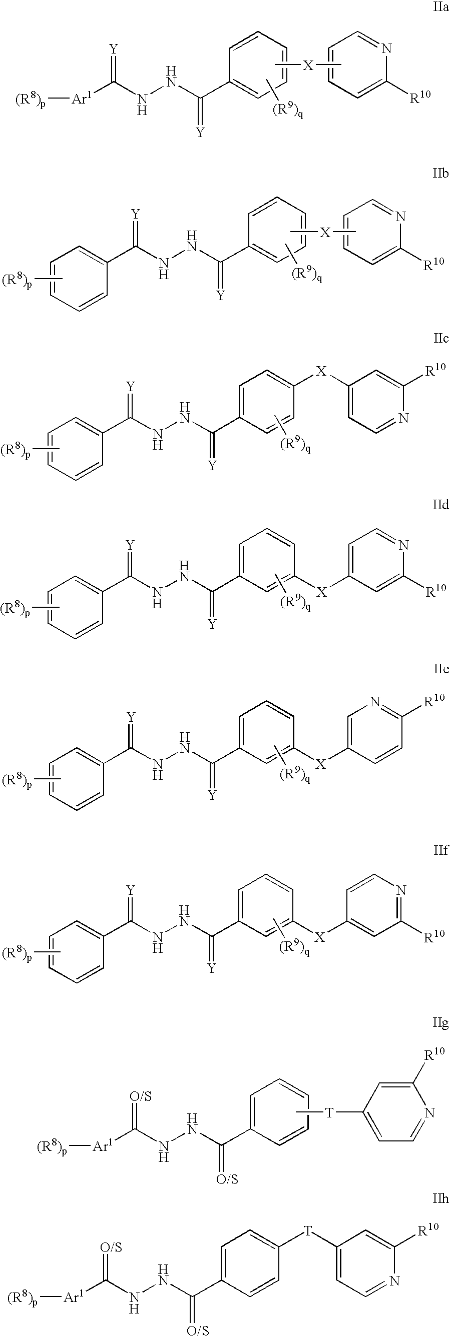 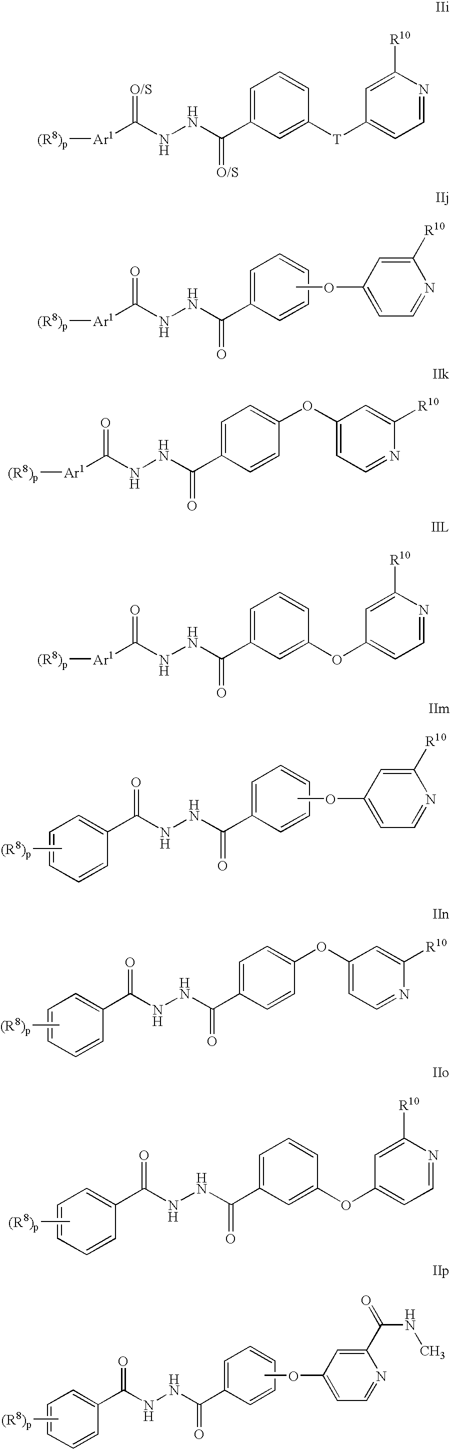 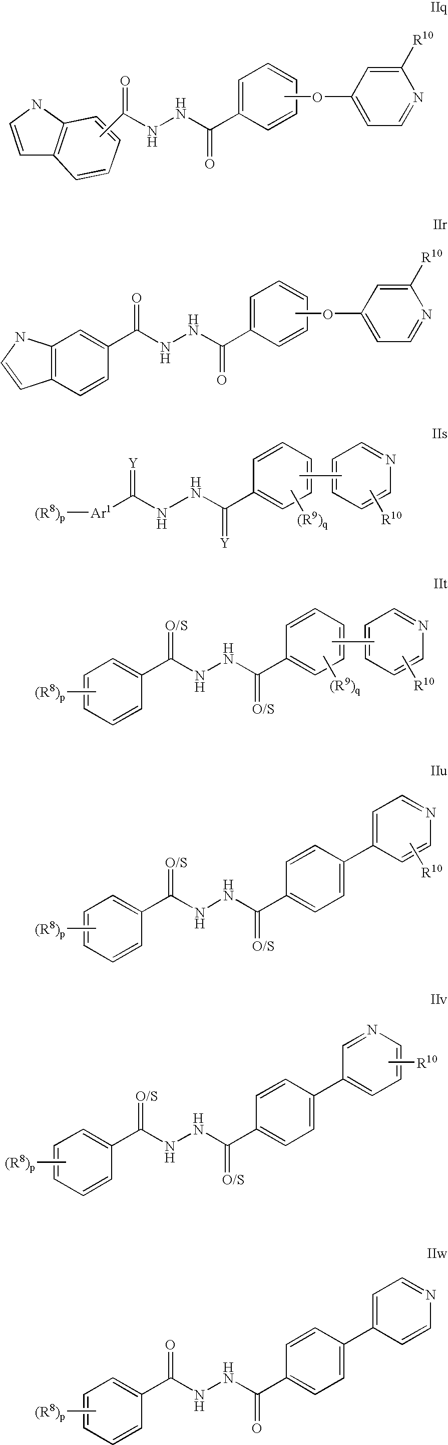 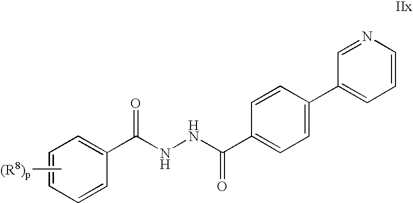
wherein Ar¹, R⁸, p, Y, X, R⁹, q, R¹⁰ and r are as defined above and below, and preferably as defined in sub formulae II.1) to II.11) and/or the embodiments related thereto.

Another preferred embodiment of the instant invention relates to compounds of formula II wherein
E, G, M, U and Q are carbon atoms,
X is O or a bond,
Y is O,
Ar¹ is phenyl or indolyl,
Ar² is pyridinyl,
R⁸ is H, methyl, ethyl, n-propyl, isopropyl, n-butyl, 2-butyl, tert.-butyl, methoxy, ethoxy, n-propoxy, isopropoxy, n-butoxy, 2-butoxy, tert.-butoxy, Hal, CHal₃ or OCHal₃,
R¹⁰ is H or CONCH₃,
p is 0, 1, 2 or 3,
q is O,
r is 1 and
   where X is O, R¹⁰ is especially preferred CONCH₃ and
   where X is a bond, R¹⁰ is especially preferred H.

Yet another preferred embodiment relates to compounds of formula II wherein

E, G, M, U and Q are carbon atoms,

X is O, S or NR¹⁵ and

Y is O.

It is understood that when a residue, for example R⁸, R⁹, R¹⁰ or R¹⁴ or R²³, is comprised two or more times in one or more of the formulae I, II and the sub formulae corresponding thereto, it is in each case independently from one another selected from the meanings given for the respective residue. For example, R¹¹ and R¹² are defined to be independently selected from a group consisting of H, A, (CH₂)ₘAr³ and (CH₂)ₘHet. Then (CH₂)ₙNR¹¹(CH₂)ₘNR¹²R¹² can be (CH₂)ₙNA(CH₂)ₘNA₂ (if R¹¹=A, R¹²=A and R¹²=H) as well as (CH₂)ₙNA(CH₂)ₘNHA (if R¹¹=A, R¹²=H and R²=A or (CH₂)ₙNA(CH₂)ₘNH(CH₂ₘHet (if R¹¹=A, R¹²=H and R¹²=(CH₂)ₘHet). Accordingly, if a compound of formula II comprises one residue R⁸, R⁹ and R¹⁰, then for example R⁸, R⁹ and R¹⁰ can all be (CH₂)ₙCOOR¹³, wherein all residues R¹³ are the same (for example CH₂Hal, wherein Hal is Cl; then all residues R⁸, R⁹ and R¹⁰ are the same) or different (for example CH₂Hal, wherein in R⁸ Hal is Cl; in R⁹ Hal is F; and in R¹⁰ Hal is Br; then all residues R⁸, R⁹ and R¹⁰ are different); or for example R⁸ is (CH₂)ₙCOOR¹³, R⁹ is NO₂ and R¹⁰ is (CH₂)ₙSR¹¹, wherein R¹¹ and R¹³ can be the same (for example both can be H or both can be A which is methyl) of different (for example R¹¹ can be H and R¹³ can be A which is methyl).

If not stated otherwise, reference to compounds of formula I and formula II also includes the sub formulae related thereto, especially sub formulae II.1) to II.11) and IIa to IIx.

Subject of the instant invention are especially those compounds of formula I and/or formula II, in which at least one of the residues mentioned in said formulae has one of the preferred or especially preferred meanings given above and below.

The present invention further relates to compounds (1) to (224) of formula A-CO—NH—NH—CO—B, wherein A and B are as given in the table below:

| | A | B |
|---|---|---|
| (1) | 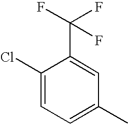 | 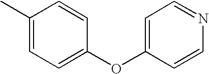 |
| (2) | 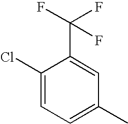 | 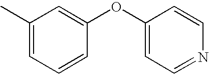 |
| (3) | 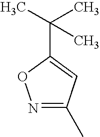 | 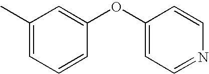 |
| (4) | 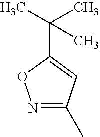 | 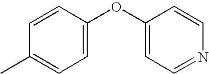 |
| (5) | 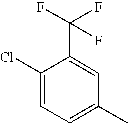 | 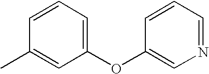 |
| (6) | 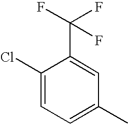 | 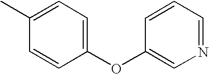 |
| (7) | 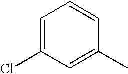 | 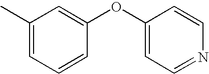 |
| (8) | 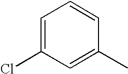 | 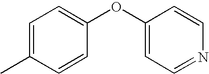 |
| (9) | 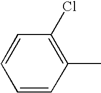 | 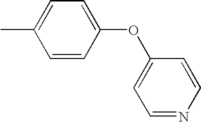 |
| (10) | 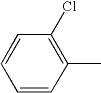 | 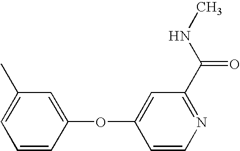 |
| (11) | 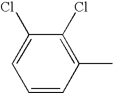 | 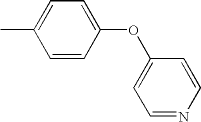 |
| (12) | 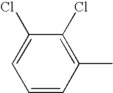 | 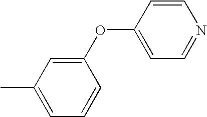 |
| (13) | 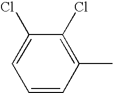 | 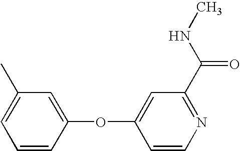 |
| (14) | 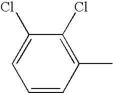 | 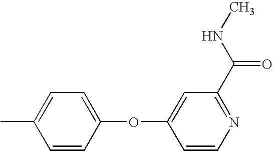 |
| (15) | 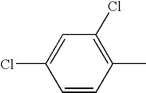 | 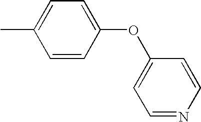 |
| (16) | 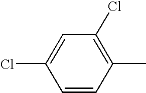 | 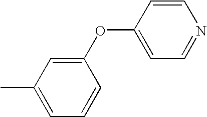 |
| (17) | 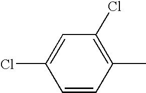 | 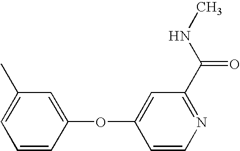 |
| (18) | 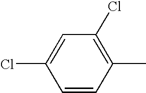 | 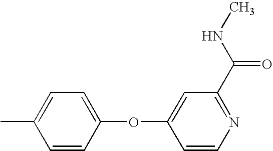 |
| (19) | 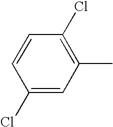 | 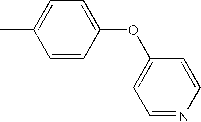 |
| (20) | 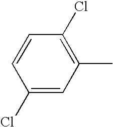 | 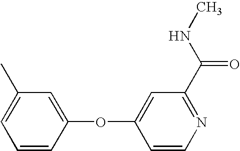 |
| (21) | 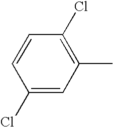 | 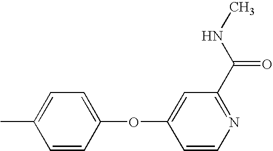 |
| (22) | 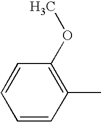 | 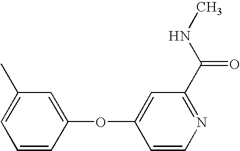 |
| (23) | 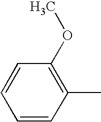 | 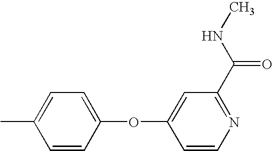 |
| (24) | 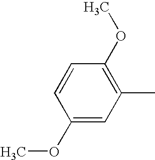 | 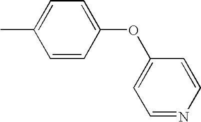 |
| (25) | 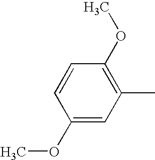 | 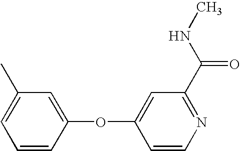 |
| (26) | 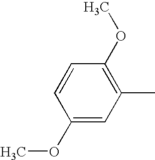 | 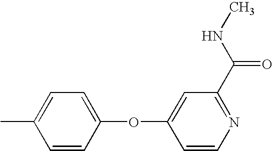 |
| (27) | 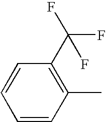 | 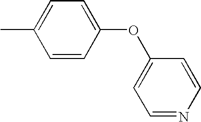 |
| (28) | 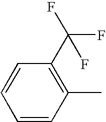 | 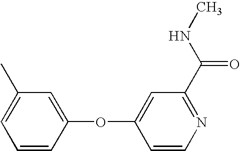 |
| (29) | 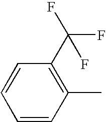 | 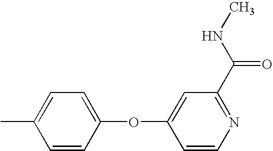 |
| (30) | 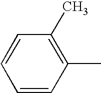 | 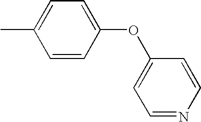 |
| (31) | 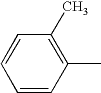 | 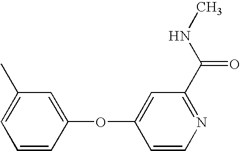 |
| (32) | 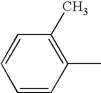 | 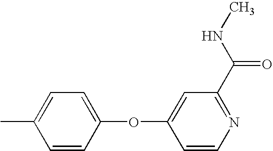 |
| (33) | 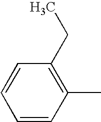 | 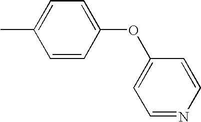 |
| (34) | 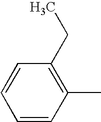 | 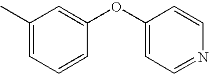 |
| (35) | 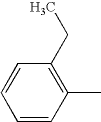 | 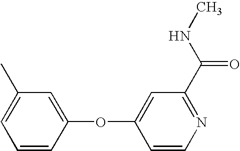 |
| (36) | 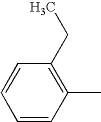 | 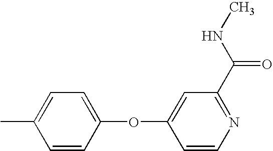 |
| (37) | 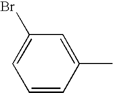 | 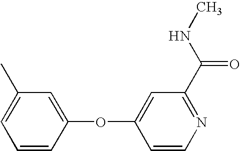 |
| (38) | 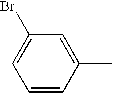 | 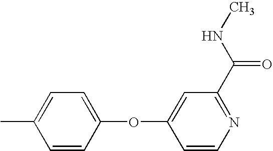 |
| (39) | 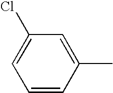 | 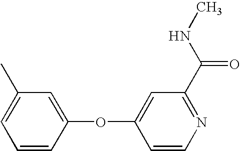 |
| (40) | 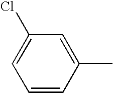 | 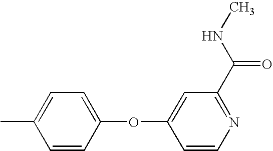 |
| (41) | 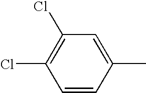 | 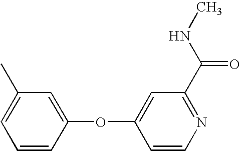 |
| (42) | 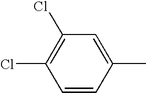 | 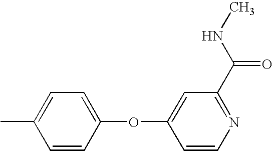 |
| (43) | 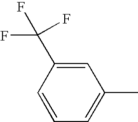 | 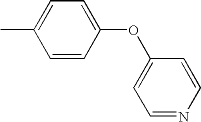 |
| (44) | 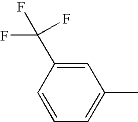 | 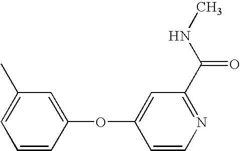 |
| (45) | 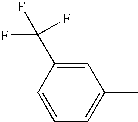 | 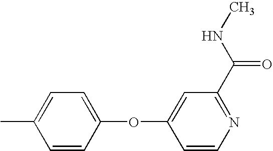 |
| (46) | 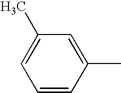 | 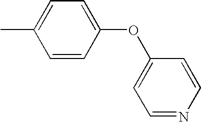 |
| (47) | 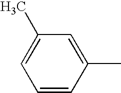 | 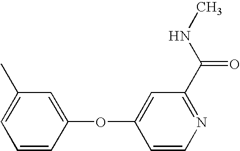 |
| (48) | 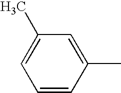 | 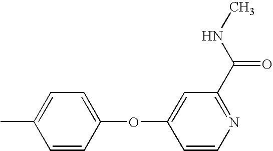 |
| (49) | 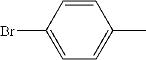 | 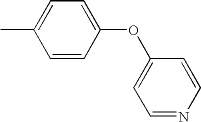 |
| (50) | 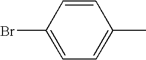 | 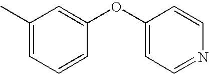 |
| (51) | 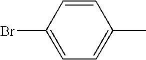 | 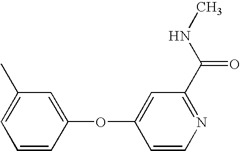 |
| (52) | 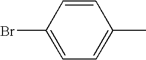 | 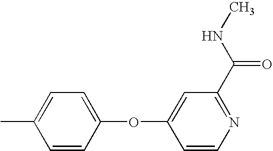 |
| (53) | 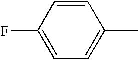 | 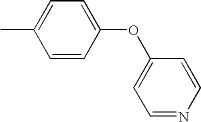 |
| (54) | 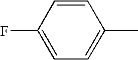 | 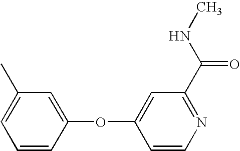 |
| (55) | 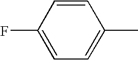 | 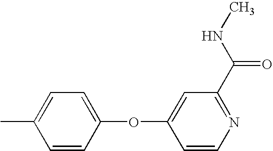 |
| (56) | 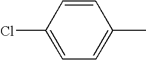 | 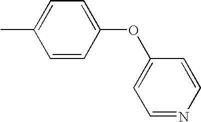 |
| (57) | 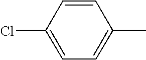 | 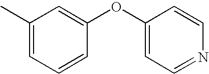 |
| (58) | 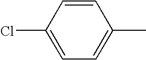 | 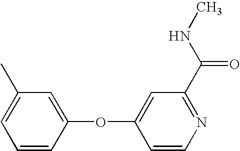 |
| (59) | 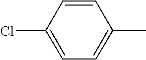 | 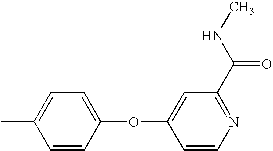 |
| (60) | 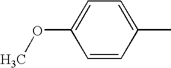 | 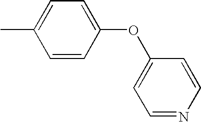 |
| (61) | 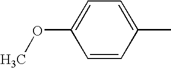 | 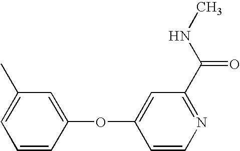 |
| (62) | 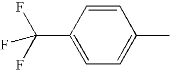 | 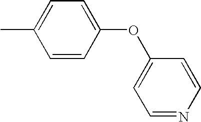 |
| (63) | 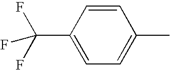 | 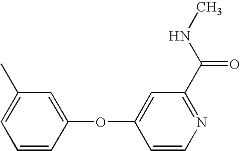 |
| (64) | 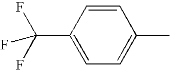 | 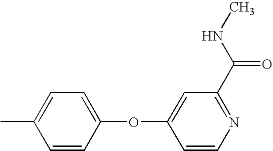 |
| (65) | 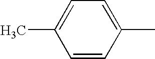 | 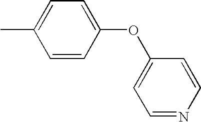 |
| (66) | 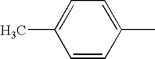 | 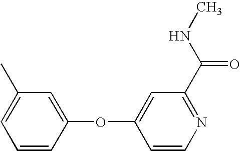 |
| (67) | 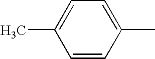 | 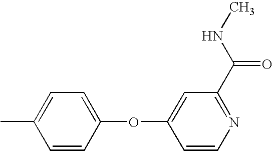 |
| (68) | 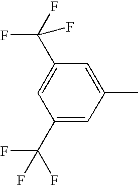 | 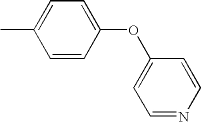 |
| (69) | 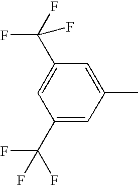 | 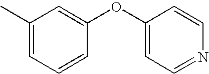 |
| (70) | 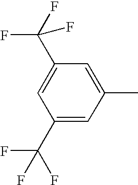 | 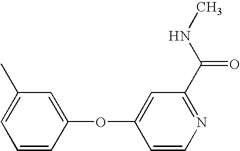 |
| (71) | 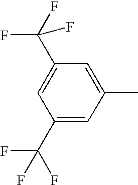 | 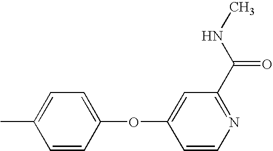 |
| (72) | 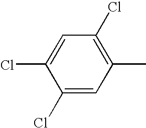 | 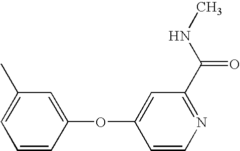 |
| (73) | 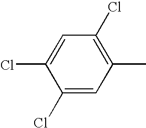 | 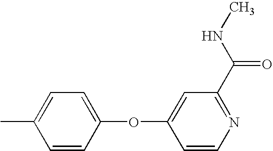 |
| (74) | 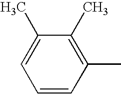 | 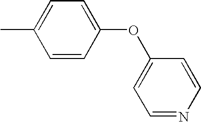 |
| (75) | 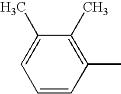 | 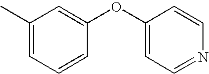 |
| (76) | 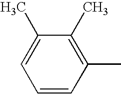 | 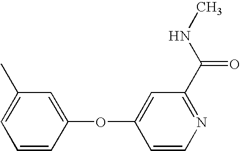 |
| (77) | 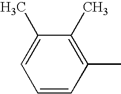 | 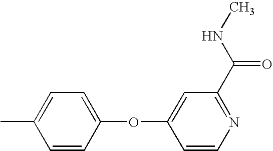 |
| (78) | 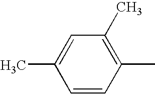 | 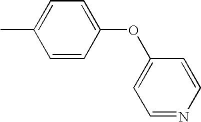 |
| (79) | 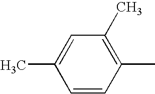 | 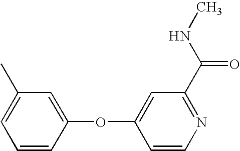 |
| (80) | 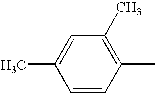 | 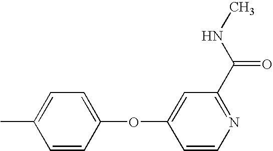 |
| (81) | 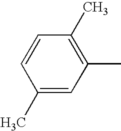 | 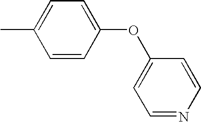 |
| (82) | 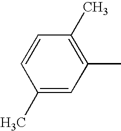 | 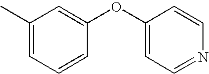 |
| (83) | 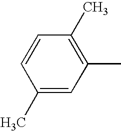 | 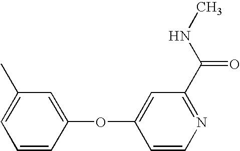 |
| (84) | 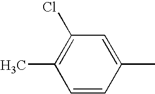 | 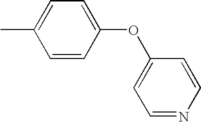 |
| (85) | 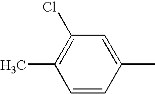 | 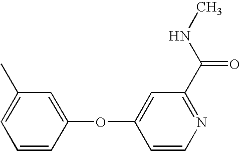 |
| (86) | 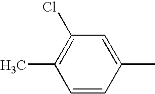 | 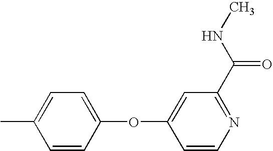 |
| (87) | 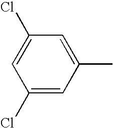 | 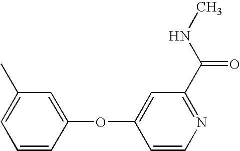 |
| (88) | 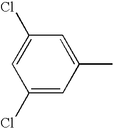 | 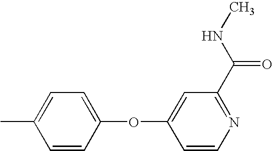 |
| (89) | 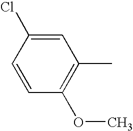 | 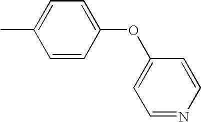 |
| (90) | 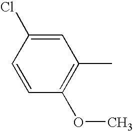 | 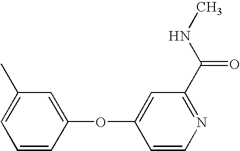 |
| (91) | 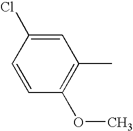 | 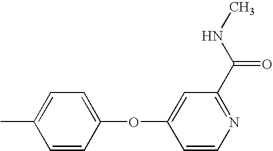 |
| (92) | 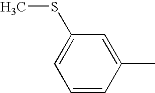 | 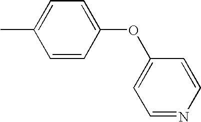 |
| (93) | 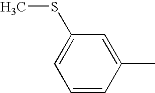 | 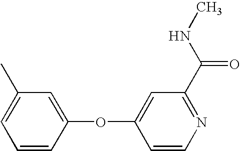 |
| (94) | 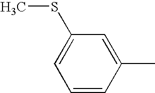 | 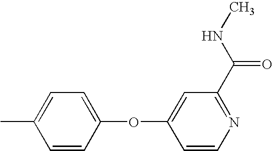 |
| (95) | 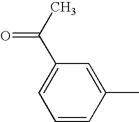 | 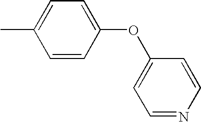 |
| (96) | 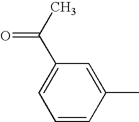 | 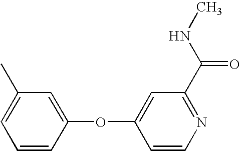 |
| (97) | 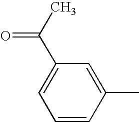 | 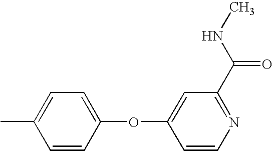 |
| (98) | 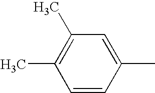 | 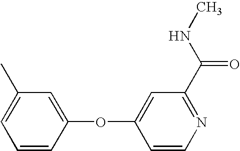 |
| (99) | 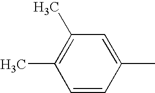 | 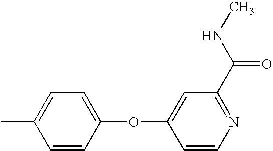 |
| (100) | 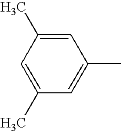 | 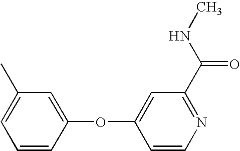 |
| (101) | 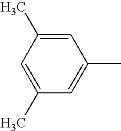 | 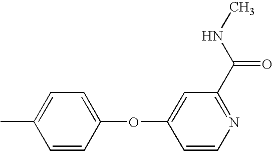 |
| (102) | 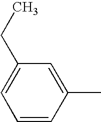 | 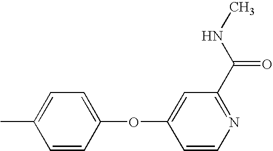 |
| (103) | 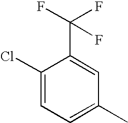 | 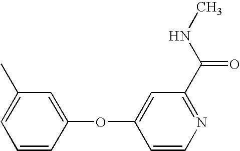 |
| (104) | 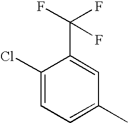 | 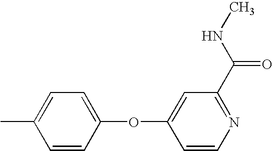 |
| (105) | 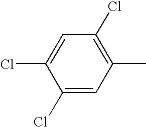 | 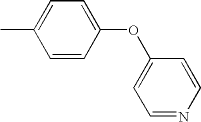 |
| (106) | 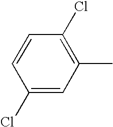 | 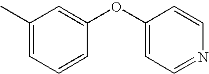 |
| (107) | 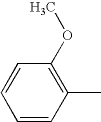 | 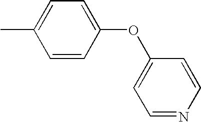 |
| (108) | 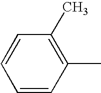 | 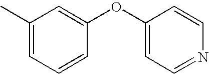 |
| (109) | 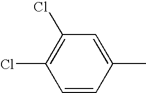 | 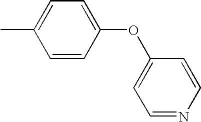 |
| (110) | 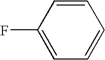 | 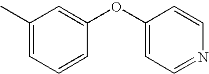 |
| (111) | 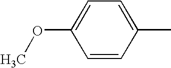 | 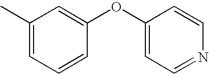 |
| (112) | 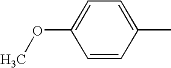 | 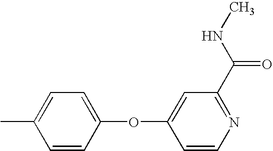 |
| (113) | 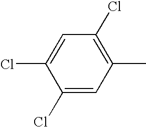 | 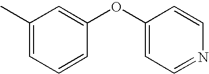 |
| (114) | 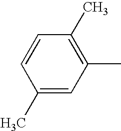 | 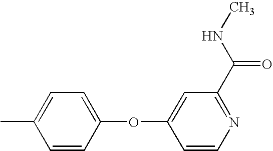 |
| (115) | 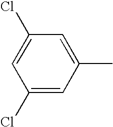 | 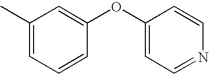 |
| (116) | 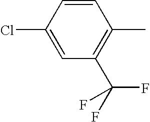 | 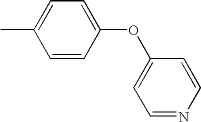 |
| (117) | 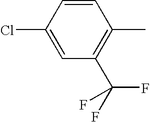 | 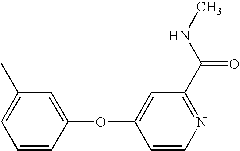 |
| (118) | 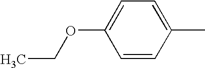 | 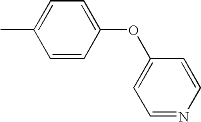 |
| (119) | 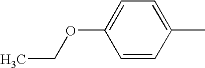 | 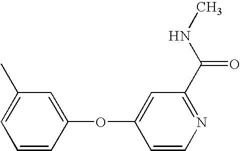 |
| (120) | 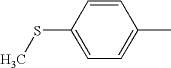 | 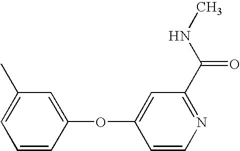 |
| (121) | 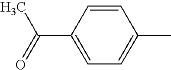 | 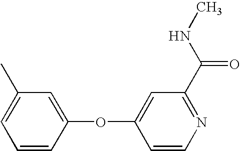 |
| (122) | 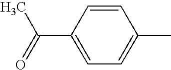 | 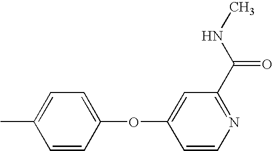 |
| (123) | 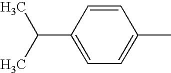 | 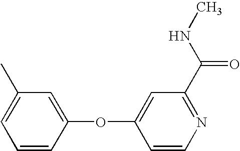 |
| (124) | 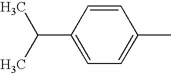 | 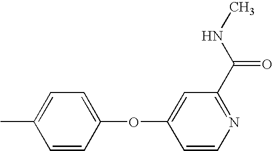 |
| (125) | 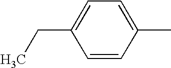 | 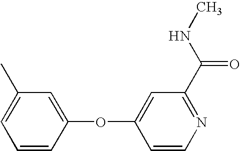 |
| (126) | 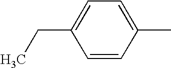 | 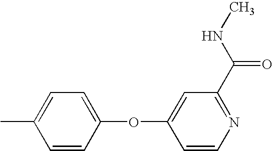 |
| (127) | 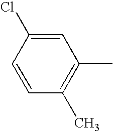 | 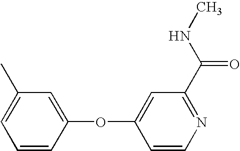 |
| (128) | 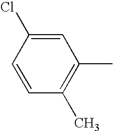 | 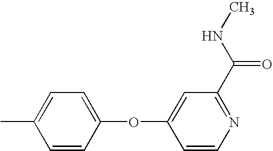 |
| (129) | 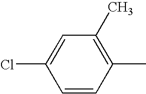 | 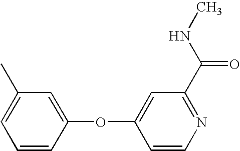 |
| (130) | 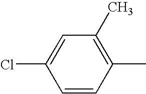 | 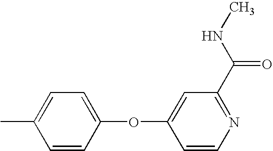 |
| (131) 2.62 | 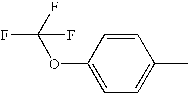 | 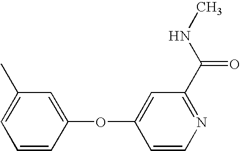 |
| (132) 2 | 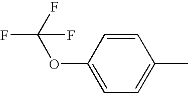 | 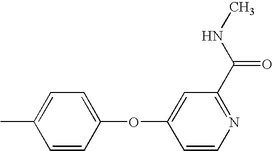 |
| (133) | 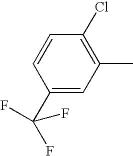 | 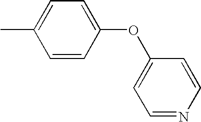 |
| (134) | 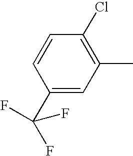 | 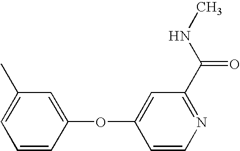 |
| (135) | 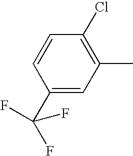 | 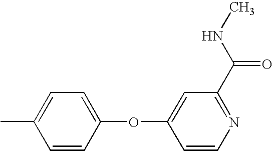 |
| (136) | 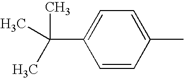 | 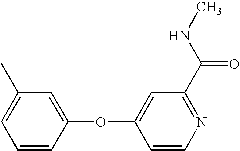 |
| (137) | 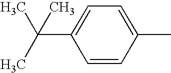 | 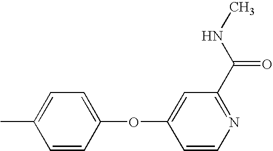 |
| (138) | 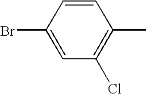 | 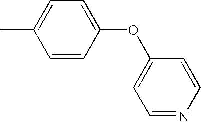 |
| (139) | 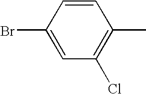 | 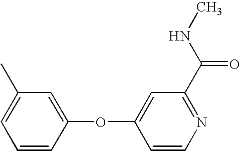 |
| (140) | 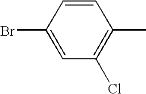 | 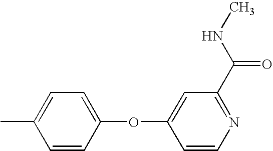 |
| (141) | 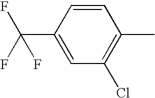 | 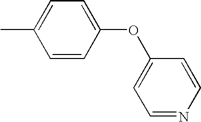 |
| (142) | 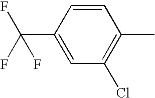 | 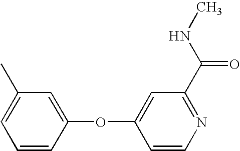 |
| (143) | 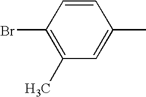 | 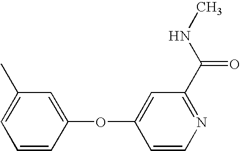 |
| (144) | 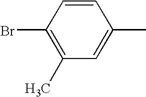 | 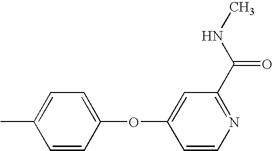 |
| (145) | 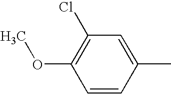 | 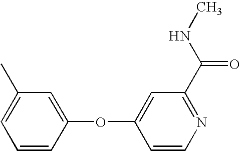 |
| (146) | 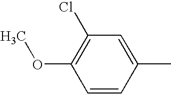 | 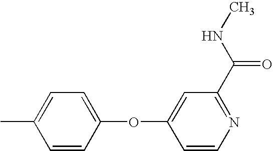 |
| (147) | 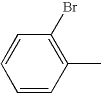 | 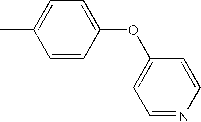 |
| (148) | 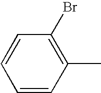 | 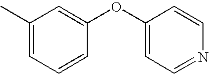 |
| (149) | 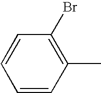 | 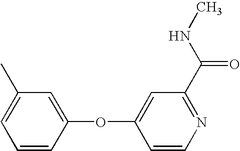 |
| (150) | 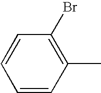 | 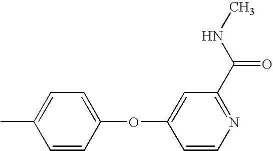 |
| (151) | 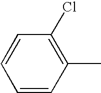 | 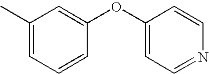 |
| (152) | 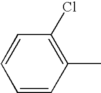 | 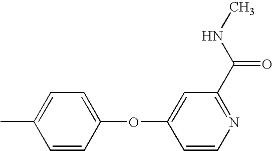 |
| (153) | 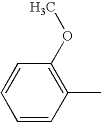 | 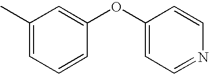 |
| (154) | 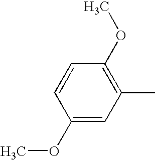 | 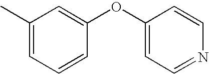 |
| (155) | 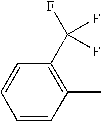 | 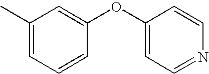 |
| (156) | 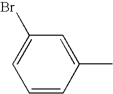 | 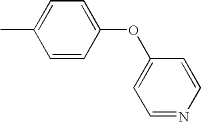 |
| (157) | 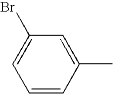 | 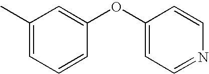 |
| (158) | 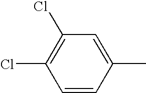 | 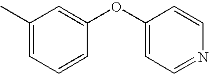 |
| (159) | 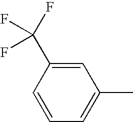 | 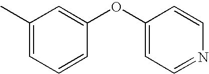 |
| (160) | 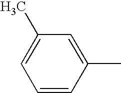 | 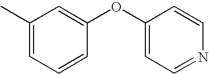 |
| (161) | 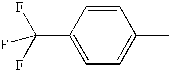 | 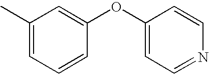 |
| (162) | 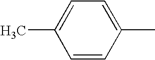 | 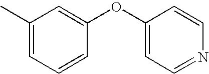 |
| (163) | 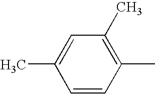 | 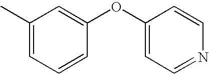 |
| (164) | 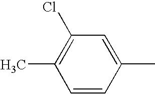 | 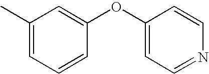 |
| (165) | 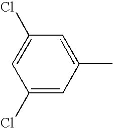 | 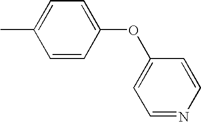 |
| (166) | 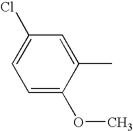 | 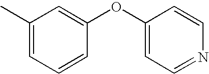 |
| (167) | 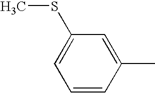 | 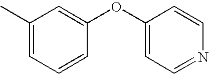 |
| (168) | 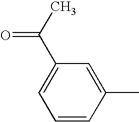 | 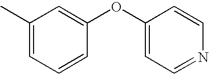 |
| (169) | 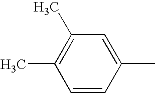 | 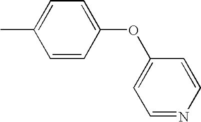 |
| (170) | 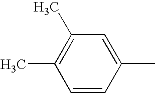 | 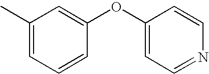 |
| (171) | 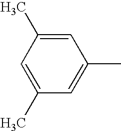 | 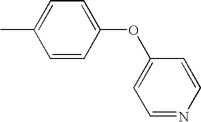 |
| (172) | 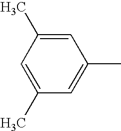 | 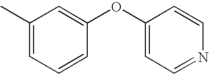 |
| (173) | 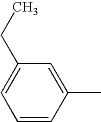 | 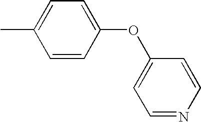 |
| (174) | 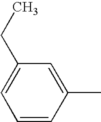 | 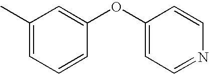 |
| (175) | 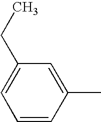 | 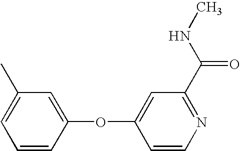 |
| (176) | 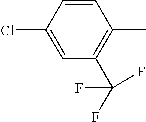 | 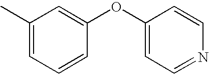 |
| (177) | 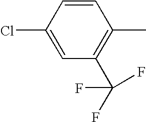 | 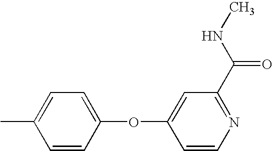 |
| (178) | 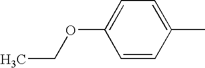 | 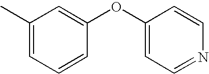 |
| (179) | 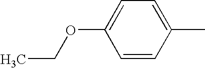 | 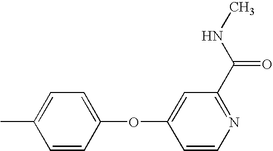 |
| (180) | 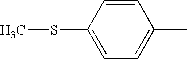 | 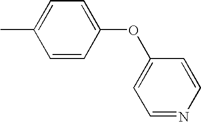 |
| (181) | 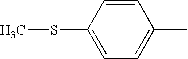 | 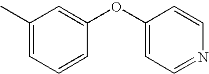 |
| (182) | 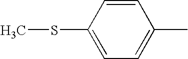 | 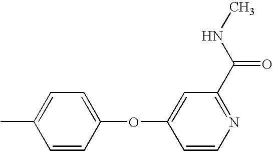 |
| (183) | 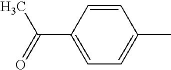 | 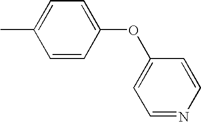 |
| (184) | 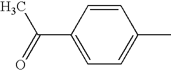 | 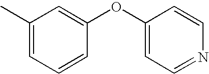 |
| (185) | 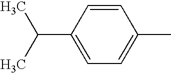 | 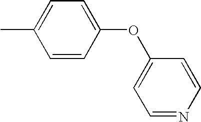 |
| (186) | 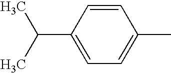 | 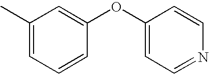 |
| (187) | 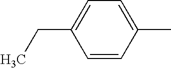 | 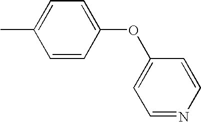 |
| (188) | 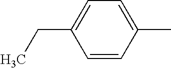 | 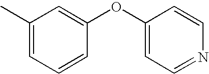 |
| (189) | 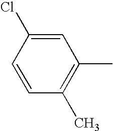 | 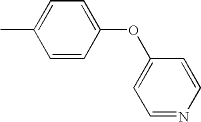 |
| (190) | 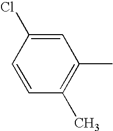 | 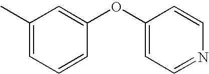 |
| (191) | 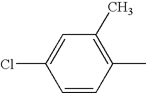 | 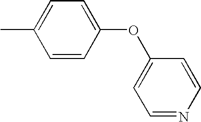 |
| (192) | 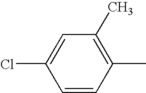 | 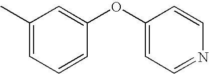 |
| (193) | 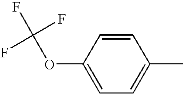 | 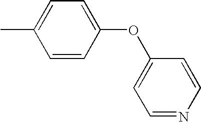 |
| (194) | 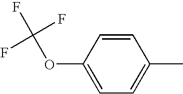 | 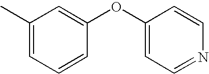 |
| (195) | 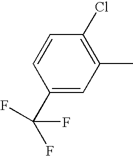 | 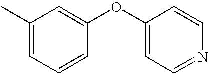 |
| (196) | 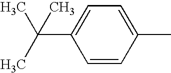 | 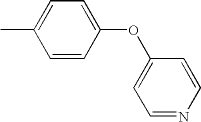 |
| (197) | 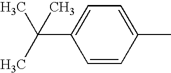 | 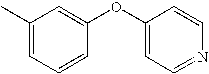 |
| (198) | 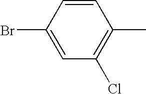 | 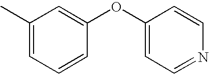 |
| (199) | 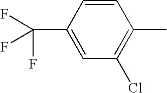 | 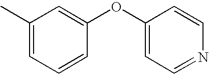 |
| (200) | 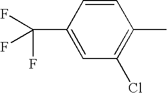 | 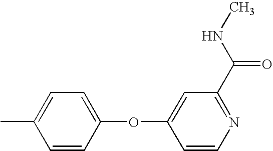 |
| (201) | 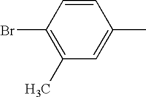 | 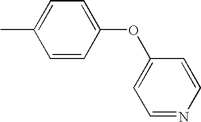 |
| (202) | 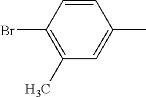 | 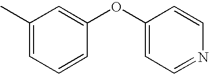 |
| (203) | 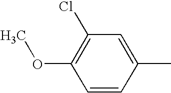 | 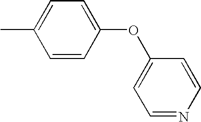 |
| (204) | 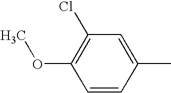 | 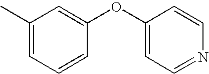 |
| (205) | 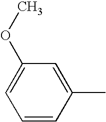 | 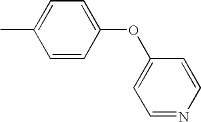 |
| (206) | 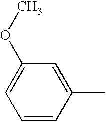 | 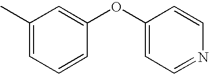 |
| (207) | 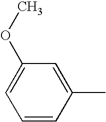 | 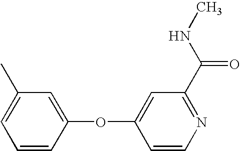 |
| (208) | 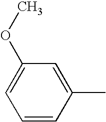 | 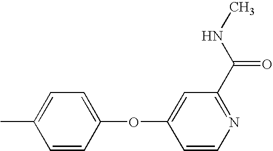 |
| (209) | 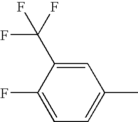 | 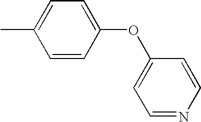 |
| (210) | 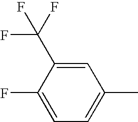 | 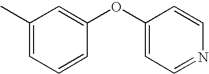 |
| (211) | 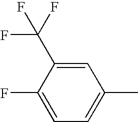 | 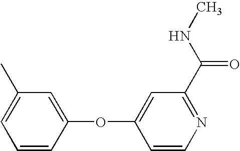 |
| (212) | 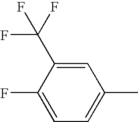 | 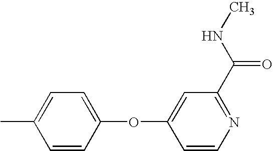 |
| (213) | 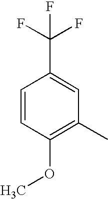 | 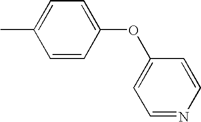 |
| (214) | 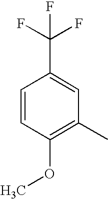 | 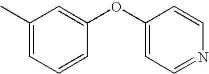 |
| (215) | 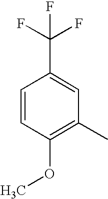 | 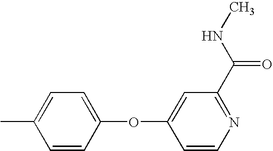 |
| (216) | 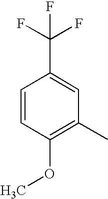 | 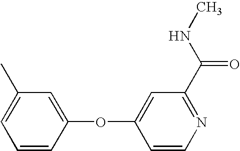 |
| (217) | 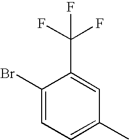 | 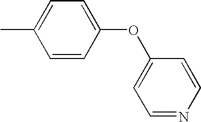 |
| (218) | 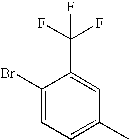 | 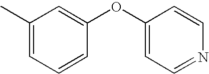 |
| (219) | 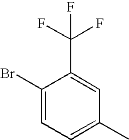 | 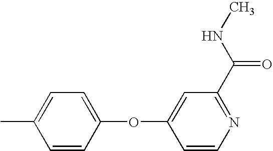 |
| (220) | 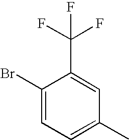 | 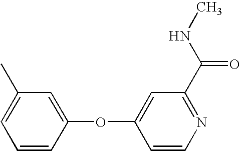 |
| (221) | 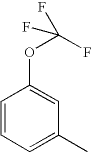 | 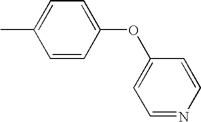 |
| (222) | 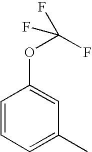 | 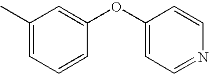 |
| (223) | 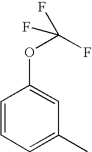 | 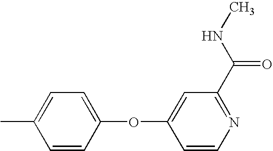 |
| (224) | 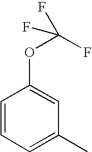 | 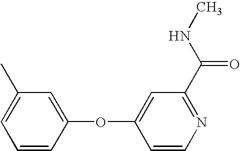 |

The present invention further relates to compounds (225) to (384) as given in the table below:

| | | Mw | tr [min] |
|---|---|---|---|
| 1 | 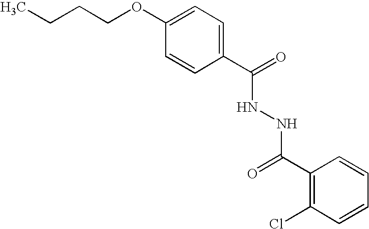 | 346.82 | |
| 2 | 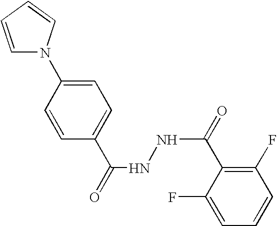 | 341.32 | |
| 3 | 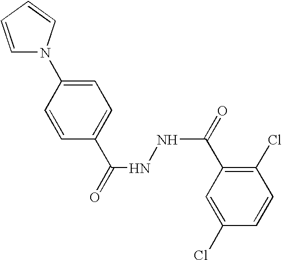 | 374.23 | |
| 4 | 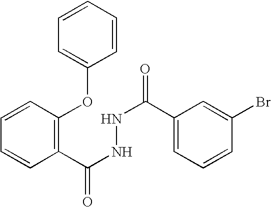 | 411.25 | |
| 5 | 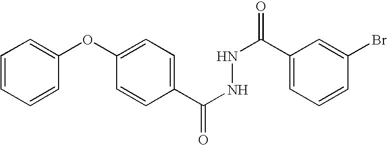 | 411.25 | |
| 6 | 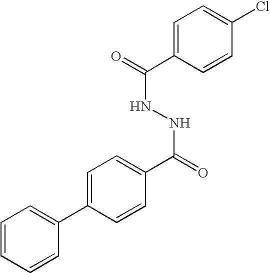 | 350.80 | |
| 7 | 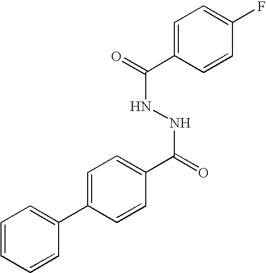 | 334.35 | |
| 8 | 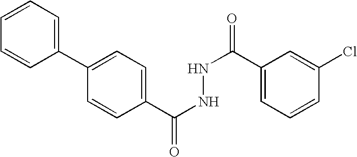 | 350.80 | |
| 9 | 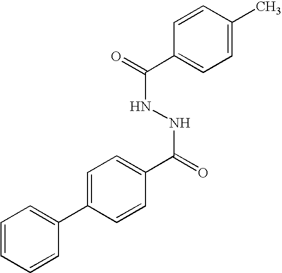 | 330.39 | |
| 10 | 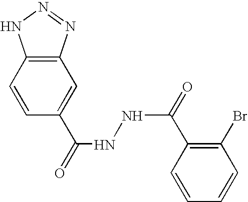 | 360.17 | |
| 11 | 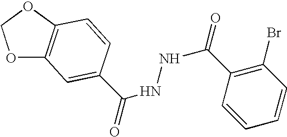 | 363.17 | |
| 12 | 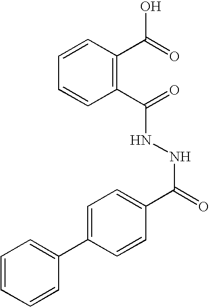 | 360.37 | |
| 13 | 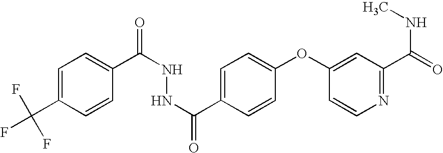 | 458.39 | 2.64^{a)} |
| 14 | 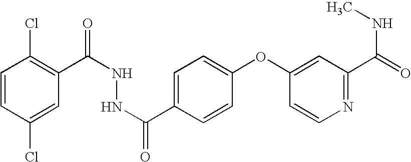 | 459.29 | 2.48^{a)} |
| 15 | 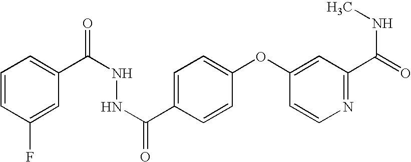 | 408.39 | 1.66^{a)} |
| 16 | 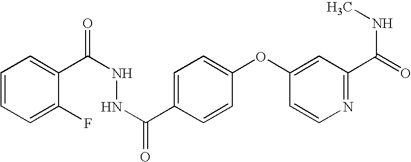 | 408.39 | 1.99^{a)} |
| 17 | 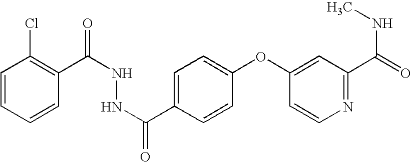 | 424.84 | 1.79^{a)} |
| 18 | 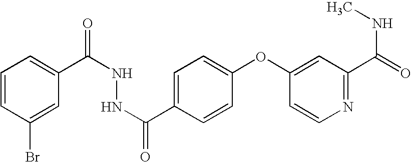 | 469.29 | 2.61^{a)} |
| 19 | 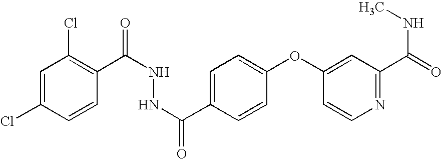 | 459.29 | 2.53^{a)} |
| 20 | 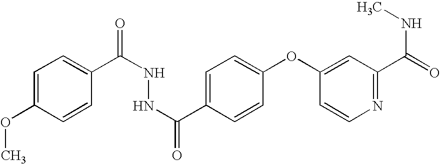 | 420.42 | 1.9^{a)} |
| 21 | 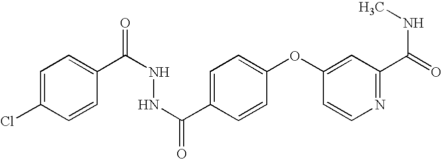 | 424.84 | 2.51^{a)} |
| 22 | 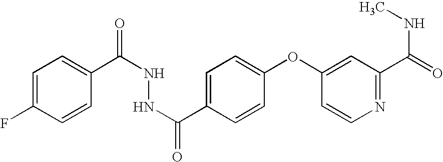 | 408.39 | 1.91^{a)} |
| 23 | 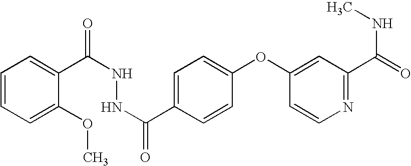 | 420.42 | 2.23^{a)} |
| 24 | 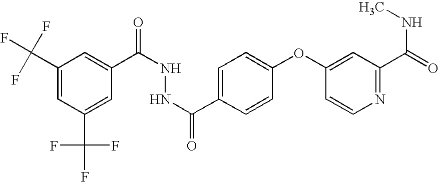 | 526.39 | 3.1^{a)} |
| 25 | 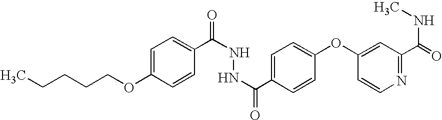 | 476.53 | 3.13^{a)} |
| 26 | 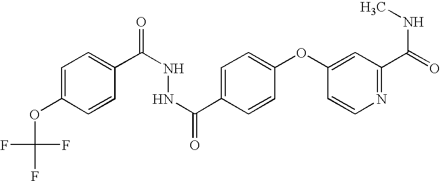 | 474.39 | 2.71^{a)} |
| 27 | 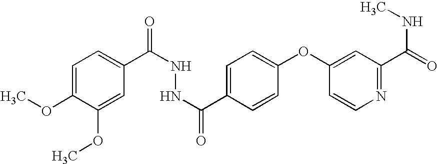 | 450.45 | 1.25^{a)} |
| 28 | 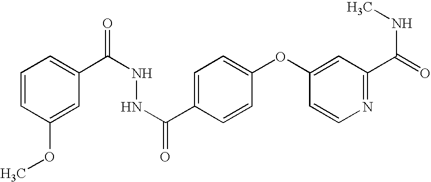 | 420.42 | 2.21^{a)} |
| 29 | 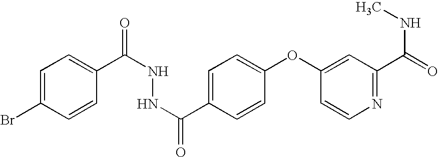 | 469.29 | 2.48^{a)} |
| 30 | 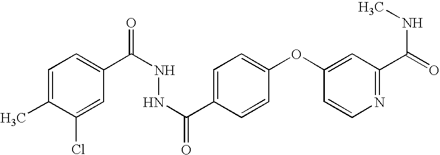 | 438.87 | 2.78^{a)} |
| 31 | 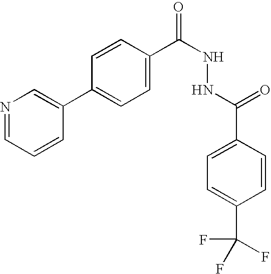 | 385.34 | 3.55^{b)} |
| 32 | 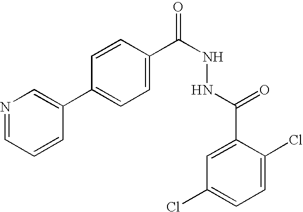 | 386.24 | 3.45^{b)} |
| 33 | 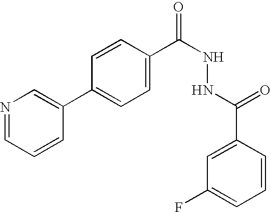 | 335.34 | 3.31^{b)} |
| 34 | 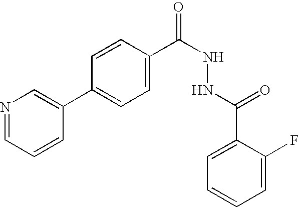 | 335.34 | 3.24^{b)} |
| 35 | 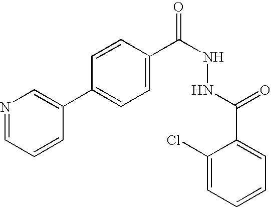 | 351.79 | 3.28^{b)} |
| 36 | 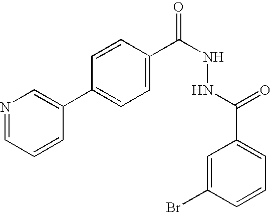 | 396.24 | 3.45^{b)} |
| 37 | 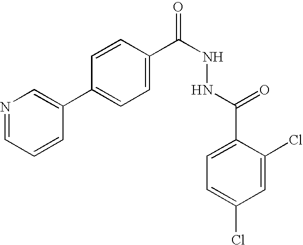 | 386.24 | 3.48^{b)} |
| 38 | 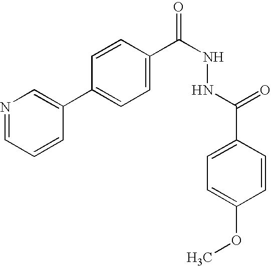 | 347.37 | 3.27^{b)} |
| 39 | 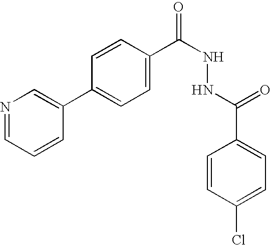 | 351.79 | 3.43^{b)} |
| 40 | 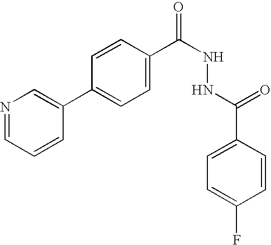 | 335.34 | 3.27^{b)} |
| 41 | 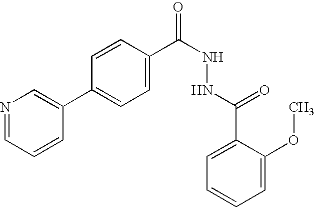 | 347.37 | 3.38^{b)} |
| 42 | 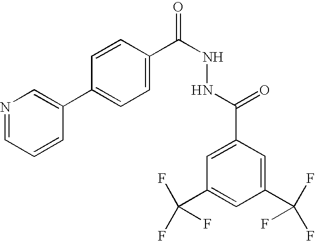 | 453.34 | 3.71^{b)} |
| 43 | 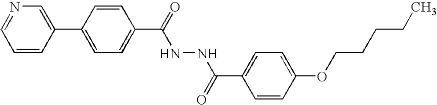 | 403.48 | 3.77^{b)} |
| 44 | 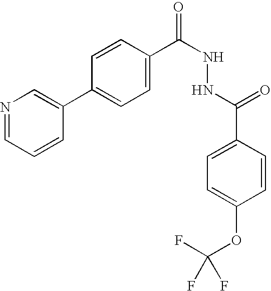 | 401.34 | 3.56^{b)} |
| 45 | 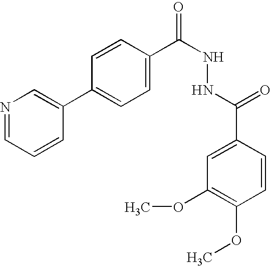 | 377.40 | 3.24^{b)} |
| 46 | 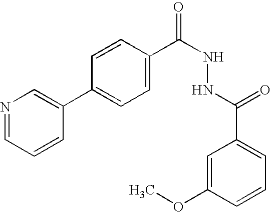 | 347.37 | 3.3^{b)} |
| 47 | 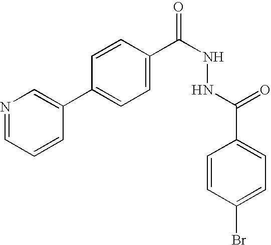 | 396.24 | 3.45^{b)} |
| 48 | 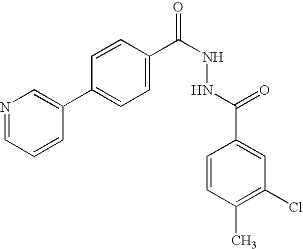 | 365.82 | 3.51^{b)} |
| 49 | 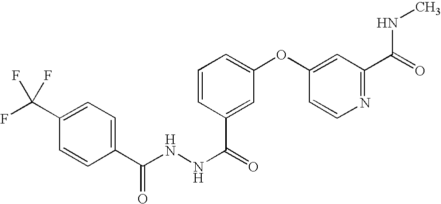 | 458.39 | 2.81^{b)} |
| 50 | 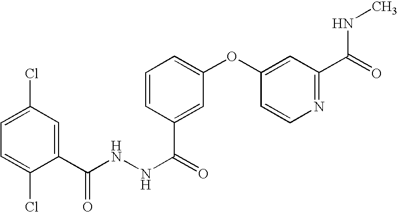 | 459.29 | 2.7^{a)} |
| 51 | 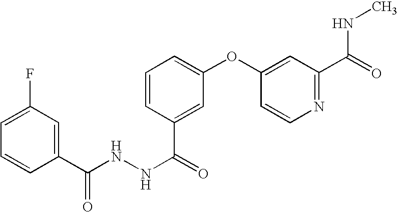 | 408.39 | 2.15^{a)} |
| 52 | 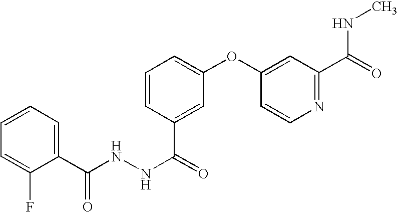 | 408.39 | 1.86^{a)} |
| 53 | 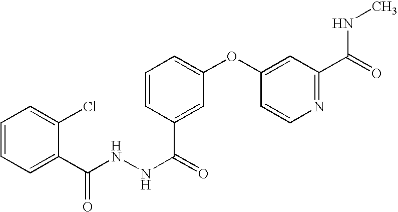 | 424.84 | 2.23^{a)} |
| 54 | 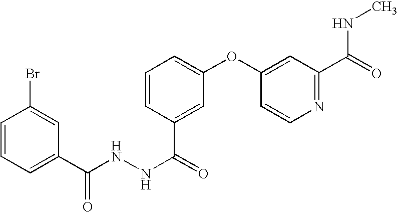 | 469.29 | 2.5^{a)} |
| 55 | 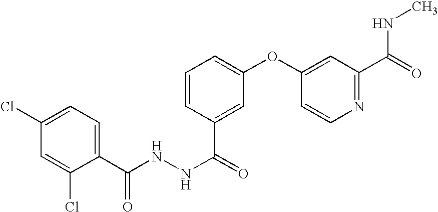 | 459.29 | 2.6^{a)} |
| 56 | 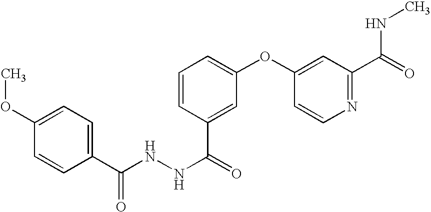 | 420.42 | 1.86^{a)} |
| 57 | 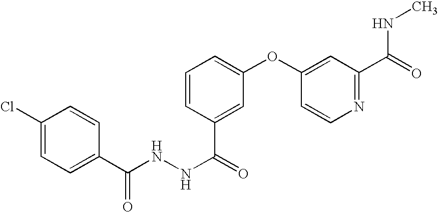 | 424.84 | 2.46^{a)} |
| 58 | 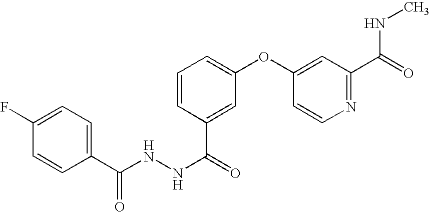 | 408.39 | 2.18^{a)} |
| 59 | 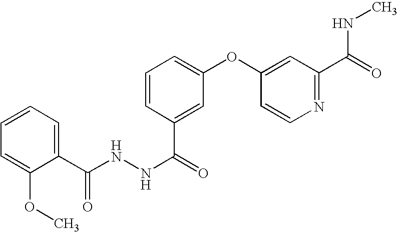 | 420.42 | 2.45^{a)} |
| 60 | 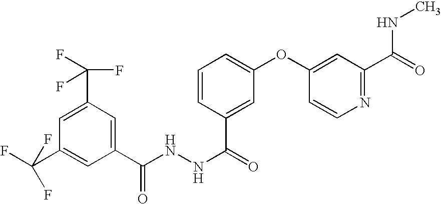 | 526.39 | 3.19^{a)} |
| 61 | 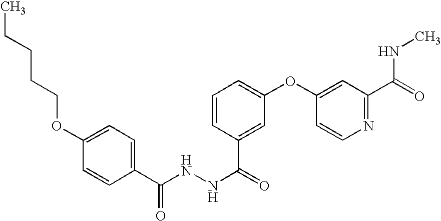 | 476.53 | 3.24^{a)} |
| 62 | 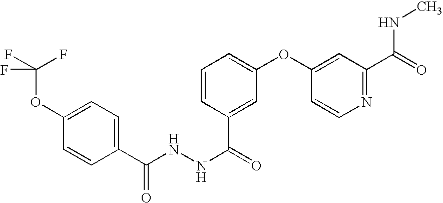 | 474.39 | 2.86^{a)} |
| 63 | 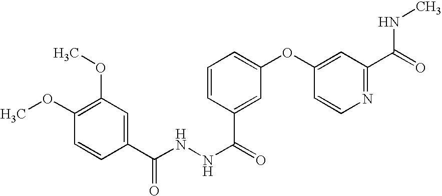 | 450.45 | 1.91^{a)} |
| 64 | 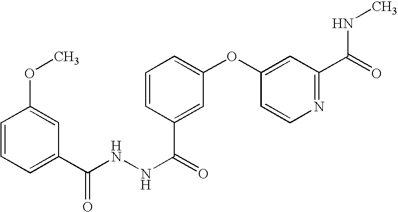 | 420.42 | 2.24^{a)} |
| 65 | 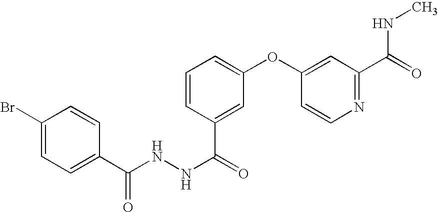 | 469.29 | 2.61^{a)} |
| 66 | 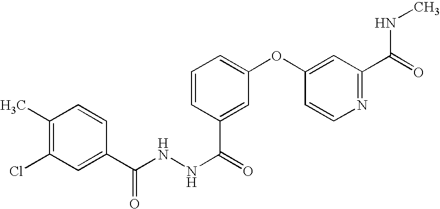 | 438.87 | 2.79^{a)} |
| 67 | 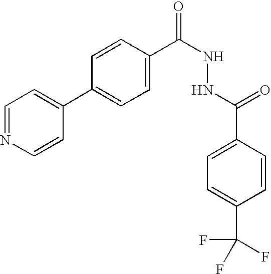 | 385.34 | 3.57^{b)} |
| 68 | 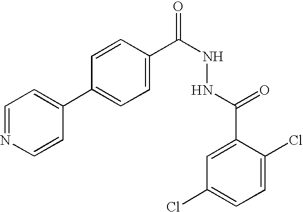 | 386.24 | 3.48^{b)} |
| 69 | 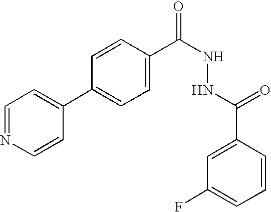 | 335.34 | 3.33^{b)} |
| 70 | 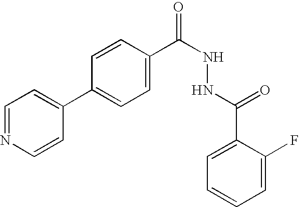 | 335.34 | 3.23^{b)} |
| 71 | 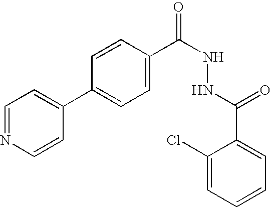 | 351.79 | 3.32^{b)} |
| 72 | 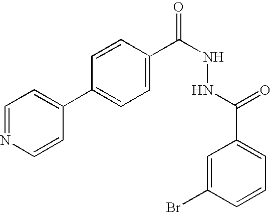 | 396.24 | 3.48^{b)} |
| 73 | 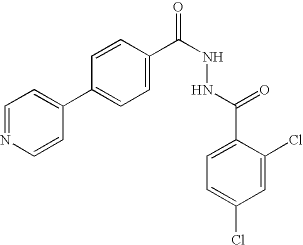 | 386.24 | 3.51^{b)} |
| 74 | 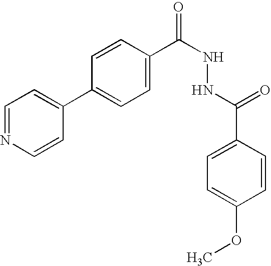 | 347.37 | 3.32^{b)} |
| 75 | 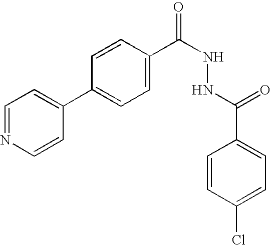 | 351.79 | 3.45^{b)} |
| 76 | 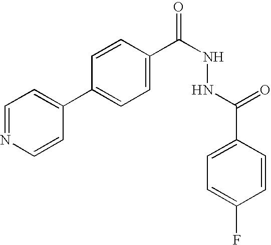 | 335.34 | 3.32^{b)} |
| 77 | 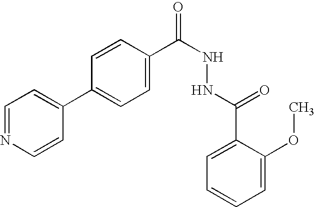 | 347.37 | 3.4^{b)} |
| 78 | 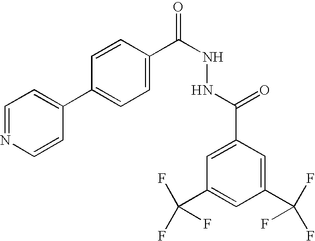 | 453.34 | 3.73^{b)} |
| 79 | 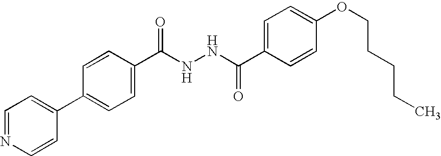 | 403.48 | 3.78^{b)} |
| 80 | 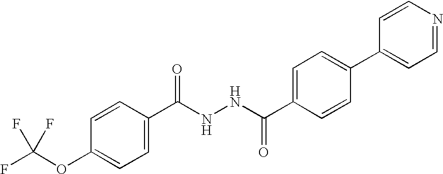 | 401.34 | 3.57^{b)} |
| 81 | 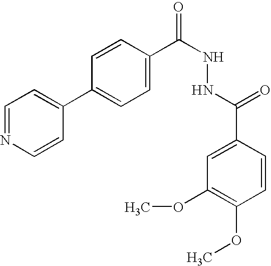 | 377.40 | 3.23^{b)} |
| 82 | 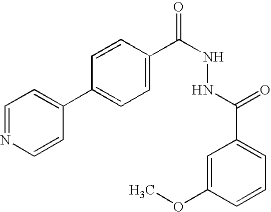 | 347.37 | 3.33^{b)} |
| 83 | 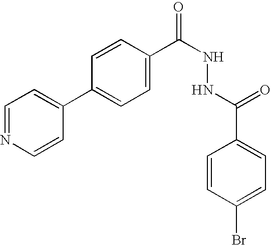 | 396.24 | 3.51^{b)} |
| 84 | 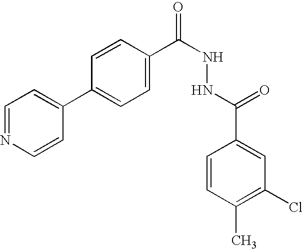 | 365.82 | 3.55^{b)} |
| 85 | 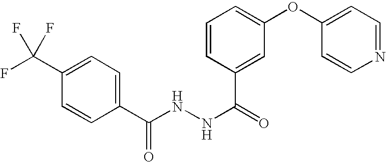 | 401.34 | 3.58^{b)} |
| 86 | 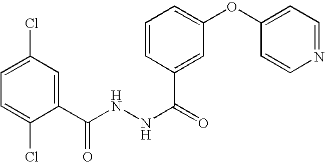 | 402.24 | 3.5^{b)} |
| 87 | 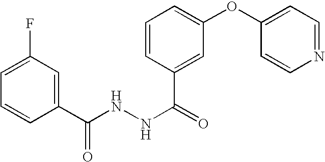 | 351.34 | 3.37^{b)} |
| 88 | 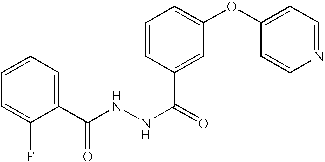 | 351.34 | 3.3^{b)} |
| 89 | 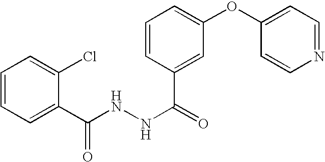 | 367.79 | 3.35^{b)} |
| 90 | 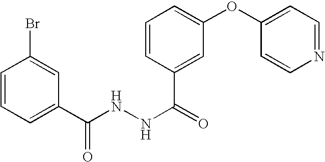 | 412.24 | 3.51^{b)} |
| 91 | 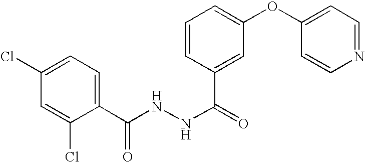 | 402.24 | 3.54^{b)} |
| 92 | 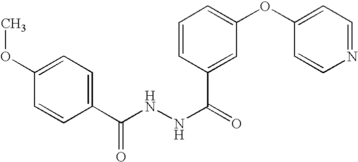 | 363.37 | 3.36^{b)} |
| 93 | 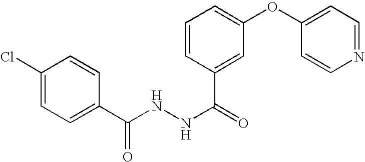 | 367.79 | 3.49^{b)} |
| 94 | 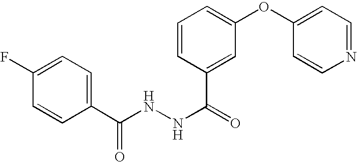 | 351.34 | 3.35^{b)} |
| 95 | 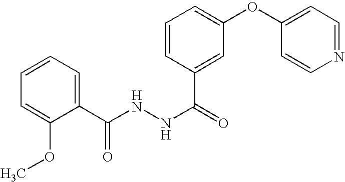 | 363.37 | 3.43^{b)} |
| 96 | 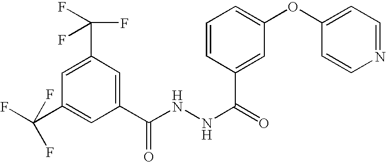 | 469.34 | 3.76^{b)} |
| 97 | 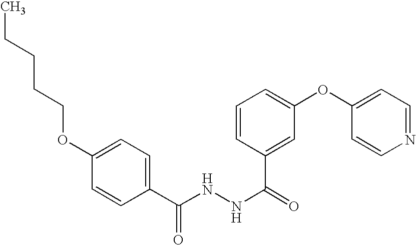 | 419.48 | 3.81^{b)} |
| 98 | 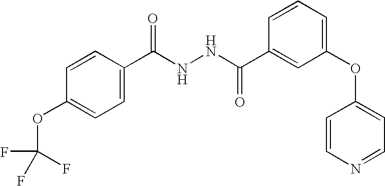 | 417.34 | 3.63^{b)} |
| 99 | 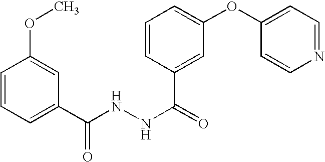 | 363.37 | 3.36^{b)} |
| 100 | 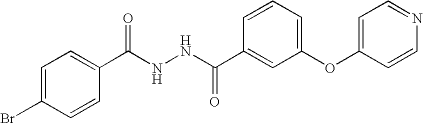 | 412.24 | 3.52^{b)} |
| 101 | 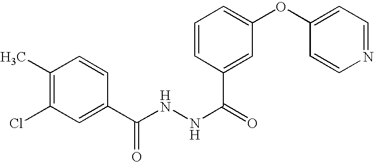 | 381.82 | 3.57^{b)} |
| 102 | 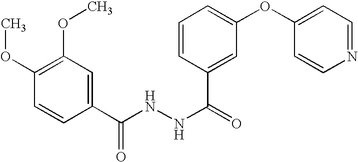 | 393.40 | 3.28^{b)} |
| 103 | 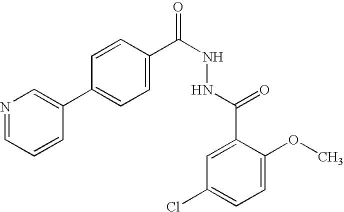 | 381.82 | 1.90^{a)} |
| 104 | 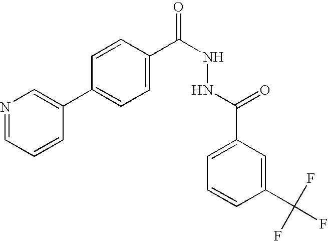 | 385.34 | 1.86^{a)} |
| 105 | 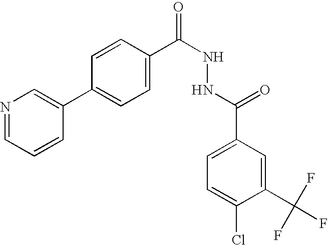 | 419.79 | 2.47^{a)} |
| 106 | 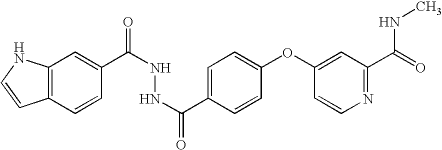 | 429.43 | 2.25^{a)} |
| 107 | 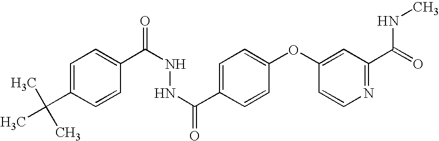 | 446.50 | 3.04^{a)} |
| 108 | 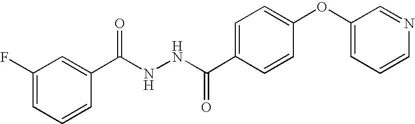 | 351.34 | 3.43^{b)} |
| 109 | 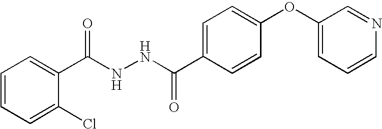 | 367.79 | 0.97^{a)} |
| 110 | 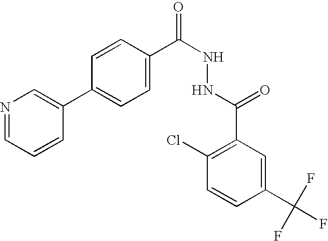 | 419.79 | 3.56^{b)} |
| 111 | 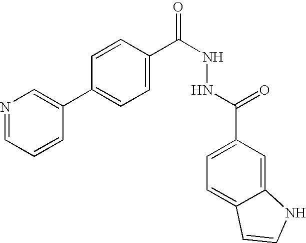 | 356.38 | 3.37^{b)} |
| 112 | 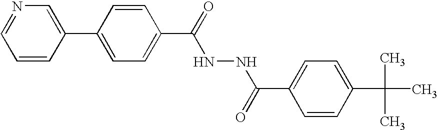 | 373.45 | 3.67^{b)} |
| 113 | 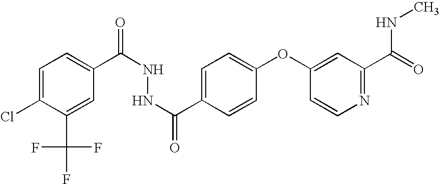 | 492.84 | 3.00^{a)} |
| 114 | 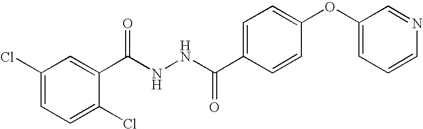 | 402.24 | 3.57^{b)} |
| 115 | 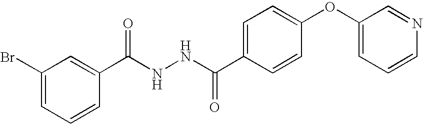 | 412.24 | 1.85^{a)} |
| 116 | 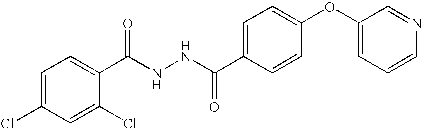 | 402.24 | 3.60^{b)} |
| 117 | 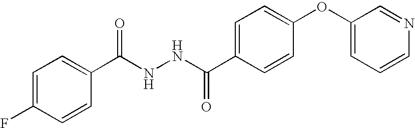 | 351.34 | 3.41^{b)} |
| 118 | 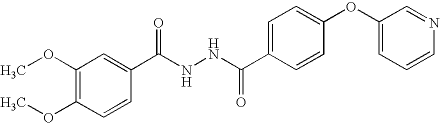 | 393.40 | 3.35^{b)} |
| 119 | 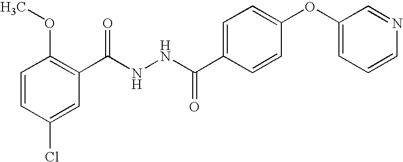 | 397.82 | 3.63^{b)} |
| 120 | 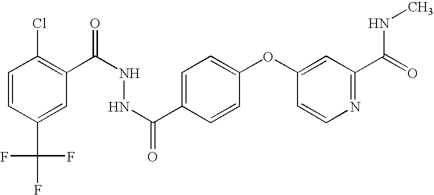 | 492.84 | 2.87^{a)} |
| 121 | 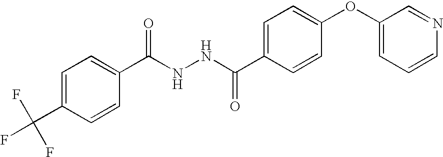 | 401.34 | 2.36^{a)} |
| 122 | 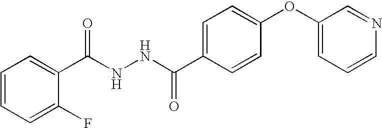 | 351.34 | 3.37^{b)} |
| 123 | 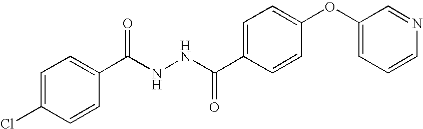 | 367.79 | 3.53^{b)} |
| 124 | 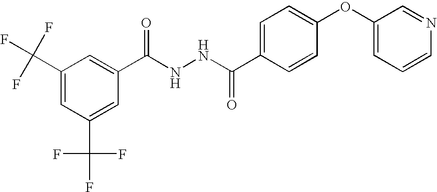 | 469.34 | 3.84^{b)} |
| 125 | 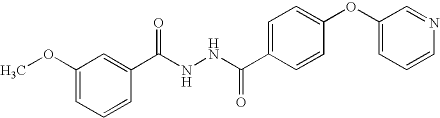 | 363.37 | 3.41^{b)} |
| 126 | 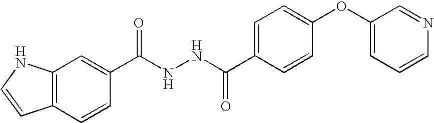 | 372.38 | 3.43^{b)} |
| 127 | 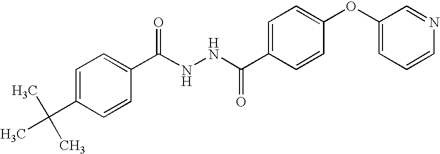 | 389.45 | 3.75^{b)} |
| 128 | 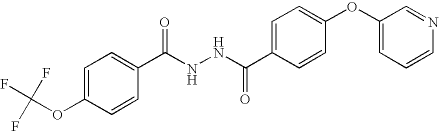 | 417.34 | 3.66^{b)} |
| 129 | 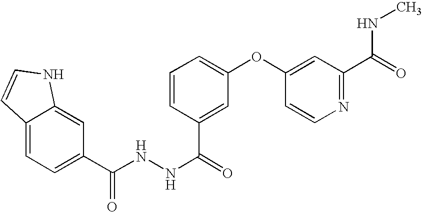 | 429.43 | 3.63^{b)} |
| 130 | 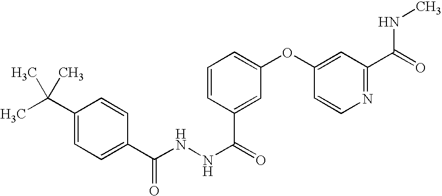 | 446.50 | 3.95^{b)} |
| 131 | 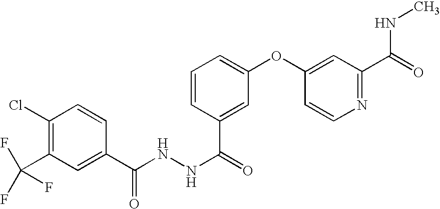 | 492.84 | 3.95^{b)} |
| 132 | 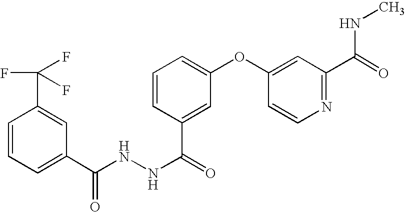 | 458.39 | 3.83^{b)} |
| 133 | 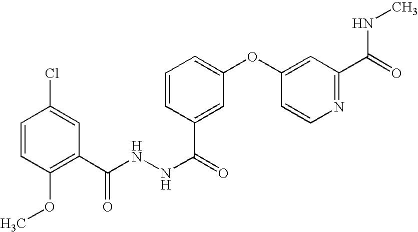 | 454.87 | 3.84^{b)} |
| 134 | 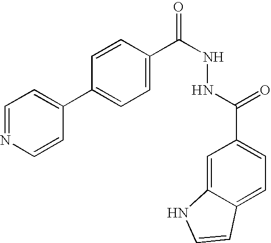 | 356.38 | 3.36^{b)} |
| 135 | 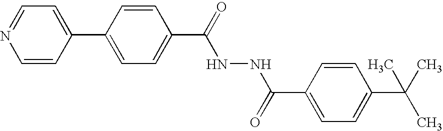 | 373.45 | 3.66^{b)} |
| 136 | 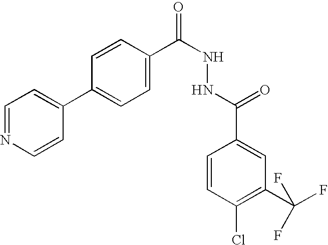 | 419.79 | 3.634⁾ |
| 137 | 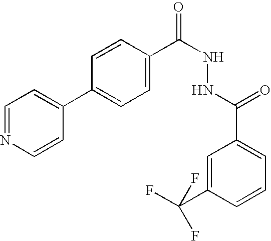 | 385.34 | 3.53^{b)} |
| 138 | 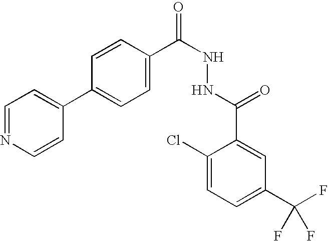 | 419.79 | 3.53^{b)} |
| 139 | 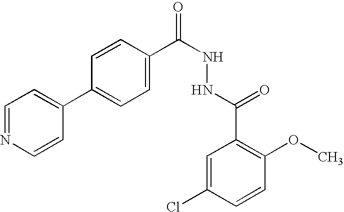 | 381.82 | 3.53^{b)} |
| 140 | 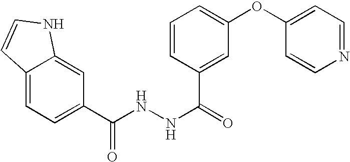 | 372.38 | 3.37^{b)} |
| 141 | 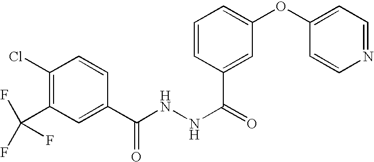 | 435.79 | 3.63^{b)} |
| 142 | 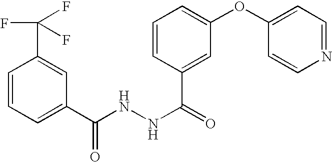 | 401.34 | 3.55^{b)} |
| 143 | 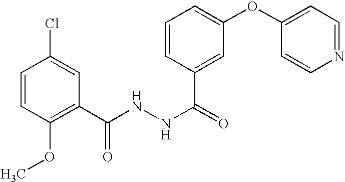 | 397.82 | 3.53^{b)} |
| 144 | 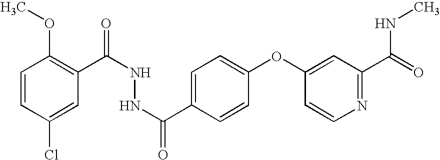 | 454.87 | 2.82^{a)} |
| 145 | 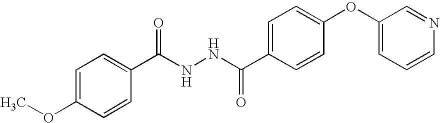 | 363.37 | 3.40^{b)} |
| 146 | 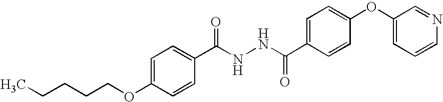 | 419.48 | 3.91^{b)} |
| 147 | 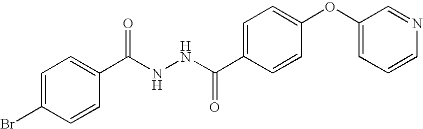 | 412.24 | 3.58^{b)} |
| 148 | 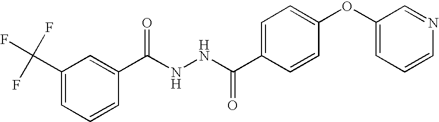 | 401.34 | 3.67^{b)} |
| 149 | 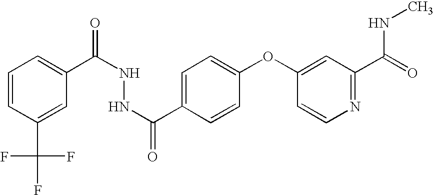 | 458.39 | 2.72^{a)} |
| 150 | 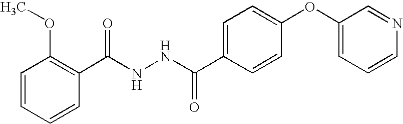 | 363.37 | 3.47^{b)} |
| 151 | 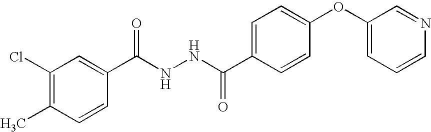 | 381.82 | 3.61^{b)} |
| 152 | 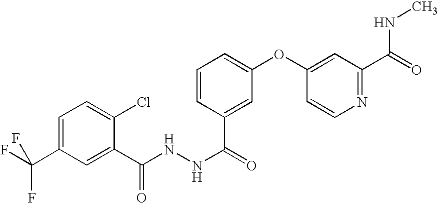 | 492.84 | 3.81^{b)} |
| 153 | 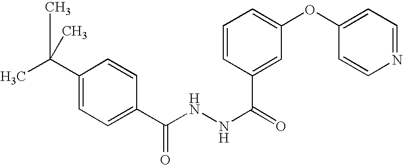 | 389.45 | 3.65^{b)} |
| 154 | 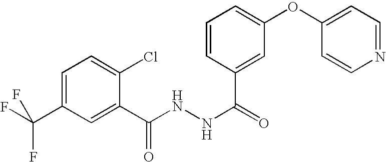 | 435.79 | 3.61^{b)} |
| 155 | 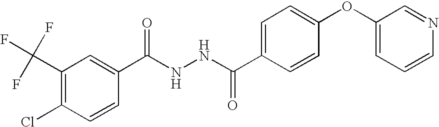 | 435.79 | 3.73^{b)} |
| 156 | 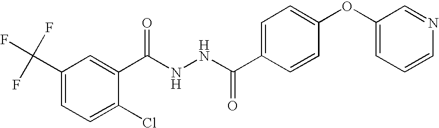 | 435.79 | 3.65^{b)} |
| 157 | 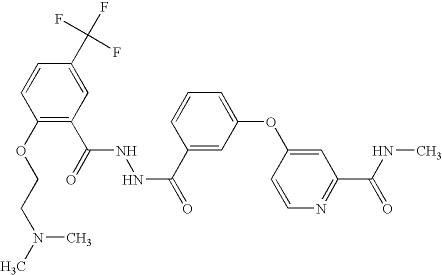 | 545.52 | 2.28^{a)} |
| 158 | 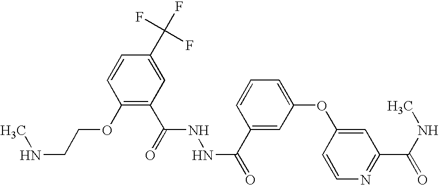 | 531.49 | 3.70^{b)} |
| 159 | 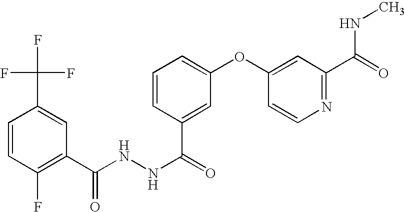 | 476.38 | 2.85^{a)} |

Retention times (tr) were obtained according to the following HPLC metho
Method ^{a)}Gradient: 6 min: flow.: 1.5 ml/min from 80:20 to 0:100-H₂O:ACN water + TFA (0.01% Vol.); acetonitrile + TFA (0.01% Vol.) column: Chromolith SpeedROD RP 18e 50-4.6 wave length: 220 nm
Method ^{b)}Gradient: 6 min: flow: 1.5 ml/min from 100:0 to 0:100-H₂O:ACN water + TFA (0.01% Vol.); acetonitrile + TFA (0.01% Vol.) column: Chromolith SpeedROD RP 18e 50-4.6 wave length: 220 nm

The nomenclature as used herein for defining compounds, especially the compounds according to the invention, is in general based on the rules of the IUPAC-organisation for chemical compounds and especially organic compounds.

In a special embodiment, one or more of the diacylhydrazine derivatives according to sub formulae IIa to IIx and/or compounds (1) to (224), and/or compounds (225) to (384) additionally comprise one or two substituents selected from the group consisting of O(CH₂)ₙNR¹¹R¹², NR¹¹(CH₂)ₙNR¹¹R¹², O(CH₂)ₙOR¹² and NR¹¹(CH₂)ₙOR¹²,

wherein

R¹¹, R¹² are independently selected from a group consisting of H, A, (CH₂)ₘAr³ and (CH₂)ₘHet, or in NR¹¹R¹²,
R¹¹ and R¹² form, together with the N-Atom they are bound to, a 5-, 6- or 7-membered heterocycles which optionally contains 1 or 2 additional hetero atoms, selected from N, O an S, and
n is 1, 2, 3, 4, 5 or 6, preferably 2, 3 or 4.

In this special embodiment, the substituents are preferably selected from the group consisting of HNCH₂CH₂NH₂, OCH₂CH₂NH₂, NHCH₂CH₂OH, OCH₂CH₂NHCH₃, N(CH₃)CH₂CH₂NH₂, HN(CH₃)CH₂CH₂NH, N(CH₃)CH₂CH₂N(CH₃)₂, N(CH₃)CH₂CH₂N(CH₃)₂, N(CH₃)CH₂CH₂OCH₃, OCH₂CH₂N(CH₃)₂, OCH₂CH₂N(CH₂CH₃)₂ and compounds of the formulae 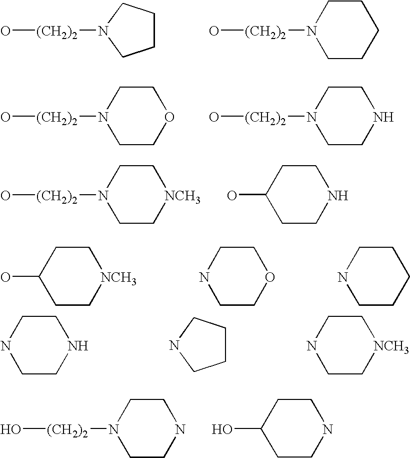
and/or compounds of formulae

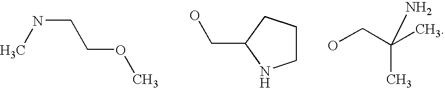

In a further special embodiment, one or more of the diacylhydrazine derivatives according to sub formulae IIa to IIx and/or compounds (1) to (224) and/or compounds (225) to (384) additionally comprise one or two substituents selected from the group consisting of (CH₂)ₙS(O)ᵤNR¹¹R¹² and (CH₂)ₙS(O)ᵤR¹³ wherein R¹¹, R¹² and R¹³ are defined as above and n is as defined above, preferably n is 0, 1 or 2 and especially is 0, and u is preferably 2 or 3. In this embodiment, the residues are preferably selected from SO₂CH₃, SO₂CF₃, OSO₂CH₃, OSO₂CF₃, SO₂NH₂, SO₂NHCH(CH₃)₂, SO₂N(CH₃)₂, SO₂N(CH₂CH₃)₂ and 4-Morpholino-sulfonyl.

In these special embodiments, the additional substituents are preferably bound to one of the aromatic residues directly bound to the diacylhydrazine moiety and/or the pyridinyl residue. More preferably, one or two additional substituents are bound to the residue Ar¹ according to formula II.

Another aspect of the invention relates to a method for producing compounds of formula II, characterised in that
a) A compound of formula III 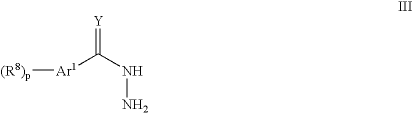
   wherein Y, R⁸, p and Ar¹ are as defined above and below,
   is reacted
b) with a compound of formula IV, 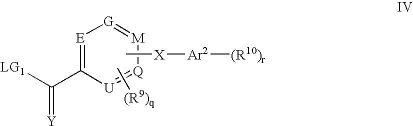
   wherein
   LG₁ is a leaving group, preferably a leaving group selected from OR²⁵, wherein R²⁵ is selected from the group consisting of unsubstituted or substituted aromatic residues, unsubstituted or substituted heteroaromatic residues and (O)₂S—R²⁶ wherein R²⁶ is selected from unsubstituted or substituted aromatic residues and unsubstituted or substituted alkyl residues, and wherein E, G, M, Q, U, R⁹, q, X, Ar², R¹⁰ and r are as defined above and below,
   and optionally
c) isolating and/or treating the compound of formula II obtained by said reaction with an acid, to obtain the salt thereof.

The compounds of the formula I and preferably the compounds of the formula II and also the starting materials for their preparation are prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

If desired, the starting materials can also be formed in situ by not isolating them from the reaction mixture, but instead immediately converting them further into the compounds of the formula I or II, respectively. As an alternative, it is possible to carry out the reaction stepwise.

The compounds of the formula I and especially the compounds of formula II can preferably be obtained by reacting compounds of the formula III with compounds of the formula IV.

In the compounds of formula IV, LG₁ is a suitable leaving group. Suitable leaving groups are known in the art, for example from Houben-Weyl, Methods of Organic chemistry. Preferably, the leaving group is selected from OR²⁵, wherein R²⁵ is selected from the group consisting of unsubstituted or substituted aromatic residues, unsubstituted or substituted heteroaromatic residues and (O)₂S—R²⁶, wherein R²⁶ is selected from unsubstituted or substituted aromatic residues and unsubstituted or substituted alkyl residues.

In compounds of formula IV in which LG₁ is OR²⁵, which option is preferred, R²⁵ is preferably selected from unsubstituted or substituted phenyl moieties, preferably substituted phenyl moieties which comprises one or more nitro groups (—NO₂) and/or one or more sulfonic acid groups (—SO₃H) and/or one or more fluoro groups (F) or salts thereof as substituents. R²⁵ is preferably selected from Ph(Hal)ₓ, wherein x is 1-5 and Hal is selected independently from one another from the group consisting of fluorine, chlorine, bromine and iodine, even more preferably x is 3, 4 or 5 and Hal is fluorine and chlorine and especially preferred Hal is fluorine. Hence, preferably, Ph(Hal)ₓ is selected from Ph(F)ₓ and, especially preferred, Ph(Hal)ₓ is Ph(F)₅.

In compounds of formula IV in which LG₁ is (O)₂S—R²⁶, R²⁶ is selected from unsubstituted or substituted phenyl moieties, preferably alkyl substituted phenyl moieties, and unsubstituted or substituted alkyl residues, preferably unsubstituted or substituted C₁-C₄-alkyl moieties and especially unsubstituted or substituted methyl moieties. Substituted alkyl moieties preferably comprise one or more halogen substituents up to perhalo.

If compounds of formula II are desired wherein Y is different from 0, it can be advantageous however to carry out the reaction of a compound of formula III, wherein Y is O, and a compound of formula IV according to the invention to obtain a compound of formula II, wherein Y is O, and to modify or convert the corresponding C═O group (i.e. the C═Y group, wherein Y is O) in the compound of formula II into a C═NR², C═C(R²²)—NO₂, C═C(R²)—CN or C═C(CN)₂ group according to methods known in the art, for example from Houben-Weyl, Methods of Organic Chemistry.

In detail, the reaction of the compounds of the formula III with the compounds of the formula IV is carried out in the presence or absence of a preferably inert solvent at temperatures between about −20° C. and about 200° C., preferably between 0° C. and 150° C. and especially between room temperature (25°) and 1200. In many cases, it is advantageous to combine one compound of formula III with one compound of formula IV at the lower end of the given temperature range, preferably between −20° C. and 75° C., more preferred between 0° C. and 60° C. and especially between 10° C. and 30° C., for example at about room temperature, and heat the mixture up to a temperature at the upper end of the given temperature range, preferably between 20° C. and 120° C., more preferred between 30° C. and 90° C. and especially between 40° C. and 70° C., for example at about 40° C., at about 50° C. or at about 60° C.

The reaction between the compounds of formula III and compounds of formula IV, wherein LG₁ is OPh(Hal)ₓ, may be carried out in the presence of an acid binding means, for example one or more bases. Suitable acid binding means are known in the art. Preferred as acid binding means are inorganic bases and especially organic bases. Examples for inorganic bases are alkaline or alkaline-earth hydroxides, alkaline or alkaline-earth carbonates and alkaline or alkaline-earth bicarbonates or other salts of a weak acid and alkaline or alkaline-earth metals, preferably of potassium, sodium, calcium or cesium. Examples for organic bases are triethyl amine, diisopropyl ethyl amine (DIPEA), diaza bicyclo undecen (DBU), dimethyl aniline, pyridine or chinoline. If an organic base is used, it is advantageous in general to use a base with a boiling point that is higher than the highest reaction temperature employed during the reaction. Especially preferred as organic bases are DBU and DIPEA. DIPEA is especially preferred in the case that LG is OR²⁵.

Reaction times are generally in the range between some minutes and several days, depending on the reactivity of the respective compounds and the respective reaction conditions. Suitable reaction times can be readily determined by methods known in the art, for example reaction monitoring. Based on the reaction temperatures given above, suitable reaction times generally lie in the range 1 hrs and 120 hrs, preferably 10 h and 100 hrs and especially between 30 hrs and 90 hrs, for example about 48 h, about 50 hrs, about 72 hrs or about 84 hrs.

Preferably, the reaction of the compounds of the formula III with the compounds of the formula IV is carried out in the presence of a suitable solvent, that is preferably inert under the respective reaction conditions. Examples of suitable solvents are hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, dimethylformamide (DMF) or N-methyl pyrrolidinone (NMP); nitriles, such as acetonitrile; sulfoxides, such as dimethyl sulfoxide (DMSO); nitro compounds, such as nitromethane or nitrobenzene; esters, such as ethyl acetate, or mixtures of the said solvents. Polar solvents are in general preferred. Examples for suitable polar solvents are chlorinated hydrocarbons, alcohols, glycol ethers, nitriles, amides and sulfoxides or mixtures thereof. More preferred are chlorinated hydrocarbons, especially dichloromethane, sulfoxides, especially DMSO, and ketones, especially DMF.

Preferably, the reaction between a compound of formula III and a compound of formula IV is carried out in an inert solvent, preferably a solvent boiling at higher temperatures, at the lower end of the given temperature range, for example in a ketone, for example DMF, in a temperature range between 40° C. and 70° C., preferably at about 55° C. Reaction times generally lie in the range of 30 hours to 90 hrs, for example at about 72 hrs. Preferably, no acid binding means is present.

In general, the compounds of formula III and/or formula IV are new, but can be prepared according to methods known in the art.

Therefore, a further object of the instant invention are the compounds of the formula III and IV.

The compounds of formula III can be obtained according to methods known in the art. In an advantageous manner, they can be readily obtained by one or more of the reaction routes given below:

Compounds of formula III can be readily obtained from suitable educts according to known procedures for producing aryl hydrazides. For example, the corresponding lower alkyl esters, preferably methyl esters, can be reacted with hydrazine monohydrate. If desired, compounds of formula III, wherein Y is O can be readily derivatized to compounds of formula III, wherein Y is S, according to procedures known in the art.

The compounds of formula IV can be readily obtained in an advantageous manner by reacting a compound of formula V, 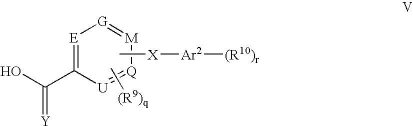
wherein Y, E, G, M, Q, U, R⁹ and q are as defined above/below,
with a compound of formula VI,
LG₁-H VI
wherein LG₁ is a leaving group as defined above, in a condensation reaction and
optionally isolating the reaction product.

Methods and reaction conditions for condensation reactions are known in the art. In general, it is advantageous to carry out the reaction in the presence of a water binding agent, for example dicyclohexyl carbodiimide. In general, such condensation reactions are carried out in a suitable solvent. Suitable solvents for condensation reactions are known in the art. Suitable solvents, for example, are inert solvents, preferably ethers, especially dioxane. Preferably, the reaction is carried out in a inert gas atmosphere. In general, the condensation reactions are carried out at about normal pressure or elevated pressure, for example between normal pressure and 10 bar pressure, preferably at normal pressure. The reaction is usually carried out in the temperature range between −20° C. and 120° C., preferably +0° C. and 50° C., for example at room temperature. Generally, suitable reaction times for the condensation reaction range between 1 hrs and 100 hrs, preferably 5 h and 50 hrs and especially between 10 hrs and 20 hrs, for example about 15 hrs.

Where the reactants are readily oxidized it may advisable to carry out the reaction in an inert gas atmosphere.

Where X is O, the compounds of formula V can be readily obtained in an advantageous manner by reacting a compound of formula VIIa, 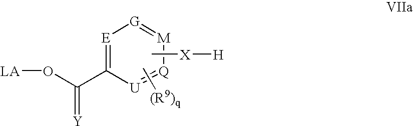
wherein Y, E, G, M, Q, U, R⁹ and q are as defined above/below,
LA is H or a lower alkyl radical, for example methyl, ethyl, n-propyl, iso-propyl, n-butyl, 2-butyl or tert-butyl, preferably ethyl, and
X is O,
   with a compound of formula VIII,
   L¹⁰-Ar² [symbol] R¹⁰)ᵣ VII
   wherein L¹⁰ is preferably Cl, Br, I or diazonium moiety, more preferred Cl, Br or I and even more preferred Br and Cl, in a condensation reaction and
   optionally isolating the reaction product.

The reaction between the compound of formula VIIa and VIII is preferably carried out in the temperature range between 0° C. and 250° C., more preferred 50° C. and 220° C., for example at about 90° C., at about 120° C., at about 160° C., at about 180° C. or at about 200°. Reaction times depend on the respective reactants and the respective reaction temperature, but generally lie in the range between 10 min and 36 hrs, preferably between 60 min and 24 hrs, more preferably 3 h and 20 hrs for example about 6 hrs, about 12 hrs, about 15 hrs or about 18 hrs.

The reaction can be carried out in the absence or the presence of a solvent, preferable a solvent that is inert under the respective reaction conditions. Suitable inert solvents for carrying out the reaction are known in the art. Examples for suitable solvents are high aliphatic hydrocarbons, aromatic carbons, for example toluene and xylenes, high boiling chlorinated hydrocarbons, such as dichlormethane, trichloromethane trichloroethylene, tetrachloroethanes, pentachloroethanes and hexachloroethanes; ethers, such as diethylether, tert.-butyl methyl ether, ethylene glycol and propylene glycols; glycol ethers, such as ethylene glycol monomethyl or monoethyl ether or ethylene glycol dimethyl ether (diglyme); nitriles, such as acetonitrile, amides such as acetamide, dimethyacetamide, dimethylformamide (DMF) or N-methylpyrrolidinone (NMP); sulfoxides, such as dimethyl sulfoxide (DMSO); or mixtures of the said solvents.

In many cases, it is advantageous to carry out the reaction in the presence of a catalyst. Suitable catalysts are known in the art.

Often, it is advantageous to carry out the reaction in the presence of an acid binding means, preferably an organic base as described above and more preferred an inorganic base. Preferred inorganic bases are K₂CO₃, Na₂CO₃, MgCO₃, CaCO₃, NaOH and KOH, especially preferred is K₂CO₃.

Where X is a bond, the compounds of formula V can be readily obtained in an advantageous manner by reacting a compound of formula VIIb, 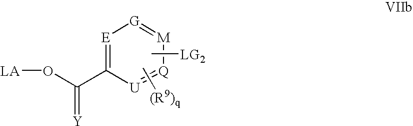
wherein Y, E, G, M, Q, U, R⁹, LA and q are as defined above/below,
X is O and
LG₂ is a suitable leaving group known in the art, preferably LG₂ is a boronic acid residue B(OH)₂.
   with a compound of formula VIII,
   L¹⁰-Ar² [symbol] R¹⁰)ᵣ VIII
   wherein L¹⁰ is preferably Cl, Br, I or diazonium moiety, more preferred Cl, Br or I and even more preferred Br and Cl, in a condensation reaction and
   optionally isolating the reaction product.

The reaction between the compound of formula VIIb and VIII is preferably carried out in the temperature range between 0° C. and 160° C., more preferred 40° C. and 120° C., for example at about 60° C., at about 80° C., or at about 100° C. Reaction times depend on the respective reactants and the respective reaction temperature, but generally lie in the range between 10 min and 36 hrs, preferably between 60 min and 24 hrs, more preferably 3 h and 20 hrs for example about 6 hrs, about 12 hrs, about 15 hrs or about 18 hrs.

In many cases, it is advantageous to carry out the reaction in the presence of a catalyst. Suitable catalysts are known in the art.

Where the reactants are readily oxidized it may advisable to carry out the reaction in an inert gas atmosphere.

The reaction can be carried out in the absence or the presence of a solvent, preferable a solvent that is inert under the respective reaction conditions. Suitable inert solvents for carrying out the reaction are known in the art. Examples for suitable solvents are high aliphatic hydrocarbons, aromatic carbons, for example toluene and xylenes, high boiling chlorinated hydrocarbons, such as dichlormethane, trichloromethane trichloroethylene, tetrachloroethanes, pentachloroethanes and hexachloroethanes; ethers, such as diethylether, tert.-butyl methyl ether, ethylene glycol and propylene glycols; glycol ethers, such as ethylene glycol monomethyl or monoethyl ether or ethylene glycol dimethyl ether (diglyme); nitriles, such as acetonitrile, amides such as acetamide, dimethyacetamide, dimethylformamide (DMF) or N-methylpyrrolidinone (NMP); sulfoxides, such as dimethyl sulfoxide (DMSO); or mixtures of the said solvents.

Often, it is advantageous to carry out the reaction in the presence of an acid binding means, preferably an organic base as described above and more preferred an inorganic base. Preferred inorganic bases are K₂CO₃, Na₂CO₃, MgCO₃, CaCO₃, NaOH and KOH, especially preferred is K₂CO₃.

Independently of the chosen reaction route, it is in many cases possible or even feasible to introduce residues R⁸, R⁹ and/or R¹⁰ into one or more of the compounds described above, or, if the compound already comprises one or more residues R⁸, R⁹ and/or R¹⁰, to introduce additional residues R⁸, R⁹ and/or R¹⁰ into said compound. The introduction of additional residues can be readily performed by methods known in the art and especially by aromatic substitution, for example nucleophilic aromatic substitution or electrophilic aromatic substitution. For example, in compounds comprising Ar¹, wherein Ar¹ comprises one or more halogen and preferably fluorine substituents, one or more of the halogen/fluorine substituents can be easily substituted by hydroxy, thio and/or amino substituted hydrocarbons, preferably selected from the group consisting of HO(CH₂)ₙNR¹¹R¹², HO(CH₂)ₙO(CH₂)ₖNR¹¹R¹², HO(CH₂)ₙNR¹¹(CH₂)ₖOR¹², HO(CH₂)ₙNR¹¹(CH₂)ₖNR¹¹R¹², HO(CH₂)ₙCOOR³, HO(CH₂)ₙS(O)ᵤR¹³HNR¹¹(CH₂)ₙNR¹¹R¹², HNR¹¹(CH₂)ₙO(CH₂)ₖNR¹¹R¹², HNR¹¹(CH₂)ₙNR¹¹(CH₂)ₖOR², HNR¹¹(CH₂)ₙNR¹¹(CH₂)ₖNR¹¹R¹², HNR¹¹(CH₂)ₙCOOR¹² and HNR¹¹(CH₂)ₙS(O)ᵤR¹³ wherein R¹¹, R¹² and R¹³ are defined as above and n is as defined above, preferably n is 0, 1 or 2 and especially is 0, k is 1 to 4 and preferably 1 or 2, and u is preferably 2. In this embodiment R¹¹, R¹² and R¹³ are more preferably selected independently from each other from the group consisting of H, methyl and ethyl. Even more preferred, the hydroxy, thio and/or amino substituted hydrocarbons are selected from the group consisting of NH₃, HN(CH₃)₂, NH₂CH₃, HN(C₂H₅)₂, H₂NCH₂CH₂NH₂, HOCH₂CH₂NH₂, HOCH₂CH₂NHCH₃, HN(CH₃)CH₂CH₂NH₂, HN(CH₃)CH₂CH₂N(CH₃)₂, HN(CH₃)CH₂CH₂N(CH₃)₂, HN(CH₃)CH₂CH₂OCH₃, HOCH₂CH₂N(CH₃)₂, HOCH₂CH₂N(CH₂CH₃)₂, HSCH₃, HSC₂H₅, and compounds of the formulae 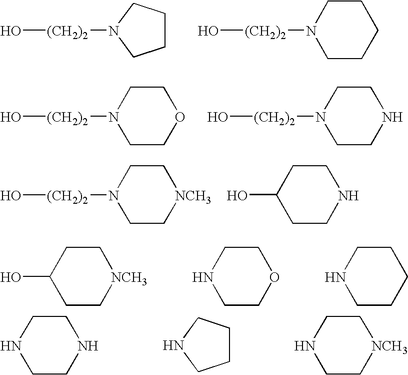
or salts and especially metal salts thereof.

On the other hand, it is in many cases possible or even feasible to modify or derivatize one or more of the residue is R⁸, R⁹ and R¹⁰ into residues R⁸, R⁹ and/or R¹⁰ other than the ones originally present. For example, CH₃— groups can be oxidised into aldehyde groups or carbonic acid groups, thio atom containing groups, for example S-alkyl or S-aryl groups, can be oxidised into SO₂-alkyl or SO₂-aryl groups, respectively, carbonic acid groups can be derivatized to carbonic acid ester groups or carbon amide groups and carbonic acid ester groups or carbon amide groups can be hydrolysed into the corresponding carbonic acid groups. Methods for performing such modifications or derivatizations are known in the art, for example from Houben-Weyl, Methods of Organic Chemistry.

Every reaction step described herein can optionally be followed by one or more working up procedures and/or isolating procedures. Suitable such procedures are known in the art, for example from standard works, such as Houben-Weyl, Methoden der organischen Chemie [Methods of Organic Chemistry], Georg-Thieme-Verlag, Stuttgart). Examples for such procedures include, but are not limited to evaporating a solvent, distilling, crystallization, fractionised crystallization, extraction procedures, washing procedures, digesting procedures, filtration procedures, chromatography, chromatography by HPLC and drying procedures, especially drying procedures in vacuo and/or elevated temperature.

A base of the formula I or the formula II can be converted into the associated acid-addition salt using an acid, for example by reaction of equivalent amounts of the base and the acid in a preferably inert solvent, such as ethanol, followed by evaporation. Suitable acids for this reaction are, in particular, those which give physiologically acceptable salts. Thus, it is possible to use inorganic acids, for example sulfuric acid, sulfurous acid, dithionic acid, nitric acid, hydrohalic acids, such as hydrochloric acid or hydrobromic acid, phosphoric acids, such as, for example, orthophosphoric acid, sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, hexanoic acid, octanoic acid, decanoic acid, hexadecanoic acid, octadecanoic acid, pivalic acid, diethylacetic acid, malonic acid, succinic acid pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, trimethoxybenzoic acid, adamantanecarboxylic acid, p-toluenesulfonic acid, glycolic acid, embonic acid, chlorophenoxyacetic acid, aspartic acid, glutamic acid, proline, glyoxylic acid, palmitic acid, parachlorophenoxyisobutyric acid, cyclohexanecarboxylic acid, glucose 1-phosphate, naphthalenemono- and -disulfonic acids or laurylsulfuric acid. Salts with physiologically unacceptable acids, for example picrates, can be used to isolate and/or purify the compounds of the formula I. On the other hand, compounds of the formula I can be converted into the corresponding metal salts, in particular alkali metal salts or alkaline earth metal salts, or into the corresponding ammonium salts, using bases (for example sodium hydroxide, potassium hydroxide, sodium carbonate or potassium carbonate). Suitable salts are furthermore substituted ammonium salts, for example the dimethyl-, diethyl- and diisopropylammonium salts, monoethanol-, diethanol- and diisopropanolammonium salts, cyclohexyl- and dicyclohexylammonium salts, dibenzylethylenediammonium salts, furthermore, for example, salts with arginine or lysine.

On the other hand, if desired, the free bases of the formula I or the formula II can be liberated from their salts using bases (for example sodium hydroxide, potassium hydroxide, sodium carbonate or potassium carbonate).

The invention relates to compounds of the formula I and of the formula II and physiologically acceptable salts and solvates thereof as medicaments.

The invention also relates to the compounds for the formula I and of the formula II and physiologically acceptable salts and solvates thereof as kinase inhibitors.

The invention furthermore relates to the use of the compounds of the formula I and/or physiologically acceptable salts and/or solvates thereof for the preparation of pharmaceutical compositions and/or pharmaceutical preparations, in particular by non-chemical methods. The invention furthermore relates to the use of the compounds of the formula II and/or physiologically acceptable salts and/or solvates thereof for the preparation of pharmaceutical compositions and/or pharmaceutical preparations, in particular by non-chemical methods. In this cases, one or more compounds according to the invention can be converted into a suitable dosage form together with at least one solid, liquid and/or semi-liquid excipient or adjuvant and, if desired, in combination with one or more further active ingredients.

The invention further relates to the use of one or more of the compounds according to the invention, selected from the group consisting of compounds of the formula I as free bases, solvates of compounds of the formula I, salts of compounds of formula I, of compounds of the formula II as free bases, solvates of compounds of the formula II and salts of compounds of formula II, for the production of pharmaceutical compositions and/or pharmaceutical preparations, in particular by a non-chemical route. In general, non-chemical routes for the production of pharmaceutical compositions and/or pharmaceutical preparations comprise processing steps on suitable mechanical means known in the art that transfer one or more compounds according to the invention into a dosage form suitable for administration to a patient in need of such a treatment. Usually, the transfer of one or more compounds according to the invention into such a dosage form comprises the addition of one or more compounds, selected from the group consisting of carriers, excipients, auxiliaries and pharmaceutical active ingredients other than the compounds according to the invention. Suitable processing steps include, but are not limited to combining, milling, mixing, granulating, dissolving, dispersing, homogenizing, casting and/or compressing the respective active and non-active ingredients. In this respect, active ingredients are preferably at least one compound according to this invention and one or more additional compounds other than the compounds according to the invention, which show valuable pharmaceutical properties, preferably those pharmaceutical active agents other than the compounds according to invention which are disclosed herein.

The process for preparing pharmaceutical compositions and/or pharmaceutical preparations preferably comprises one or more processing steps, selected from the group consisting of combining, milling, mixing, granulating, dissolving, dispersing, homogenizing and compressing. The one or more processing steps are preferably performed on one or more of the ingredients which are to form the pharmaceutical composition and/or pharmaceutical preparation preferably according to invention. Even more preferred, said processing steps are performed on two or more of the ingredients which are to form the pharmaceutical composition and/or pharmaceutical preparation, said ingredients comprising one or more compounds according to the invention and, additionally, one or more compounds, preferably selected from the group consisting of active ingredients other than the compounds according to the invention, excipients, auxiliaries, adjuvants and carriers. Mechanical means for performing said processing steps are known in the art, for example from Ullmann's Encyclopedia of Industrial Chemistry, 5th Edition.

Preferably, one or more compounds according to the invention are converted into a suitable dosage form together with at least one compound selected from the group consisting of excipients, auxiliaries, adjuvants and carriers, especially solid, liquid and/or semi-liquid excipients, auxiliaries, adjuvants and carriers, and, if desired, in combination with one or more further active ingredients.

Suitable dosage forms include, but are not limited to tablets, capsules, semi-solids, suppositories, aerosols, which can be produced according to methods known in the art, for example as described below:
tablets mixing of active ingredient/s and auxiliaries, compression of said mixture into tablets (direct compression), optionally granulation of part of mixture before compression
capsules mixing of active ingredient/s and auxiliaries to obtain a flowable powder, optionally granulating powder, filling powders/granulate into opened capsules, capping of capsules
semi-solids
(ointments, gels, creams) dissolving/dispersing active ingredient/s in an aqueous or fatty carrier;
   subsequent mixing of aqueous/fatty phase with complementary fatty resp. aqueous phase, homogenisation (creams only)
suppositories
(rectal and vaginal) dissolving/dispersing active ingredient/s in carrier material liquified by heat (rectal: carrier material normally a wax; vaginal: carrier normally a heated solution of a gelling agent), casting said mixture into suppository forms, annealing and withdrawal suppositories from the forms
aerosols: dispersing/dissolving active agent/s in a propellant, bottling said mixture into an atomizer

The invention thus relates to pharmaceutical compositions and/or pharmaceutical preparations comprising at least one compound of the formula I and/or one of its physiologically acceptable salts and/or solvates and especially to pharmaceutical compositions and/or pharmaceutical preparations comprising at least one compound of the formula II and/or one of its physiologically acceptable salts and/or solvates.

Preferably, the pharmaceutical compositions and/or pharmaceutical preparations according to the invention contain a therapeutic effective amount of one or more compounds according to the invention. Said therapeutic effective amount of one or more of the compounds according to the invention is known to the skilled artisan or can be easily determined by standard methods known in the art. For example, the compounds according to the invention can be administered to a patient in an analogous manner to other compounds that are effective as raf-kinase inhibitors, especially in an analogous manner to the compounds described in WO 00/42012 (Bayer). Usually, suitable doses that are therapeutically effective lie in the range between 0.0005 mg and 1000 mg, preferably between 0.005 mg and 500 mg and especially between 0.5 and 100 mg per dose unit. The daily dose comprises preferably more than 0.001 mg, more preferred more than 0.01 milligram, even more preferred more than 0.1 mg and especially more than 1.0 mg, for example more than 2.0 mg, more than 5 mg, more than 10 mg, more than 20 mg, more than 50 mg or more than 100 mg, and preferably less than 1500 mg, more preferred less than 750 mg, even more preferred less than 500 mg, for example less than 400 mg, less than 250 mg, less than 150 mg, less than 100 mg, less than 50 mg or less than 10 mg.

The specific dose for the individual patient depends, however, on the multitude of factors, for example on the efficacy of the specific compounds employed, on the age, body weight, general state of health, the sex, the kind of diet, on the time and route of administration, on the excretion rate, the kind of administration and the dosage form to be administered, the pharmaceutical combination and severity of the particular disorder to which the therapy relates. The specific therapeutic effective dose for the individual patient can readily be determined by routine experimentation, for example by the doctor or physician which advises or attends the therapeutic treatment.

However, the specific dose for each patient depends on a wide variety of factors, for example on the efficacy of the specific compound employed, on the age, body weight, general state of health, sex, on the diet, on the time and method of administration, on the rate of excretion, medicament combination and severity of the particular illness to which the therapy applies. Parenteral administration is preferred. Oral administration is especially preferred.

These compositions and/or preparations can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatine, carbohydrates, such as lactose or starch, magnesium stearate, talc or vaseline. Examples for suitable dosage forms, which are especially suitable for oral administration are, in particular, tablets, pills, coated tablets, capsulees, powders, granules, syrups, juices or drops. Further examples for suitable dosage forms, which are especially suitable for rectal administration are suppositories, further examples for suitable dosage forms, which are especially suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations. The compositions and/or preparations indicated may be sterilized and/or comprise assistants, such as lubricants, preservatives, stabilizers and/or wetting agents, emulsifiers, salts for modifying the osmotic pressure, buffer substances, dyes and flavors and/or one or more further active ingredients, for example one or more vitamins.

For administration as an inhalation spray, it is possible to use sprays in which the active ingredient is either dissolved or suspended in a propellant gas or propellant gas mixture (for example CO₂ or chlorofluorocarbons). The active ingredient is advantageously used here in micronized form, in which case one or more additional physiologically acceptable solvents may be present, for example ethanol. Inhalation solutions can be administered with the aid of conventional inhalers.

The compounds of the formula I and their physiologically acceptable salts and solvates and especially the compounds of formula II and their physiologically acceptable salts and solvates can be employed for combating one or more diseases, for example allergic diseases, psoriasis and other skin diseases, especially melanoma, autoimmune diseases, such as, for example, rheumatoid arthritis, multiple sclerosis, Crohn's disease, diabetes mellitus or ulcerative colitis.

In general, the substances according to the invention are preferably administered in doses between”1 and 500 mg, in particular between 5 and 100 mg per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy-applies. Oral administration is preferred.

The compounds of the formula I according to claim 1 and/or their physiologically acceptable salts are also used in pathological processes which are maintained or propagated by angiogenesis, in particular in tumors, restenoses, diabetic retinopathy, macular degenerative disease or rheumatois arthritis.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

For use in the subject methods, the subject compounds may be formulated with pharmaceutically active agents other than the compounds according to the invention, particularly other anti-metastatic, antitumor or anti-angiogenic agents. Angiostatic compounds of interest include angiostatin, enclostatin, carboxy terminal peptides of collagen alpha (XV), etc.

Cytotoxic and cytostatic agents of interest include adriamycin, aleran, Ara-C, BICNU, busulfan, CNNU, cisplatinum, cytoxan, daunorubicin, DTIC, 5-FU, hydrea, ifosfamicle, methotrexate, mithramycin, mitomycin, mitoxantrone, nitrogen mustard, velban, vincristine, vinblastine, VP-16, carboplatinum, fludarabine, gemcitabine, idarubicin, irinotecan, leustatin, navelbine, taxol, taxotere, topotecan, etc.

The compounds of the invention have been shown to have antiproliferative effect in an in vivo xenograft tumor model. The subject compounds are administered to a subject having a hyperproliferative disorders, e.g., to inhibit tumor growth, to decrease inflammation associated with a lymphoproliferative disorder, to inhibit graft rejection, or neurological damage due to tissue repair, etc. The present compounds are useful for prophylactic or therapeutic purposes. As used herein, the term “treating” is used to refer to both prevention of disease, and treatment of pre-existing conditions. The prevention of proliferation is accomplished by administration of the subject compounds prior to development of overt disease, e.g., to prevent the regrowth of tumors, prevent metastatic growth, diminish restenosis associated with cardiovascular surgery, etc. Alternatively the compounds are used to treat ongoing disease, by stabilizing or improving the clinical symptoms of the patient.

The host, or patient, may be from any mammalian species, e.g., primate sp., particularly human; rodents, including mice, rats and hamsters; rabbits; equines, bovines, canines, felines; etc. Animal models are of interest for experimental investigations, providing a model for treatment of human disease.

The susceptibility of a particular cell to treatment with the subject compounds may be determined by in vitro testing. Typically a culture of the cell is combined with a subject compound at varying concentrations for a period of time sufficient to allow the active agents to induce cell death or inhibit migration, usually between about one hour and one week. For in vitro testing, cultured cells from a biopsy sample may be used. The viable cells left after treatment are then counted.

The dose will vary depending on the specific compound utilized, specific disorder, patient status, etc. Typically a therapeutic dose will be sufficient to substantially decrease the undesirable cell population in the targeted tissue, while maintaining patient viability. Treatment will generally be continued until there is a substantial reduction, e.g., at least about 50%, decrease in the cell burden, and may be continued until there are essentially none of the undesirable cells detected in the body.

The compounds according to the invention are preferably administered to human or nonhuman animals, more preferred to mammalian animals and especially to humans.

The compounds also find use in the specific inhibition of a signaling pathway mediated by protein kinases. Protein kinases are involved in signaling pathways for such important cellular activities as responses to extracellular signals and cell cycle checkpoints. Inhibition of specific protein kinases provided a means of intervening in these signaling pathways, for example to block the effect of an extracellular signal, to release a cell from cell cycle checkpoint, etc. Defects in the activity of protein kinases are associated with a variety of pathological or clinical conditions, where there is a defect in the signaling mediated by protein kinases. Such conditions include those associated with defects in cell cycle regulation or in response to extracellular signals, e.g., immunological disorders, autoimmune and immunodeficiency diseases; hyperproliferative disorders, which may include psoriasis, arthritis, inflammation, endometriosis, scarring, cancer, etc. The compounds of the present invention are active in inhibiting purified kinase proteins preferably raf kinases, e.g., there is a decrease in the phosphorylation of a specific substrate in the presence of the compound. The compounds of the invention may also be useful as reagents for studying signal transduction or any of the clinical disorders listed throughout this application.

There are many disorders associated with a dysregulation of cellular proliferation. The conditions of interest include, but are not limited to, the following conditions. The subject compounds are useful in the treatment of a variety of conditions where there is proliferation and/or migration of smooth muscle cells, and/or inflammatory cells into the intimal layer of a vessel, resulting in restricted blood flow through that vessel, e.g., neointimal occlusive lesions. Occlusive vascular conditions of interest include atherosclerosis, graft coronary vascular disease after transplantation, vein graft stenosis, peri-anastomatic prothetic graft stenosis, restenosis after angioplasty or stent placement, and the like. Diseases where there is hyperproliferation and tissue remodelling or repair or reproductive tissue, e.g., uterine, testicular and ovarian carcinomas, endometriosis, squamous and glandular epithelial carcinomas of the cervix, etc. are reduced in cell number by administration of the subject compounds. The growth and proliferation of neural cells is also of interest.

Tumor cells are characterized by uncontrolled growth, invasion to surrounding tissues, and metastatic spread to distant sites. Growth and expansion requires an ability not only to proliferate, but also to down-modulate cell death (apoptosis) and activate angiogenesis to product a tumor neovasculature.

Tumors of interest for treatment include carcinomas, e.g., colon, duodenal, prostate, breast, melanoma, ductal, hepatic, pancreatic, renal, endometrial, stomach, dysplastic oral mucosa, polyposis, invasive oral cancer, non-small cell lung carcinoma, transitional and squamous cell urinary carcinoma etc.; neurological malignancies; e.g. neuroplastoma, gliomas, etc.; hematological malignancies, e.g., childhood acute leukaemia, non-Hodgkin's lymphomas, chronic lymphocytic leukaemia, malignant cutaneous T-cells, mycosis fungoides, non-MF cutaneous T-cell-lymphoma, lymphomatoid papulosis, T-cell rich cutaneous lymphoid hyperplasia, bullous pemphigoid, discoid lupus erythematosus, lichen planus, etc.; and the like.

Tumors of neural tissue are of particular interest, e.g., gliomas, neuromas, etc. Some cancers of particular interest include breast cancers, which are primarily adenocarcinoma subtypes. Ductal carcinoma in situ is the most common type of noninvasive breast cancer. In DCIS, the malignant cells have not metastasized through the walls of the ducts into the fatty tissue of the breast. Infiltration (or invasive) ductal carcinoma (IDC) has metastasized through the wall of the duct and invaded the fatty tissue of the breast. Infiltrating (or invasive) lobular carcinoma (ILC) is similar to IDC, in that it has the potential to metastasize elsewhere in the body. About 10% to 15% of invasive breast cancers are invasive lobular carcinomas.

Also of interest is non-small cell lung carcinoma. Non-small cell lung cancer (NSCLC) is made up of three general subtypes of lung cancer. Epidermoid carcinoma (also called squamous cell carcinoma) usually starts in one of the larger bronchial tubes and grows relatively slowly. The size of these tumors can range from very small to quite large. Adenocarcinoma starts growing near the outside surface of the lung and may vary in both size and growth rate. Some slowly growing adenocarcinomas are described as alveolar cell cancer. Large cell carcinoma starts near the surface of the lung, grows rapidly, and the growth is usually fairly large when diagnosed. Other less common forms of lung cancer are carcinoid, cylindroma, mucoepidermoid, and malignant mesothelioma.

Melanoma is a malignant tumor of melanocytes. Although most melanomas arise in the skin, they also may arise from mucosal surfaces or at other sites to which neural crest cells migrate. Melanoma occurs predominantly in adults, and more than half of the cases arise in apparently normal areas of the skin. Prognosis is affected by clinical and histological factors and by anatomic location of the lesion. Thickness and/or level of invasion of the melanoma, mitotic index, tumor infiltrating lymphocytes, and ulceration or bleeding at the primary site affect the prognosis. Clinical staging is based on whether the tumor has spread to regional lymph nodes or distant sites. For disease clinically confined to the primary site, the greater the thickness and depth of local invasion of the melanoma, the higher the chance of lymph node metastases and the worse the prognosis. Melanoma can spread by local extension (through lymphatics) and/or by hematogenous routes to distant sites. Any organ may be involved by metastases, but lungs and liver are common sites.

Other hyperproliferative diseases of interest relate to epidermal hyperproliferation, tissue, remodeling and repair. For example, the chronic skin inflammation of psoriasis is associated with hyperplastic epidermal keratinocycles as well as infiltrating mononuclear cells, including CD4+ memory T cells, neutrophils and macrophages.

The proliferation of immune cells is associated with a number of autoimmune and lymphoproliferative disorders. Diseases of interest include multiple sclerosis, rheumatoid arthritis and insulin dependent diabetes mellitus. Evidence suggests that abnormalities in apoptosis play a part in the pathogenesis of systemic lupus erythematosus (SLE). Other lymphoproliferative conditions the inherited disorder of lymphocyte apoptosis, which is an autoimmune lymphoproliferative syndrome, as well as a number of leukemia's and lymphomas. Symptoms of allergies to environmental and food agents, as well as inflammatory bowel disease, may also be alleviated by the compounds of the invention.

Surprisingly, it has been found that diacylhydrazine derivatives according to the invention are able to interact with signaling pathways, especially the signaling pathways described herein and preferably the raf-kinase signaling pathway. Diacylhydrazine derivatives according to the invention preferably show advantageous biological activity which can easily be demonstrated according to methods known in the art, for example by enzyme based assays. Suitable assays are known in the art, for example from the literature cited herein and the references cited in the literature, or can be developed and/or performed in an analogous manner thereof. In such enzyme based assays, diacylhydrazine derivatives according to the invention show an effect, preferably a modulating and especially an inhibiting effect which is usually documented by IC₅₀ values in a suitable range, preferably in the micromolar range and more preferred in the nanomolar range.

In general, compounds according to the invention are to be regarded as suitable kinase-modulators and especially suitable kinase-inhibitors according to the invention if they show an effect or an activity to one or more kinases, preferably to one or more raf-kinases that preferably lies, determined as IC₅₀-value, in the range of 100 μmol or below, preferably 10 μmol or below, more preferably in the range of 3 μmol or below, even more preferably in the range of 1 μmol or below and most preferably in the nanomolar range. Especially preferred for use according to the invention are kinase-inhibitors as defined above/below, that show an activity, determined as IC₅₀-value, to one or more raf-kinases, preferably including A-raf, B-raf and c-raf1 or consisting of A-raf, B-raf and c-raf1 and more preferred including c-raf1 or consisting of c-raf1, in the range of 0.5 μmol or below and especially in the range of 0.1 μmol or below. In many cases an IC₅₀-value at the lower end of the given ranges is advantageous and in some cases it is highly desirable that the IC₅₀-value is as small as possible or the he IC₅₀-values are as small as possible, but in general IC₅₀-values that lie between the above given upper limits and a lower limit in the range of 0.0001 μmol, 0.001 μmol, 0.01 μmol or even above 0.1 μmol are sufficient to indicate the desired pharmaceutical activity. However, the activities measured can vary depending on the respective testing system or assay chosen.

Alternatively, the advantageous biological activity of the compounds according to the invention can easily be demonstrated in in vitro assays, such as in vitro proliferation assays or in vitro growth assays. Suitable in vitro assays are known in the art, for example from the literature cited herein and the references cited in the literature or can be performed as described below, or can be developed and/or performed in an analogous manner thereof.

As an example for an in vitro growth assay, human tumor cell lines, for example HCT116, DLD-1 or MiaPaCa, containing mutated K-ras genes can be used in standard proliferation assays, for example for anchorage dependent growth on plastic or anchorage independent growth in soft agar. Human tumor cell lines are commercially available, for example from ATCC (Rockville Md.), and can be cultured according to methods known in the art, for example in RPMI with 10% heat inactivated fetal bovine serum and 200 mM glutamine. Cell culture media, fetal bovine serum and additives are commercially available, for example from Invitrogen/Gibco/BRL (Karlsruhe, Germany) and/or QRH Biosciences (Lenexa, Kans.). In a standard proliferation assay for anchorage dependent growth, 3×10³ cells can be seeded into 96-well tissue culture plates and allowed to attach, for example overnight at 37° C. in a 5% CO₂ incubator. Compounds can be titrated in media in dilution series and added to 96 well cell cultures. Cells are allowed to grow, for example for 1 to 5 days, typically with a feeding of fresh compound containing media at about half of the time of the growing period, for example on day 3, if the cells are allowed to grow 5 days. Proliferation can be monitored by methods known in the art, such as measuring metabolic activity, for example with standard XTT calorimetric assay (Boehringer Mannheim) measured by standard ELISA plate reader at OD 490/560, by measuring ³H-thymidine incorporation into DNA following an 8 h culture with 1 μCu ³H-thymidine, harvesting the cells onto glass fiber mats using a cell harvester and measuring ³H-thymidine incorporation by liquid scintillation counting, or by staining techniques, such as crystal violet staining. Other suitable cellular assay systems are known in the art.

Alternatively, for anchorage independent cell growth, cells can be plated at 1×10³ to 3×10³ in 0.4% Seaplaque agarose in RPMI complete media, overlaying a bottom layer containing only 0.64% agar in RPMI complete media, for example in 24-well tissue culture plates. Complete media plus dilution series of compounds can be added to wells and incubated, for example at 37° C. in a 5% CO₂ incubator for a sufficient time, for example 10-14 days, preferably with repeated feedings of fresh media containing compound, typically at 34 day intervals. Colony formation and total cell mass can be monitored, average colony size and number of colonies can be quantitated according to methods known in the art, for example using image capture technology and image analysis software. Image capture technology and image analysis software, such as Image Pro Plus or media Cybernetics.

As discussed herein, these signaling pathways are relevant for various disorders. Accordingly, by interacting with one or more of said signaling pathways, diacylhydrazine derivatives are useful in the prevention and/or the treatment of disorders that are dependent from said signaling pathways.

The compounds according to the invention are preferably kinase modulators and more preferably kinase inhibitors. According to the invention, kinases include, but are not limited to one or more Raf-kinases, one or more Tie-kinases, one or more VEGFR-kinases, one or more PDGFR-kinases, p38-kinase and/or SAPK2alpha.

Raf-kinases in this respect are respect preferably include or consist of A-Raf, B-Raf and c-Raf1.

Tie-kinases in this respect preferably include or consist of Tie-2 kinase.

VEGFR-kinases in this respect preferably include or consist of VEGFR-2 kinase.

Due to the kinase modulating or inhibiting properties of the compounds according to the invention, the compounds according to the invention preferably interact with one or more signalling pathways which are preferably cell signalling pathways, preferably by downregulating or inhibiting said signaling pathways. Examples for such signalling pathways include, but are not limited to the raf-kinase pathway, the Tie-kinase pathway, the VEGFR-kinase pathway, the PDGFR-kinase pathway, the p38-kinase pathway, the SAPK2alpha pathway and/or the Ras-pathway.

Modulation of the raf-kinase pathway plays an important role in various cancerous and noncancerous disorders, preferably cancerous disorders, such as dermatological tumors, haematological tumors, sarcomas, squamous cell cancer, gastric cancer, head cancer, neck cancer, oesophageal cancer, lymphoma, ovary cancer, uterine cancer and/or prostate cancer. Modulation of the raf-kinase pathway plays a even more important role in various cancer types which show a constitutive activation of the raf-kinase dependent signalling pathway, such as melanoma, colorectal cancer, lung cancer, brain cancer, pancreatic cancer, breast cancer, gynecological cancer, ovarian cancer, thyroid cancer, chronic leukaemia and acute leukaemia, bladder cancer, hepatic cancer and/or renal cancer. Modulation of the raf-kinase pathway plays also an important role in infection diseases, preferably the infection diseases as mentioned above/below and especially in Helicobacter pylori infections, such as Helicobacter pylori infection during peptic ulcer disease.

One or more of the signalling pathways mentioned above/below and especially the VEGFR-kinase pathway plays an important role in angiogenesis. Accordingly, due to the kinase modulating or inhibiting properties of the compounds according to the invention, the compounds according to the invention are suitable for the prophylaxis and/or treatment of pathological processes or disorders caused, mediated and/or propagated by angiogenesis, for example by inducing anti-angiogenesis. Pathological processes or disorders caused, mediated and/or propagated by angiogenesis include, but are not limited to tumors, especially solid tumors, arthritis, especially heumatic or rheumatoid arthritis, diabetic retinopathy, psoriasis, restenosis; fibrotic disorders; mesangial cell proliferative disorders, diabetic nephropathy, malignant nephrosclerosis, thrombotic microangiopathy syndromes, organ transplant rejection, glomerulopathies, metabolic disorders, inflammation and neurodegenerative diseases, and especially solid tumors, rheumatic arthritis, diabetic retinopathy and psoriasis.

Modulation of the p38-signalling pathway plays an important role in various cancerous and although in various noncancerous disorders, such as fibrosis, atherosclerosis, restenosis, vascular disease, cardiovascular disease, inflammation, renal disease and/or angiogenesis, and especially noncancerous disorders such as rheumatoid arthritis, inflammation, autoimmune disease, chronic obstructive pulmonary disease, asthma and/or inflammatory bowel disease.

Modulation of the PDGF-signalling pathway plays an important role in various cancerous and although in various noncancerous disorders, such as rheumatoid arthritis, inflammation, autoimmune disease, chronic obstructive pulmonary disease, asthma and/or inflammatory bowel disease, and especially noncancerous disorders such as fibrosis, atherosclerosis, restenosis, vascular disease, cardiovascular disease, inflammation, renal disease and/or angiogenesis.

Subject of the present invention are therefore diacylhydrazine derivatives according to the invention as promoters or inhibitors, preferably as inhibitors, of the signaling pathways described herein. Preferred subject of the invention are therefore diacylhydrazine derivatives according to the invention as promoters or inhibitors, preferably as inhibitors of the raf-kinase pathway. More preferred subject of the invention are therefore diacylhydrazine derivatives according to the invention as promoters or inhibitors, preferably as inhibitors of the raf-kinase. Even more preferred subject of the invention are diacylhydrazine derivatives according to invention as promoters or inhibitors, preferably as inhibitors of one or more raf-kinases, selected from the group consisting of A-raf, B-raf and c-raf1. Especially preferred subject of the invention are diacylhydrazine derivatives according to the invention as promoters or inhibitors, preferably as inhibitors of c-raf1.

Thus, subject of the present invention are diacylhydrazine derivatives according to the invention as medicaments. Subject of the present invention are diacylhydrazine derivatives according to the invention as medicament active ingredients. Further subject of the present invention is the use of one or more diacylhydrazine derivatives according to the invention as a pharmaceutical. Further subject of the present invention is the use of one or more diacylhydrazine derivatives according to the invention in the treatment and/or the prophylaxis of disorders, preferably the disorders described herein, more preferred disorders that are caused, mediated and/or propagated by signalling pathways discussed herein, even more preferred disorders that are caused, mediated and/or propagated by raf-kinases and especially disorders that are caused, mediated and/or propagated by raf-kinases, selected from the group consisting of A-raf, B-raf and c-raf1. Usually, the disorders discussed herein are divided into two groups, hyperproliferative and non hyperproliferative disorders. In this context, psioarsis, arthritis, inflammation, endometriosis, scarring, begnin prostatic hyperplasia, immunological diseases, autoimmune diseases and immunodeficiency diseases are to be regarded as noncancerous disorders, of which arthritis, inflammation, immunological diseases, autoimmune diseases and immunodeficiency diseases are usually regarded as non hyperproliferative-disorders. In this context, brain cancer, lung cancer, squamous cell cancer, bladder cancer, gastric cancer, pancreatic cancer, hepatic cancer, renal cancer, colorectal cancer, breast cancer, head cancer, neck cancer, oesophageal cancer, gynecological cancer, thyroid cancer, lymphoma, chronic leukaemia and acute leukaemia are to be regarded as cancerous disorders, all of which are usually regarded as hyperproliferative disorders. Especially cancerous cell growth and especially cancerous cell growth mediated by raf-kinase is a disorder which is a target of the present invention. Subject of the present invention therefore are diacylhydrazine derivatives according to the invention as medicaments and/or medicament active ingredients in the treatment and/or the prophylaxis of said disorders and the use of diacylhydrazine derivatives according to the invention for the manufacture of a pharmaceutical for the treatment and/or the prophylaxis of said disorders as well as a method of treatment of said disorders, comprising administering one or more diacylhydrazine derivatives according to the invention to a patient in need of such an administration. Subject of the present invention therefore are diacylhydrazine derivatives according to the invention as medicaments and/or medicament active ingredients in the treatment and/or the prophylaxis said disorders and the use of diacylhydrazine derivatives according to the invention for the manufacture of a pharmaceutical for the treatment and/or the prophylaxis of said disorders as well as a method of treatment of said disorders, comprising administering one or more diacylhydrazine derivatives according to the invention to a patient in need of such an administration.

Accordingly, subject of the present invention are pharmaceutical compositions that contain one or more diacylhydrazine derivatives according to the invention. Subject of the present invention are especially pharmaceutical compositions that contain one or more diacylhydrazine derivatives according to the invention and one or more additional compounds (other than the compounds of the instant invention), preferably selected from the group consisting of physiologically acceptable excipients, auxiliaries, adjuvants, carriers and pharmaceutically active ingredients other than the compounds according to the invention.

Accordingly, subject of the present invention is a process for the manufacture of a pharmaceutical composition, wherein one or more diacylhydrazine derivatives according to the invention and one or more compounds (other than the compounds of the instant invention), preferably selected from the group consisting of carriers, excipients, auxiliaries, adjuvants and pharmaceutically active ingredients other than the compounds according to the invention.

Accordingly, the use of the compounds according to the invention in the treatment of Hyperproliferative disorders is a subject of the instant invention.

Accordingly, the use of the compounds according to the invention for producing a medicament for the treatment of hyperproliferative disorders is a subject of the instant invention.

The present invention relates to diacylhydrazine derivatives of formula I, the use of the compounds of formula I as inhibitors of raf-kinase, the use of the compounds of formula I for the manufacture of a pharmaceutical composition and a method of treatment, comprising administering said pharmaceutical composition to a patient.

Examples for Chemical Syntheses

Above and below, all temperatures are given in ° C. In the examples below, “conventional work-up” means that the organic phase is washed with saturated NaHCO₃ solution, if desired with water and saturated NaCl solution, the phases are separated, the organic phase is dried over sodium sulfate and evaporated, and the product is purified by chromatography on silica gel, by preparative HPLC and/or by crystallization.

### 1) Synthesis of (4-Chloropyridine-2-carboxylic acid)-methylamide (2)

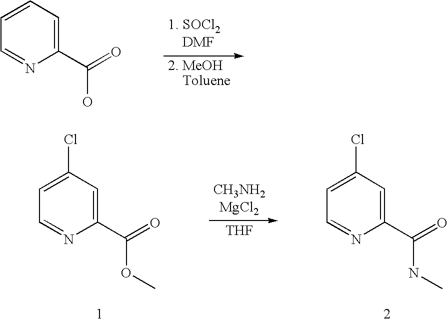

60 ml of thionylchloride are heated to 45° C. under a N₂ atmosphere, and slowly mixed with 1.83 ml dimethyl formamide. To this solution, 20 g of pyridine-2-carboxylic acid are added portion wise. The reaction mixture is stirred for another 15 min at 45° C. and subsequently kept at 80° C. for 24 hours. The reaction mixture is evaporated to dryness, the residue is stripped several times with water free toluene. The oil obtained by this procedure is dissolved in toluene, chilled to 0° C., slowly mixed with methanol and stirred for 1 hour. The precipitated solid is separated by suction filtration, washed with toluene and recrystallized from acetone. Yield: 15 g (44%) of 1, colorless crystals

13 g (62.5 mmol) of 1 are dissolved in THF together with 2.98 g (31.2 mmol) of water free magnesium chloride. After 5 min, 110 ml methylamine solution (2M in THF) are added dropwise within 10 min. This suspension is stirred for 2 hours at room temperature. The reaction mixture is mixed with 120 ml water and 63 ml 1 N HCl solution and extracted 3× with ethyl acetate. The pooled organic phases are washed with saturated NaCl solution, dried with Na₂SO₄, filtered and evaporated to dryness.

Yield: 10.5 g (98.5%) of 2, colorless oil

### 2) Synthesis of 3-(2-Methylcarbamoyl-pyridine-4-yloxy)-benzoic acid (4)

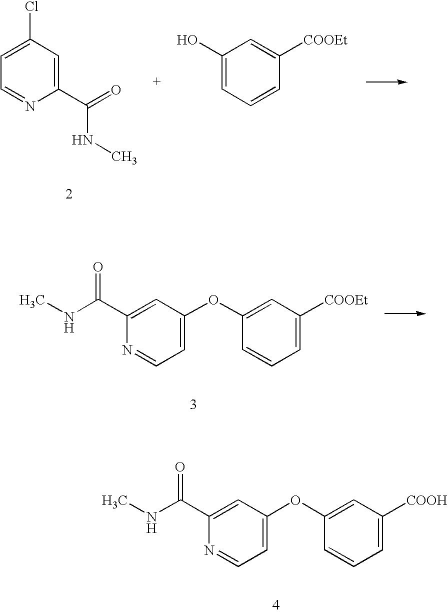

A molten mixture of 5.0 g (29.3 mmol) of 2 and 9.74 g (58.6 mmol) of 3-Hydroxybenzoic acid ethyl ester is stirred at 160° C. for 15 hours. The reaction mixture is chilled down, mixed with acetic acid ethyl ester and extracted twice each with 1 N caustic soda solution and water. The organic phase is dried over sodium sulfate. Reaction product 3 is obtained upon filtering and distilling off the solvent.

Yield: 5.80 g (56.5%) 3, brownish oil.

5.80 g (16.6 mmol) of 3 are dissolved in 100 ml ethanol and mixed with 200 ml of 1 N caustic soda solution. After stirring for 1 hour at room temperature, the mixture is concentrated and extracted with acetic acid ethyl ester. The pH value of the aqueous phase is adjusted to pH 4 with hydrochloric acid. The precipitated solid is filtered off.

Yield: 2.85 g (63.2%) 4, white solid.

### 3) Synthesis of 3-(2-Methylcarbamoyl-pyridine-4-yloxy)-benzoic acid pentafluoro-phenyl ester (5)

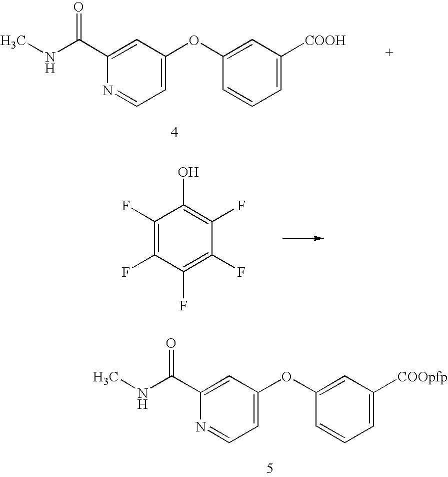

2.80 g (10.3 mmol) of 4 are dissolved in 50 ml 1,4 dioxane in an inert gas atmosphere and mixed with 1.9 g (10.3 mmol) of pentafluorophenol and 2.13 g (10.3 mmol) of N,N-dicyclohexyl carbodiimide. After stirring for 15 hours at room temperature, the precipitated solid is filtered off and the solvent distilled off. The obtained crude product is purified by means of normal phase column chromatography (eluent: petrol ether/acetic acid ethyl ester).

Yield: 3.60 g (79.2%) 5, yellowish solid.

### 4) Synthesis of 4-{3-[N′-(3-Bromo-benzoyl)-hydrazinocarbonyl]-phenoxy}-pyridine-2-carboxylic acid methyl amide (6)

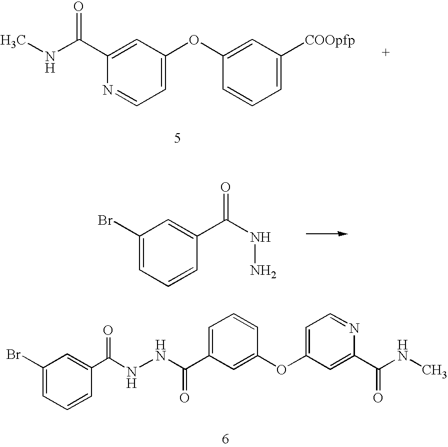

100 mg (0.23 mmol) of 5 are dissolved in 2.0 ml N,N-dimethyl formamide and mixed with 50 mg (0.23 mmol) of 3-bromobenzhydrazide. The mixture is stirred for 72 hours at 55° C. After removing the solvent by means of a vacuum centrifuge, the residue is dissolved in dichloromethane and crystallized from dichloromethane/tert.-butyl-methyl ether.

Yield: 77.0 mg (70.7%) 6, beige crystals.

Analogous arylhydrazides are obtainable according to standard procedures from the corresponding methyl esters by reaction with hydrazinium hydroxide.

### 5) Synthesis of 4-pyridine-4-yl-benzoic acid, sodium salt (7)

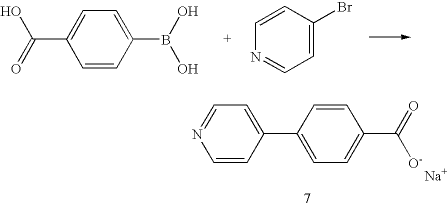

5.0 g (29.3 mmol) of 4-carboxybenzeneboronic acid are dissolved in 150 ml of acetonitrile and mixed with 5.7 g (29.3 mmol) of 4-bromo-pyridinium chloride, 1.72 g (1.5 mmol) of tetrakis(triphenylphosphine)palladium (0) and 150 ml 0.5 M of sodium carbonate solution in an inert gas atmosphere.

The reaction mixture is refluxed for 15 hours. After stopping the reaction the solvent is distilled off, the residue is suspended in water and the pH value is adjusted to 7 with hydrochloric acid. Finally, the precipitated crystals are filtered off.

Yield: 4.78 g (88.8%) 7, white powder.

The further synthesis using derivative 7 is carried out as described above, analogously to the synthesis examples 3) and 4).

### EXAMPLES FOR DELIVERY SYSTEMS

### Example A

### Injection Vials

A solution of 100 g of an active compound of the formula I and 5 g of disodium hydrogenphosphate is adjusted to pH 6.5 in 3 l of double-distilled water using 2N hydrochloric acid, sterile-filtered, dispensed into injection vials, lyophilized under sterile conditions and aseptically sealed. Each injection vial contains 5 mg of active compound.

### Example B

### Suppositories

A mixture of 20 g of an active compound of the formula I is fused with 1100 g of soya lecithin and 1400 g of cocoa butter, poured into moulds and allowed to cool. Each suppository contains 20 mg of active compound.

### Example C

### Solution

A solution of 1 g of an active compound of the formula I, 9.38 g of NaH₂PO₄.2H₂O, 28.48 g of Na₂HPO₄.12H₂O and 0.1 g of benzalkonium chloride in 940 ml of double-distilled water is prepared. It is adjusted to pH 6.8, made up to 1 l and sterilized by irradiation. This solution can be used in the form of eye drops.

### Example D

### Ointment

500 mg of an active compound of the formula I is mixed with 99.5 g of petroleum jelly under aseptic conditions.

### Example E

### Tablets

A mixture of 1 kg of active compound of the formula I, 4 kg of lactose, 1.2 kg of potato starch, 0.2 kg of talc and 0.1 kg of magnesium stearate is compressed to give tablets in a customary manner such that each tablet contains 10 mg of active compound.

### Example F

### Coated Tablets

Analogously to Example E, tablets are pressed and are then coated in a customary manner using a coating of sucrose, potato starch, talc, tragacanth and colourant.

### Example G

### Capsules

2 kg of active compound of the formula I are dispensed into hard gelatin capsules in a customary manner such that each capsule contains 20 mg of the active compound.

### Example H

### Ampoules

A solution of 1 kg of active compound of the formula I in 60 l of double-distilled water is sterile-filtered, dispensed into ampoules, lyophilized under sterile conditions and aseptically sealed. Each ampoule contains 10 mg of active compound.

1. Diacylhydrazine derivatives of formula I
A-D-B (I)
wherein
D is a bivalent diacylhydrazine moiety, or a derivative thereof,
A is a unsubstituted or substituted moiety of up to 40 carbon atoms of the formula: -L-(M-L′)_{α}, where L is a 5, 6 or 7 membered cyclic structure, selected from the group consisting of aryl, heteroaryl, arylene and heteroarylene, bound directly to D, L′ comprises an optionally substituted cyclic moiety having at least 5 members, selected from the group consisting of aryl, heteroaryl, aralkyl, cycloalkyl and heterocyclyl, M is a bond or a bridging group having at least to one atom, α is an integer of from 1-4; and each cyclic structure of L and L′ contains 0-4 members of the group consisting of nitrogen, oxygen and sulfur, wherein L′ is substituted by at least one substituent selected from the group consisting of —SO_{β}Rₓ, —C(O)Rₓ and —C(NR_{y})R_{z},
B is a substituted or unsubstituted, up to tricyclic aryl or heteroaryl moiety of up to 30 carbon atoms, comprising at least one 5-, 6-, or 7-membered cyclic structure, bound directly to D containing 0-4 members of the group consisting of nitrogen, oxygen and sulfur, wherein said cyclic structure directly bound to D is selected from the group consisting of aryl, heteroaryl and heterocyclyl,
R_{y} is hydrogen or a carbon based moiety of up to 24 carbon atoms optionally containing heteroatoms selected from N, S and O and optionally halosubstituted, up to per halo,
R_{z} is hydrogen or a carbon based moiety of up to 30 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen;
Rₓ is R_{z} or NRₐR_{b}, where Rₐ and R_{b} are
a) independently hydrogen, a carbon based moiety of up to 30 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen, or
—OSi(R_{f})₃ where R_{f} is hydrogen or a carbon based moiety of up to 24 carbon atoms optionally containing heteroatoms selected from N, S and O and optionally substituted by halogen, hydroxy and carbon based substituents of up to 24 carbon atoms, which optionally contain heteroatoms selected from N, S and O and are optionally substituted by halogen;
or
b) Rₐ and R_{b} together form a 5-7 member heterocyclic structure of 1-3 heteroatoms selected from N, S and O, or a substituted 5-7 member heterocyclic structure of 1-3 heteroatoms selected from N, S and O substituted by halogen, hydroxy or carbon based substituents of up to 24 carbon atoms, which optionally contain heteroatoms selected from N, S and O and are optionally substituted by halogen; or
c) one of Rₐ or R_{b} is —C(O)—, a C₁-C₅ divalent alkylene group or a substituted C₁-C₅ divalent alkylene group bound to the moiety L to form a cyclic structure with at least 5 members, wherein the substituents of the substituted C₁-C₅ divalent alkylene group are selected from the group consisting of halogen, hydroxy, and carbon based substituents of up to 24 carbon atoms, which optionally contain heteroatoms selected from N, S and O and are optionally substituted by halogen;
where B is substituted, L is substituted or L′ is additionally substituted, the substituents are selected from the group consisting of halogen, up to per-halo, and Wγ, where γ is 0-3;
wherein each W is independently selected from the group consisting of —CN, —CO₂R, —C(O)NR⁵R⁵, —C(O)—R⁵, —NO₂, —OR⁵, —SR⁵, —NR⁵R⁵, —NR⁵C(O)OR⁵, —NR⁵C(O)R⁵, -Q-Ar, and carbon based moieties of up to 24 carbon atoms, optionally containing heteroatoms selected from N, S and O and optionally substituted by one or more substituents independently selected from the groups consisting of —CN, —CO₂R, —C(O)NR⁵R⁵, —C(O)—R⁵, —NO₂, —OR⁵, —SR⁵, —NR⁵R⁵, —NR⁵C(O)OR⁵, —NR⁵C(O)R⁵ and halogen up to per-halo; with each R⁵ independently selected from H or a carbon based moiety of up to 24 carbon atoms, optionally containing heteroatoms selected from N, S and O and optionally substituted by halogen, wherein Q is —O—, —S—, —N(R⁵)—, —(CH₂)O, —C(O)—, —CH(OH)—, —(CH₂)_{β}—, —(CH₂)_{β}S—, —(CH₂)_{β}N(R⁵)—, —O(CH₂)_{β}—CHHal-, —CHal₂-, —S—(CH₂)— and —N(R⁵)(CH₂)_{β}— where β=1-3, and Hal is halogen; and
Ar is 5- or 6-member aromatic structure containing 0-2 members selected from the group consisting of nitrogen, oxygen and sulfur, which is optionally substituted by halogen, up to per-halo, and optionally substituted by Zδ1 wherein δ1 is 0 to 3 and each Z is independently selected from the group consisting —CN, —CO₂R⁵, —C(O)NR⁵R⁵, —C(O)—R⁵, —NO₂, —OR⁵, —SR⁵, —SO₂R⁵, —SO₃H, —NR⁵R⁵, —NR⁵C(O)OR⁵, —NR⁵C(O)R⁵, and a carbon based moiety of up to 24 carbon atoms, optionally containing heteroatoms selected from N, S and O and optionally substituted by one or more substituents selected from the group consisting of —CN, —CO₂R⁵, —C(O)NR⁵R⁵, —C(O)—R⁵, —NO₂, —OR⁵, —SR⁵, —SO₂R⁵, —SO₃H, —NR⁵R⁵, —NR⁵C(O)OR⁵, —NR⁵C(O)R⁵, and the pharmaceutically acceptable derivatives, salts and solvates thereof.

2. Diacylhydrazine derivative according to claim 1, characterised in that each M independently from one another represents a bond or is a bridging group, selected from the group consisting of (CR⁵R⁵)ₕ, or (CHR⁵)ₕ-Q-(CHR⁵)ᵢ, wherein
Q is selected from a group consisting of O, S, N—R⁵, CR¹⁵H¹⁶, (CHal₂)ⱼ, (O—CHR⁵)ⱼ, (CHR⁵—O)ⱼ, CR⁵═CR⁵, (O—CHR⁵CHR⁵)ⱼ, (CHR⁵CHR⁵—O)ⱼ, C═O, C═S, C═NR⁵, CH(OR⁵), C(OR⁵)(OR⁵), C(═O)O, OC(═O), OC(═O)O, C═O)N(R⁵)C(═O), OC(═O)N(R⁵), N(R⁵)C(═O)O,
CH═N—NR⁵, OC(O)NR⁵, NR⁵C(O)O, S═O, SO₂, SO₂NR⁵ und NR⁵SO₂, wherein
R⁵ is in each case independently selected from the meanings given above, preferably hydrogen, halogen, alkyl, aryl, aralkyl,
h, i are independently from each other 0, 1, 2, 3, 4, 5, or 6, and
j is 1, 2, 3, 4, 5 or 6.

3. Diacylhydrazine derivative according to claim 1, selected from the compounds of formula II, 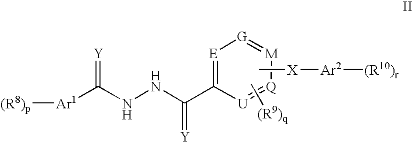
wherein
Ar¹, Ar² are selected independently from one another from aromatic hydrocarbons containing 6 to 14 carbon atoms and ethylenical unsaturated or aromatic heterocyclic residues containing 3 to 10 carbon atoms and one two or three hetero atoms, independently selected from N, O und S,
E, G, M, Q
and U are selected, independently from one another, from carbon atoms and nitrogen atoms, with the proviso that one or more of E, G, M, Q and U are carbon atoms and that X is bonded to a carbon atom,
R⁸, R⁹ and
R¹⁰ are independently selected from a group consisting of H, A, OA, cycloalkyl comprising 3 to 7 carbon atoms, Hal, CH₂Hal, CH(Hal)₂, C(Hal)₃, NO₂, (CH₂)ₙCN, (CH₂)ₖNR¹¹R¹², (CH₂)ₙO(CH₂)ₖNR¹¹R¹², (CH₂)ₙNR¹¹(CH₂)ₖNR¹¹R¹² (CH₂)ₙO(CH₂)ₖOR¹¹, (CH₂)ₙNR¹¹(CH₂)ₖOR¹², (CH₂)ₙCOOR¹³ (CH₂)ₙCOR³, (CH₂)ₙCONR¹¹R¹², (CH₂)ₙNR¹¹COR¹², (CH₂)ₙNR⁸CONR¹¹R¹², (CH₂)ₙNR¹¹SO₂A, (CH₂)ₙSO₂NR¹¹R¹², (CH₂)ₙS(O)ᵤR¹³, (CH₂)ₙOC(O)R³, (CH₂)ₙCOR¹³, (CH₂)ₙSR¹¹, CH═N—OA, CH₂CH═N—OA, (CH₂)ₙNHOA, (CH₂)ₙCH═N—R¹¹, (CH₂)ₙOC(O)NR¹¹R¹², (CH₂)ₙNR¹¹COOR¹³, (CH₂)ₙN(R¹¹)CH₂CH₂OR¹³, (CH₂)ₙN(R¹¹)CH₂CH₂OCF₃, (CH₂)ₙN(R¹¹)C(R¹³)HCOOR², (CH₂)ₙN(R¹¹)C(R¹³)HCOR¹¹, (CH₂)ₙN(R¹¹)CH₂CH₂N(R¹²)CH₂COOR¹¹, (CH₂)ₙN(R¹¹)CH₂CH₂NR¹¹R¹², CH═CHCOOR¹³, CH═CHCH₂NR¹¹R¹², CH═CHCH₂NR¹¹R¹², CH═CHCH₂OR¹³, (CH₂)ₙN(COOR¹³)COOR¹⁴, (CH₂)ₙN(CONH₂)COOR¹³, (CH₂)ₙN(CONH₂)CONH₂, (CH₂)ₙN(CH₂COOR³)COOR¹⁴, (CH₂)ₙN(CH₂CONH₂)COOR³, (CH₂)ₙN(CH₂CONH₂)CONH₂, (CH₂)ₙCHR¹³COR¹⁴, (CH₂)ₙCHR¹³COOR¹⁴, (CH₂)ₙCHR¹³CH₂OR⁴, (CH₂)ₙOCN and (CH₂)ₙNCO, wherein
R¹¹, R¹² are independently selected from a group consisting of H, A, (CH₂)ₘAr³ and (CH₂)ₘHet, or in NR¹¹R¹²,
R¹¹ and R¹² form, together with the N-atom they are bound to, a 5-, 6- or 7-membered heterocycles which optionally contains 1 or 2 additional hetero atoms, selected from N, O and S,
R¹³, R¹⁴ are independently selected from a group consisting of H, Hal, A, (CH₂)ₘAr⁴ and (CH₂)ₘHet,
A is selected from the group consisting of alkyl, alkenyl, cycloalkyl, alkylenecycloalkyl, alkoxy, alkoxyalkyl and saturated heterocyclyl,
Ar³, Ar⁴ are independently from one another aromatic hydrocarbon residues comprising 5 to 12 carbon atoms which are optionally substituted by one or more substituents, selected from a group consisting of A, Hal, NO₂, CN, OR¹⁵, NR¹⁵R¹⁶, COOR¹⁵, CONR¹⁵R¹⁶, NR¹⁵COR⁶, NR¹⁵CONR¹⁵R¹⁶, NR¹⁶SO₂A, COR¹⁵, SO₂R¹⁵R¹⁶, S(O)ᵤA and OOCR¹⁵,
Het is a saturated, unsaturated or aromatic heterocyclic residue which is optionally substituted by one or more substituents, selected from a group consisting of A, Hal, NO₂, CN, OR¹⁵, NR¹⁵R¹⁶, COOR⁵, CONR⁵R⁶, NR¹⁵COR⁶, NR¹⁵CONR¹⁵R¹⁶, NR¹⁶SO₂A, COR¹⁵, SO₂R¹⁵R¹⁶, S(O)ₙA and OOCR¹⁵,
R¹⁵, R¹⁶ are independently selected from a group consisting of H, A, and (CH₂)ₘAr⁶, wherein
Ar⁶ is a 5- or 6-membered aromatic hydrocarbon which is optionally substituted by one or more substituents selected from a group consisting of methyl, ethyl, propyl, 2-propyl, tert.-butyl, Hal, CN, OH, NH₂ and CF₃,
k, n and
m are independently of one another 0, 1, 2, 3, 4, or 5;
X represents a bond or is (CR¹¹R¹²)ₕ, or (CHR¹¹)ₕ-Q-(CHR¹²)ᵢ, wherein
Q is selected from a group consisting of T, CH¹⁵H¹⁶, (CHal₂)ⱼ, (O—CHR¹⁸)ⱼ, (CHR¹⁸—O)ⱼ, CR¹⁸═CR¹⁹, (O—CHR¹⁸CHR¹⁹)ⱼ, CHR¹⁸CHR¹⁹—O)ⱼ, C═O, C═S, C═NR¹⁵, CH(OR¹⁵), C(OR¹⁵)(OR², C(═O)O, OC(═O), OC(═O)O, C(═)N(R¹⁵), N(R¹⁵)C(═O), OC(═O)N(R¹⁵), N(R¹⁵)C(═O)O, CH═N—O, CH═N—NR¹⁵, OC(O)NR¹⁵, NR¹⁵C(O)O, S═O, SO₂, SO₂NR⁵ und NR¹⁵SO₂, wherein
T is selected from O, S. N—R¹⁵,
h, i are independently from each other 0, 1, 2, 3, 4, 5 or 6, and
j is 1, 2, 3, 4, 5 or 6,
Y is selected from O/S, NR²¹, C(R²²)—NO₂, C(R²²)—CN and C(CN)₂, wherein
O/S is selected from O, S,
R²¹ is independently selected from the meanings given for R¹³, R¹⁴, and
R²² is independently selected from the meanings given for R¹¹, R¹²,
p, r are independently from one another 0, 1, 2, 3, 4 or 5,
q is 0, 1, 2, 3 or 4,
u is 0, 1, 2 or 3,
and
Hal is independently selected from a group consisting of F, Cl, Br and I,
and the pharmaceutically acceptable derivatives, salts and solvates thereof.

4. Diacylhydrazine derivatives according to claim 1, selected from the compounds of formula IIa, IIb, IIc, IId, IIe, IIf, IIg, IIh, IIi, IIj, IIk, IIl, IIm, IIn, IIo, IIp, IIq, IIr, IIu, IIv, IIw and IIx, 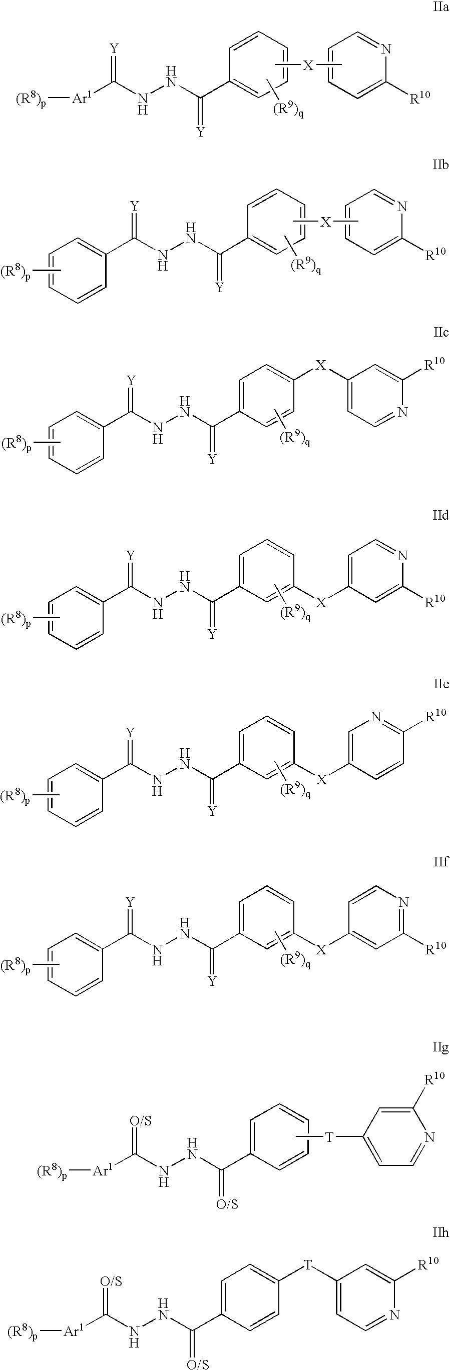 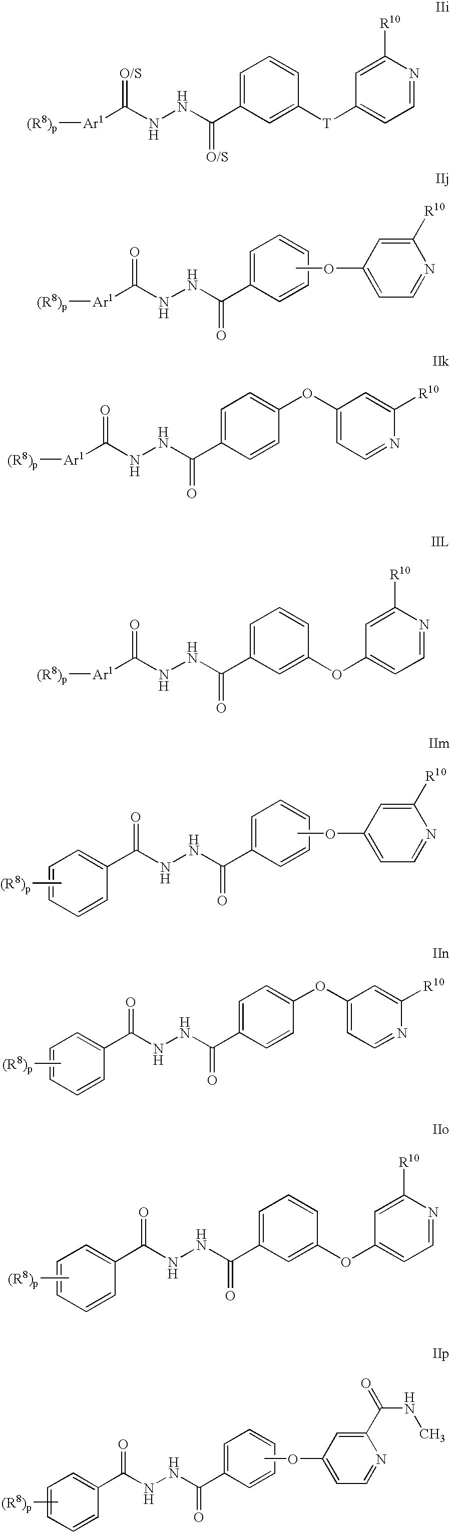 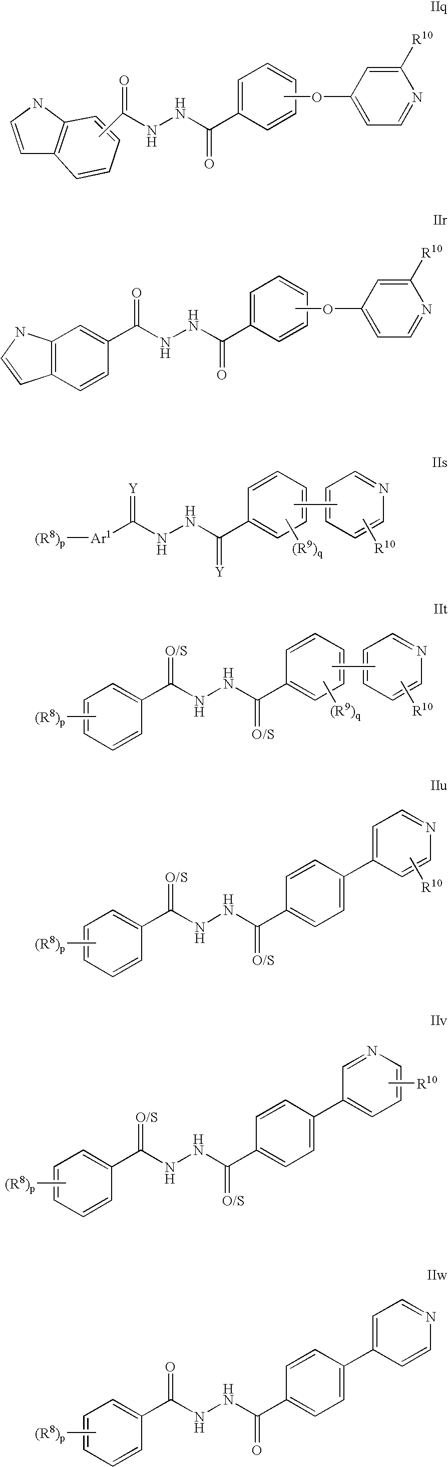 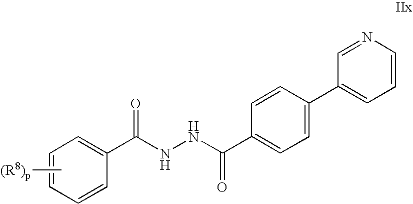
wherein R⁶, R⁷, R⁸, p, Ar¹, Y, X, R⁹ and q are as defined in claim 3, R¹⁰ is H or as defined in claim 3; and the pharmaceutically acceptable derivatives, salts and solvates thereof.

5. Diacylhydrazine derivative selected from the compounds of formula II as defined in claim 3, wherein
E, G, M, U and Q are carbon atoms,
X is O or a bond,
Y is O,
Ar¹ is phenyl or indolyl,
Ar² is pyridinyl,
R⁸ is H, methyl, ethyl, n-propyl, isopropyl, n-butyl, 2-butyl, tert.-butyl, methoxy, ethoxy, n-propoxy, isopropoxy, n-butoxy, 2-butoxy, tert.-butoxy, Hal, CHal₃ or OCHal₃,
R¹⁰ is H or CONCH₃,
p is 0, 1, 2 or 3,
q is 0 and
r is 1
and the pharmaceutically acceptable derivatives, salts and solvates thereof.

6. Diacylhydrazine derivative according to claim 5, wherein
X is O and
R¹⁰ is CONCH₃
and the pharmaceutically acceptable derivatives, salts and solvates thereof.

7. Diacylhydrazine derivative according to claim 5, wherein
X is a bond and
R¹⁰ is H
and the pharmaceutically acceptable derivatives, salts and solvates thereof.

8. Diacylhydrazine derivative selected from the compounds of formula II as defined in claim 3, wherein
E, G, M, U and Q are carbon atoms,
X is O, S or NR⁵ and
Y is O
and the pharmaceutically acceptable derivatives, salts and solvates thereof.

9. Diacylhydrazine derivative according to claim 1, selected from the compounds (1) to (224) of table I and the compounds (225) to (384) of table 2, and the pharmaceutically acceptable derivatives, salts and solvates thereof.

10. Diacylhydrazine derivative according to claim 1 as a medicament.

11. Diacylhydrazine derivative according to claim 1 as a kinase inhibitor.

12. Diacylhydrazine derivative according to claim 11, characterized in that the kinases are selected from raf-kinases.

13. Pharmaceutical composition, characterised in that it contains one or more compounds according to claim 1.

14. Pharmaceutical composition according to claim 13, characterised in that it contains one or more additional compounds, selected from the group consisting of physiologically acceptable excipients, auxiliaries, adjuvants, carriers and pharmaceutical active ingredients.

15. Process for the manufacture of a pharmaceutical composition, characterised in that one or more compounds according to claim 1 and one or more compounds, selected from the group consisting of carriers, excipients, auxiliaries and pharmaceutical active ingredients other than the compounds according to claim 1 is processed by mechanical means into a pharmaceutical composition that is suitable as dosage form for application and/or administration to a patient.

16. Use of a compound according to claim 1 as a pharmaceutical.

17. Use of a compound according to claim 1 in the treatment and/or prophylaxis of disorders.

18. Use of a compound according to claim 1 for producing a pharmaceutical composition for the treatment and/or prophylaxis of disorders.

19. Use according to claim 17, characterised in that the disorders are caused, mediated and/or propagated by raf-kinases.

20. Use according to claim 17, characterised in that the disorders are selected from the group consisting of hyperproliferative and nonhyperproliferative disorders.

21. Use according to claim 17, characterised in that the disorder is cancer.

22. Use according to claim 17, characterised in that the disorder is noncancerous.

23. Use according to claim 22, characterised in that the disorders are selected from the group consisting of psioarsis, arthritis, inflammation, endometriosis, scarring, Helicobacter pylori infection, Influenza A, begnin prostatic hyperplasia, immunological diseases, autoimmune diseases and immunodeficiency diseases.

24. Use according to claim 17, characterised in that the disorders are selected from the group consisting of melanoma, brain cancer, lung cancer, squamous cell cancer, bladder cancer, gastric cancer, pancreatic cancer, hepatic cancer, renal cancer, colorectal cancer, breast cancer, head cancer, neck cancer, oesophageal cancer, gynecological cancer, ovarian cancer, ovary cancer, uterine cancer, prostate cancer, thyroid cancer, lymphoma, chronic leukaemia and acute leukaemia.

25. Use according to claim 17, characterised in that the disorders are selected from the group consisting of arthritis, restenosis; fibrotic disorders; mesangial cell proliferative disorders, diabetic nephropathy, malignant nephrosclerosis, thrombotic microangiopathy syndromes, organ transplant rejection, glomerulopathies, metabolic disorders, inflammation, solid tumors, rheumatic arthritis, diabetic retinopathy, and neurodegenerative diseases.

26. Use according to claim 17, characterised in that the disorders are selected from the group consisting of rheumatoid arthritis, inflammation, autoimmune disease, chronic obstructive pulmonary disease, asthma, inflammatory bowel disease, fibrosis, atherosclerosis, restenosis, vascular disease, cardiovascular disease, inflammation, renal disease and angiogenesis disorders.

27. Use of a compound according to claim 1 as a raf-kinase inhibitor.

28. Use according to claim 27, characterised in that the raf-kinase is selected from the group consisting of A-Raf, B-Raf and c-Raf1.

29. Method for the treatment and/or prophylaxis of disorders, characterised in that one or more compounds according to claim 1 is administered to a patient in need of such a treatment.

30. Method according to claim 29, characterised in that the one or more compounds are administered as a pharmaceutical composition.

31. Method for the treatment and/or prophylaxis of disorders according to claim 30, characterised in that the disorders is cancerous cell growth mediated by raf kinase.

32. Method for the treatment according to claim 31, characterised in that the disorder is cancerous cell growth mediated by raf-kinase.

33. Method for producing compounds of formula II, characterised in that
a) a compound of formula III 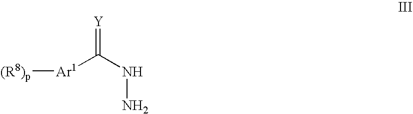
wherein Y, R⁸, p and Ar¹ are as defined in claim 3,
is reacted
b) with a compound of IV, 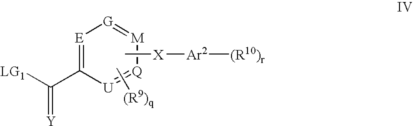
wherein
LG₁ is a leaving group, preferably a leaving group selected from OR²⁵, wherein R²⁵ is selected from the group consisting of unsubstituted or substituted aromatic residues, unsubstituted or substituted heteroaromatic residues and (O)₂S—R²⁶, wherein R²⁶ is selected from unsubstituted or substituted aromatic residues and unsubstituted or substituted alkyl residues, and wherein E, G, M, Q, U, R⁹, q, X, Ar², R¹⁰ and r are as defined in claim 3,
and optionally
c) isolating and/or treating the compound of formula II obtained by said reaction with an acid, to obtain the salt thereof.

34. Compound of formula III, 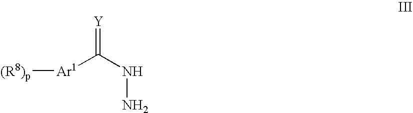
wherein Y, R⁸, p and Ar¹ are as defined in claim 3.

35. Compound of formula IV, 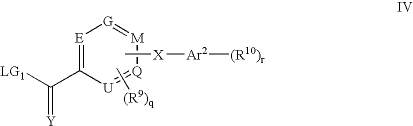
wherein
LG₁ is a leaving group, preferably a leaving group selected from OR²⁵, wherein R²⁵ is selected from the group consisting of unsubstituted or substituted aromatic residues, unsubstituted or substituted heteroaromatic residues and (O)₂S—R²⁶, wherein R²⁶ is selected from unsubstituted or substituted aromatic residues and unsubstituted or substituted alkyl residues, and wherein E, G, M, Q, U, R⁹, q, X, Ar², R¹⁰ and r are as defined in claim 3.
